(19) United States
(12) Patent Application Publication
(10) Pub. No.: US 2022/0363748 A1
(43) Pub. Date: Nov. 17, 2022

(54) ANTI IL-33 THERAPEUTIC AGENT FOR TREATING RENAL DISORDERS

(71) Applicant: MedImmune Limited, Cambridge (GB)

(72) Inventors: David James BAKER, Gaithersburg, MD (US); Carol Patricia MORENO QUINN, San Francisco, CA (US); Kevin James WOOLLARD, San Francisco, CA (US); Asha SETH, San Francisco, CA (US); Elena LIARTE MARIN, San Francisco, CA (US); Barbara MUSIAL, Gaithersburg, MD (US); Emma Suzanne COHEN, Cambridge (GB); Sam STRICKSON, Cambridge (GB); Kirsty HOUSLAY, Cambridge (GB)

(21) Appl. No.: 17/755,604

(22) PCT Filed: Nov. 3, 2020
(86) PCT No.: PCT/EP2020/080837

§ 371 Date: May 3, 2022

Related U.S. Application Data
(60) Provisional application No. 63/068,601, filed on Aug. 21, 2020.
(60) Provisional application No. 62/930,179, filed on Nov. 4, 2019.

(51) Int. Cl. C07K 16/24; C07K 16/28

(52) U.S. Cl. CPC C07K 16/244; C07K 16/2863; C07K 2317/76; C07K 2317/92; C07K 2317/51; C07K 2317/515

(57) ABSTRACT

present disclosure relates to a method of treating kidney injury, by administering an anti-IL-33 therapeutic agent which inhibits both ST2 signaling and RAGE signaling.

This application claims priority to U.S. Provisional Patent Application No. 62/930,179, filed Nov. 4, 2019 and U.S. Provisional Patent Application No. 63/068,601, filed Aug. 21, 2020. The content of these applications is incorporated herein by reference in its entirety.

### TECHNICAL FIELD

The present disclosure relates to a method of treating kidney injury, such as diabetic kidney disease.

### BACKGROUND

Chronic kidney disease (CKD) is a worldwide public health problem (Ritz et al. 1999; Nwankwo et al. 2005) that is associated with significant morbidity and mortality (Brenner et al. 2001; Lewis et al. 2001; Go et al. 2004). Diabetes accounts for approximately 45% of the incidence of second stage renal disease cases in the United States, with approximately 90% of these cases in patients with type 2 diabetes (USRDS 2009). The accepted standard of care for treatment of diabetic kidney disease (DKD) is the use of angiotensin converting enzyme inhibitors (ACEi) or angiotensin receptor blockers (ARB). Other pathways, such as RAGE signalling and signalling via the IL-33/ST2 axis, have emerged as contributors to disease progression. These pathways are mediated by the immune system through infiltrating immune cells and pro-inflammatory cytokines, chemokines and adhesion molecules (Hickey 2018; Ferhat et al JASN 2018 29:1272-1288). The complex pathophysiology of DKD (Brenner et al. 2001; Lewis et al. 2001) means that the hemodynamic effects of ACEi and ARB offer incomplete protection from the progressive loss of kidney function.

Thus, in this field there is an unmet medical need.

### SUMMARY OF THE DISCLOSURE

As shown in the examples and for the reasons set forth below, inhibiting signaling through both the ST2 receptor and RAGE provides for an effective treatment for kidney injury. The disclosure demonstrates for the first time that IL-33 mediates pathological signalling in different kidney cell types via distinct pathways. More specifically, a reduced form of IL-33 (redO:-33) is shown to initiate signalling in glomerular endothelial cells via the ST2 pathway. In addition, a hitherto unknown signalling pathway is described, in which oxidised IL-33 (oxIL-33) is shown to initiate signalling via RAGE/EGFR in kidney epithelium cellular sub-types. RAGE signalling has been implicated in kidney disease pathology, although oxIL-33 has not previously been recognised as a ligand for RAGE. Thus, the disclosure provides for a novel mechanism for treating kidney disease, by inhibiting oxIL-33 signalling. However, the treatment effect may not be limited to inhibition of oxIL-33, as binding and neutralising IL-33 can also inhibit pathological redIL-33 activity. Furthermore, the disclosure identifies that both isoforms of IL-33 have differential, potentially pathological effects on mesangial cells. RedTT 33 is shown to initiate production of inflammatory cytokines in this cell-type, whereas oxIL-33 induces mesangial cell proliferation. Mesangial expansion is a pathological hallmark of certain chronic kidney diseases, such as diabetic kidney disease (DKD). As such, the disclosure demonstrates the possibility of blocking multiple, distinct pathological pathways linked to kidney disease, by targeting a single cytokine, IL-33, in order to reduce or inhibit IL-33-mediated inflammation in the kidney, reduce or inhibit abnormal epithelial physiology associated with oxIL-33 signalling, and/or reduce or inhibit mesangial expansion

Therefore, a first aspect provides for a method of treating kidney injury, the method comprising administering an anti-IL-33 therapeutic agent which inhibits both ST2 signalling and RAGE signaling. In some embodiments, the method attenuates or inhibits activity of reduced IL-33 protein (redIL-33) and thereby inhibits ST2 signalling. In some embodiments, the method attenuates or inhibits activity of oxidised IL-33 protein (oxIL-33) and thereby inhibits RAGE signaling.

In some embodiments, the kidney injury comprises inflammation. In some embodiments, the kidney injury is inflammatory.

In some embodiments, the kidney injury is selected from diabetic kidney disease, fibrosis, glomerulonephritis (for example non-proliferative (such as minimal change glomerulonephritis, membrane glomerulonephritis, focal segmental glomerulosclerosis) or prolative (such as IgA nephropathy, membranoproliferative glomerulonephritis, post infectious glomerulonephritis, and rapidly progressive glomerulonephritis [such as Goodpastures syndrome and vasculitic disorders {which includes Wegners granulomatosis and microscopic polyangiitis}]), systemic lupus erythematosus, albuminuria, unilateral ureteral obstruction, Alport syndrome, polycystic kidney disease (PCKD), hypertensive glomerulosclerosis, chronic glomerulosclerosis, chronic obstructive uropathy, chronic tubulo-interstitial nephritis and ischemic nephropathy. In some embodiments, the kidney injury is diabetic kidney disease.

In some embodiments, the therapeutic agent is a chemical inhibitor or a binding molecule, such as an antibody or antigen-binding fragment thereof.

In some embodiments, wherein the therapeutic agent is an antibody or antigen-binding fragment thereof, it binds specifically to IL-33. In some embodiments, the antibody or antigen-binding fragment thereof specifically binds to redIL-33 and attenuates or inhibits activity of redIL-33, thereby inhibiting ST2 signalling. In some embodiments, the antibody or an antigen-binding fragment thereof binds to redIL-33 with a binding affinity of less than or equal to 100 pM, or less than or equal to 10 pM, for example less than or equal to 1 pM, such as 0.5 pM, in particular 0.05 pM (for example when measured using KinExA). In some embodiments, the antibody or an antigen-binding fragment thereof binds to redIL-33 with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. In some embodiments, the antibody or antigen binding fragment thereof binds to redIL-33 with an off rate (k(off)) of less than or equal to 5×10⁻¹ sec⁻¹, 10⁻¹ sec⁻¹, 5×10⁻² sec⁻¹, 10⁻² sec⁻¹, 5 ×10⁻³ sec⁻¹ or 10⁻³ sec⁻¹. Antibodies with these binding characteristics are particularly advantageous because they bind to and sequester the reduced form of IL-33, thereby enabling inhibition or attenuating activity of redIL-33. The strength of binding may also be sufficient to sequester redIL-33 prior to target engagement (i.e. prior to binding to ST-2). In addition, the strength of binding may also prevent the release of redIL-33 from the redIL-33/binding molecule complex, preventing conversion of red-IL-33 to the oxidised form. As such, these binding molecules or antigen-binding fragments therefore inhibit or attenuate the activity of oxIL-33, thereby inhibiting signalling via RAGE. Therefore, in some embodiments, the antibody or an antigen-binding fragment attenuates or inhibits the activity of oxIL-33 and thereby inhibits RAGE signaling.

In some embodiments, the antibody or antigen-binding fragment comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

In some embodiments, the antibody or antigen-binding fragment comprises a VH having the sequence of SEQ ID NO: 1 and a VL having the sequence of SEQ ID NO:19.

In another aspect, there is provided an anti-IL-33 therapeutic agent for use in a method of treating kidney injury in a subject, wherein the anti-IL-33 therapeutic agent is to be administered to the subject to attenuate or inhibit IL-33-mediated ST2 signalling and IL-33-mediated RAGE signaling.

In some embodiments, the IL-33-mediated RAGE signaling is IL-33-mediated RAGE-EGFR signaling. In some embodiments, the inhibition or attenuation of RAGE-EGFR signaling attenuates or inhibits RAGE-EGFR mediated effects. In some embodiments, the RAGE-EGFR mediated effect comprises abnormal epithelium physiology. In some embodiments, the abnormal epithelium physiology is abnormal epithelium remodelling. In some embodiments, the RAGE-EGFR mediated effect comprises abnormal mesangial expansion. In some embodiments, the abnormal mesangial expansion comprises abnormal mesangial cell proliferation.

In some embodiments, the inhibition or attenuation of ST2 signalling attenuates or inhibits ST2 mediated effects. In some embodiments, the ST2 mediated effect comprises abnormal inflammation in the kidney. In some embodiments, the abnormal inflammation comprises increased IL-4, IL-6, IL-8, IL-12, TNFa and/or IL1b secretion or expression, optionally increased IL-4, IL-6, IL-8 and/or IL-12 secretion or expression. In some embodiments, the abnormal inflammation comprises MAP kinase activation. In some embodiments, MAP kinase activation comprises p38 or JNK kinase activation. In some embodiments, the inflammation is in the endothelium, the glomeruli, or both.

In another aspect, there is provided a therapeutic agent that inhibits or attenuates the activity of reduced IL-33 to thereby inhibit or attenuate ST2 signaling, for use in the treatment of kidney injury, wherein the treatment further comprises inhibiting or attenuating the activity of oxidized IL-33 to thereby inhibit or attenuate RAGE signaling.

In another aspect, there is provided the use of a therapeutic agent that inhibits or attenuates the activity of reduced IL-33 to thereby inhibit or attenuate ST2 signaling, in the manufacture of a medicament for the treatment of kidney injury, wherein the treatment further comprises inhibiting or attenuating the activity of oxidized IL-33 to thereby inhibit or attenuate RAGE signaling.

In another aspect, there is provided a therapeutic agent that inhibits or attenuates the activity of oxidized IL-33 to thereby inhibit or attenuate RAGE signaling, for use in the treatment of kidney injury, wherein the treatment further comprises inhibiting or attenuating the activity of reduced IL-33 to thereby inhibit or attenuate ST2 signaling.

In another aspect, there is provided the use of a therapeutic agent that inhibits or attenuates the activity of oxidized IL-33 to thereby inhibit or attenuate RAGE signaling in the manufacture of a medicament for the treatment of kidney injury, wherein the treatment further comprises inhibiting or attenuating the activity of reduced IL-33 to thereby inhibit or attenuate ST2 signaling.

In another aspect, there is provided a therapeutic agent that inhibits or attenuates the activity of reduced IL-33 to thereby inhibit or attenuate ST2 signaling, and a therapeutic agent that inhibits or attenuates the activity of oxidized IL-33 to thereby inhibit or attenuate RAGE signaling, for use in the treatment of kidney injury.

In another aspect, there is provided the use of a therapeutic agent that inhibits or attenuates the activity of reduced IL-33 to thereby inhibit or attenuate ST2 signaling, and a therapeutic agent that inhibits or attenuates the activity of oxidized IL-33 to thereby inhibit or attenuate RAGE signaling, in the manufacture of a medicament for the treatment of kidney injury.

In another aspect, there is provided a therapeutic agent that inhibits or attenuates the activity of reduced IL-33 and oxidized IL-33 to thereby inhibit or attenuate ST2 signaling and RAGE signaling, for use in the treatment of kidney injury.

In another aspect, there is provided the use of a therapeutic agent that inhibits or attenuates the activity of reduced IL-33 and oxidized IL-33 to thereby inhibit or attenuate ST2 signaling and RAGE signaling, in the manufacture of a medicament for the treatment of kidney injury.

### BRIEF DESCRIPTION OF DRAWINGS

FIG. 1: Glomerular and tubular interstitial RNA expression of IL33 analysed from the ERCB cohort (A), the Ju 2013 cohort (B) and the Woroniecka cohort (C).

FIG. 2: (A) RNA expression of ST2 in normal and diabetic kidney and (B) RNA expression of ST2 is enriched in kidney cortex (steady state)

FIG. 3: Expression of IL33 mRNA in pre-clinical CKD mouse models

FIG. 4: Expression of RAGE mRNA in pre-clinical CKD mouse models

FIG. 5: Pre-clinical CKD model design using db/db UNX model with anti-ST2 and anti-RAGE interventions

FIG. 6: UACR changes in db/db UNX CKD model measured at 13 and 15 weeks in anti-ST2 and anti-RAGE treated mice compared to isotype control antibody treatment (NIP), shown as absolute values

FIG. 7: UACR changes in db/db UNX CKD model measured at 13 and 15 weeks in anti-ST2 and anti-RAGE treated mice compared to isotype control antibody treatment (MP), shown as a percent change at weeks 13 and 15 in comparison with week 10.

FIG. 8: Glomerular damage score (GDS) in db/db UNX pre-clinical CKD model when treated with anti-ST2 or isotype control antibody treatment (NIP)

FIG. 9: shows a grayscale heat map of the fold increase in kinases phosphorylation, compared to untreated control, for each of the detection assays on the MAP kinase phosphorylation antibody array. Reduced IL-33 (IL-33-01 and IL-33-16, respectively) did not cause any signals above baseline. oxIL-33 (oxidised IL-33-01) caused increased phosphorylation in multiple kinases;

FIG. 10: shows the signal pattern for each stimulation condition on a receptor tyrosine kinase (RTK) activity array. oxIL-33 but not reduced IL33-01 and IL33-16, respectively) triggered a positive signal on the RTK array corresponding to epidermal growth factor receptor (EGFR). Dot intensity correlates with receptor tyrosine kinase phosphorylation;

FIG. 11A: shows pEGFR (Tyr1068) activity in normal human bronchial epithelial (NEIBE) cells stimulated with increasing concentrations of IL-33 or EGFR ligands. oxIL-33, but not reduced IL-33 (IL33-01), promoted phosphorylation of the EGFR similarly to EGF, HB-EGF and TGFa;

FIG. 11B: shows pEGFR (Tyr1068) activity in A549 cells stimulated with increasing concentrations of IL-33 or EGFR ligands. oxIL-33 (oxidised IL-33-01), but not reduced IL-33 (IL-33-01) promoted phosphorylation of the EGFR similarly to EGF, HB-EGF and TGFa in a similar pattern to that seen in NHBE cells;

FIG. 11C: shows pEGFR (Tyr1068) activity in A549 cells stimulated with increasing concentrations of IL-33, EGFR ligands or RAGE ligands. oxIL-33, but not wild type (WT) IL-33 (IL-33-01), C->S mutated (mut) IL-33 (IL-33-16) or RAGE ligands, promoted phosphorylation of the EGFR similarly to EGF;

FIG. 12: shows that oxidised IL-33 induces the phosphorylation of multiple molecules involved in EGFR pathway (EGFR, PLC, AKT, JNK, ERK 1/2, p38) as analyzed by Western blot;

FIG. 13: shows STAT5 phosphorylation induced by oxIL-33 is reduced by increasing doses of anti-EGFR antibody as compared with isotype control;

FIG. 14: shows immunoprecipitation with anti-EGFR followed by detection of EGFR, RAGE or IL-33 by Western blot. IL-33 and RAGE co-precipitate with EGFR following NHBE stimulation with oxIL-33 suggesting that they form a complex. RAGE appears to be unique to the oxIL-33 signalling complex in comparison with EGF;

FIG. 15A: shows that oxIL-33 directly binds to RAGE. HMGB1 is a known RAGE ligand and acts as a positive control in this study;

FIG. 15B: show that oxIL-33 does not directly bind to EGFR (but the known EGFR ligand EGF does). However, when RAGE is added in to this assay in combination with oxIL-33 then EGFR binding is seen;

FIG. 16: shows immunoprecipitation with anti-EGFR or anti-RAGE, followed by western blot for EGFR, RAGE and IL-33 in wild type and RAGE-deficient A549 cells after activation with oxIL-33 at indicated time points;

FIG. 17: shows STAT5 phosphorylation induced by oxIL-33-01 is reduced by anti-RAGE antibody but not anti-ST2 antibody;

FIG. 18: shows (A) Primary proximal tubular epithelial cell (PTEC) secretion of IL33 in response to inflammatory mediators. PTEC response to exogenous IL-lb, but not exogenous redIL-33, detected by NFkB translocation, is also shown (B). (C) shows that PTEC do not respond to IL-33 in a dose-dependent manner by secreting the inflammatory cytokines IL-6, IL8, TNFa and IL1b. (D) shows that PTECs do not increase activation of p38 or JNK, two downstream mediators of ST2 signalling axis activation, above baseline levels, when treated with redIL-33. (E) shows that the detection of phosphorylated EGFR (pEGFR) increases in PTECs upon treatment with exogenous oxIL33 or EGF, but not redIL-33 or S1001A9 (RAGE ligand). Increased pEGFR in PTECs upon oxIL33 treatment is reduced in the presence of anti-RAGE and anti-EGFR antibodies (F). (G) shows that KIM-1 is increased in PTECs upon exposure to oxIL-33, but not reduced IL-33

FIG. 19: (A) shows primary glomerular endothelial cell (GEnC) secretion of IL33 increases in response to inflammatory mediators. (B) shows GEnC respond to exogenous redIL33 treatment by increasing NFkB translocation. Response is blocked in the presence of an anti-IL-33 antibody (C). (D) shows that GEnC secrete the inflammatory cytokines IL-6 and IL-8 upon stimulation with redIL-33 but not oxIL-33. The secretion of IL-8. TNFa, IL1b and IL-6 from GEnC upon treatment with redIL33 increases in a dose-dependent manner (E)

FIG. 20: (A) shows primary glomerular endothelial cell (GEnC) secrete inflammatory cytokines in response to IL33. Secretion is blocked in the presence of an anti-IL33 antibody. (B) shows that redIL-33 activates p38 and JNK kinase activity, which is inhibited in the presence of 33-640087_7B.

FIG. 21: (A) shows IL-33 signalling in human primary mesangial cells. Mesangial cells upregulate the level of IL-33 when stressed by interferon gamma and TNF alpha .Mesangial cells secrete IL-8 in a dose-dependent manner upon exposure to increasing concentrations of IL-33 (B). (C) shows that IL-8 secretion from mesangial cells is inhibited in the presence of 33-640087_7B. (D) shows that increasing concentrations of oxIL-33 increases mesangial cell proliferation.

FIG. 22A: shows relative wound healing density for A549 cells after treatment with reduced IL-33, oxIL-33 or EGF. Bar diagram shows mean and SEM from 6 technical replicates per condition;

FIG. 22B: shows relative wound healing density for NHBE cells after treatment with reduced IL-33, oxIL-33 or EGF. Bar diagram shows mean and SEM from 6 technical replicates per condition;

FIG. 23: shows percentage scratch wound closure of NHBE cells treated with media alone (unstimulated control), reduced IL-33, oxidised IL-33, or oxidised IL-33 in the presence of anti-ST2, anti-RAGE or anti-EGFR. Bar diagram shows mean and SEM from 6 technical replicates per condition;

### DETAILED DESCRIPTION

General Definitions

“Isolated” as employed herein refers to a protein in a non-natural environment in particular isolated from nature, for example the term does not include the protein in vivo, nor the protein in a sample taken from a human or animal body. Generally, proteins will be in a carrier such as a liquid or media, or may be formulated, frozen or freeze dried and all of these forms may be encompassed by “isolated” as appropriate. In one embodiment isolated does not refer to protein in a gel, for example a gel employed in Western blot analysis or similar.

‘IL-33’ protein as employed herein refers to interleukin 33, in particular a mammalian interleukin 33 protein, for example human protein deposited with UniProt number 095760. This entity is not a single species but instead exists as reduced and oxidized forms (Cohen et al Nature Comms). Given the rapid oxidation of the reduced form in vivo, for example in the period 5 minutes to 40 minutes, and in vitro, generally prior art references to IL-33 are in fact references to the oxidized form. The terms “IL-33” and “IL-33 polypeptide” and “IL-33 protein” are used interchangeably. In certain embodiments, IL-33 is full length. In another embodiment, IL-33 is mature, truncated IL-33 (amino acids 112-270). Recent studies suggest full length IL-33 is active (Cayrol and Girard, Proc Natl Acad Sci USA 106(22): 9021-6 (2009); Hayakawa et al., Biochem Biophys Res Commun. 387(1):218-22 (2009); Talabot-Ayer et al, J Biol Chem. 284(29): 19420-6 (2009)). However, N-terminally processed or truncated IL-33 including but not limited to aa 72-270, 79-270, 95-270, 99-270, 107-270, 109-270, 111-270, 112-270 may have enhanced activity (Lefrancais 2012, 2014). In another embodiment, IL-33 may include a full-length IL-33, a fragment thereof, or an IL-33 mutant or variant polypeptide, wherein the fragment of IL-33 or IL-33 variant polypeptide retains some or all functional properties of active IL-33.

Oxidized-IL-33, oxIL-33, IL-33-DSB (disulfide bonded) and DSB IL-33 are used interchangeably herein. Oxidized IL-33 refers to a protein visible as a distinct band, for example by western blot analysis under non-reducing conditions, in particular with a mass 4 Da less than the corresponding reduced form. In particular, it refers to a protein with one or two disulphide bonds between the cysteines independently selected from cysteines 208. 227, 232 and 259. Oxidised IL-33 refers to the form of IL-33 that binds to RAGE, and triggers RAGE-mediated signaling. In one embodiment the oxidized IL-33 shows no binding to ST2.

Reduced IL-33 and redIL-33 are employed interchangeably herein. Reduced IL-33 as employed herein refers to form of the IL-33 that binds to ST2 and triggers ST2 dependent signaling. In particular cysteines 208, 227, 232 and 259 of the reduced form are not disulfide bonded. An active fragment of redIL-33 as employed herein refers to a fragment with comparable activity to redIL-33, for example a similar extent of ST2-dependent signaling. In one embodiment an active fragment is 20, 30, 40, 50, 60, 70, 75, 80, 85, 90, 95, 96, 97, 98 or 99% of the activity of the full length redIL-33.

“ST2 signaling” as employed herein refers to the IL-33/ST2 system where IL-33 recognition by ST2 promotes dimerization with IL1 -RAcP on the cell surface and within the cell recruitment of receptor complex components MyD88, TRAF6 and IRAK1-4 to intracellular TIR domain. The ST2 receptor is expressed at baseline by Th2 cells, mast cells and other immune cell types, which can both be located in the kidney. The extracellular form of IL-33 stimulates target cells by binding to ST2 and subsequently activates NFκB and MAP Kinase pathways, leading to a range of functional responses including production of cytokines and chemokines. Thus ST2-dependent signalling may be interrupted by perturbing the interaction of IL-33 with ST2 or alternatively by interrupting the interaction with IL-1RAcP. ST-2 signaling “inhibition or attenuation” as employed herein refers to reducing or blocking signaling through the ST-2/IL-33 system. The extent of ST-2 signaling (and thus the inhibition or attenuation of it) can be determined by assaying concentration levels of inflammatory cytokines upregulated as a result of ST-2 signalling (e.g. IL-4, IL-6, IL-8 and IL-12). Concentrations of cytokines can be measured, for example using ELISA assays or quantitative mass spectrometry, from biological samples obtained from subjects undergoing the therapeutic methods described herein.

“RAGE signaling”, also referred to as “AGER signaling”, refers to the IL-33/RAGE system where IL-33 binds the receptor thereby generating pro-inflammatory gene activation. Inhibition or attenuation of RAGE signaling as employed herein, refers to reducing or blocking pathological signaling through the RAGE/IL-33 system, such as pro-inflammatory signalling, or signalling that induces abnormal epithelium remodelling or mesangial cell expansion in the glomeruli.

A “binding molecule” or “antigen binding molecule” employed in the present disclosure refers in its broadest sense to a molecule that specifically binds an antigenic determinant. In an embodiment, the binding molecule or antigen-binding fragment thereof specifically binds to IL-33, in particular redTT 33 and/or oxIL-33. In another embodiment, a binding molecule of the disclosure is an antibody or an antigen-binding fragment thereof.

“Antibody” as employed herein refers to an immunoglobulin molecule as discussed below in more detail, in particular a full-length antibody or a molecule comprising a full-length antibody, for example a DVD-Ig mole and the like.

A “binding fragment” or “antigen-binding fragment” is an epitope/antigen binding fragment of an antibody fragment, for example comprising a binding domain, in particular comprising 6 CDRs, such as 3 CDRs in heavy variable region and 3 CDRs in light variable region.

Unless specifically referring to full-sized antibodies such as naturally occurring antibodies, the term “anti-IL-33 antibodies” encompasses full-sized antibodies as well as antigen-binding fragments, variants, analogs, or derivatives of such antibodies, e.g., naturally occurring antibody or immunoglobulin molecules or engineered antibody molecules or fragments that bind antigen in a manner similar to antibody molecules.

The antibodies, or antigen-binding fragments, variants, or derivatives thereof employed herein may be described or specified in terms of the epitope(s) or portion(s) of an antigen, e.g., a target polypeptide disclosed herein (e.g., full length or mature IL-33) that they recognize or specifically bind. The portion of a target polypeptide that specifically interacts with the antigen binding domain of an antibody is an “epitope,” or an “antigenic determinant.” A target polypeptide may comprise a single epitope, but typically comprises at least two epitopes, and can include any number of epitopes, depending on the size, conformation, and type of antigen. Furthermore, it should be noted that an “epitope” on a target polypeptide may be or may include non-polypeptide elements, e.g., an epitope may include a carbohydrate side chain.

The minimum size of a peptide or polypeptide epitope for an antibody is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. A peptide or polypeptide epitope recognized by anti-IL-33 antibodies employed in the present disclosure may contain a sequence of at least 4, at least 5, at least 6, at least 7, more preferably at least 8, at least 9, at least 10, at least 15, at least 20, at least 25, or between about 15 to about 30 contiguous or non-contiguous amino acids of IL-33.

As used herein, the terms “treat” or “treatment” refer to both therapeutic treatment and prophylactic or preventative measures, wherein the object is to prevent or slow down (lessen) an undesired physiological change or disorder, such as the progression of an inflammatory condition. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans; domestic animals; farm animals; and zoo, sports, or pet animals such as dogs, cats, guinea pigs, rabbits, rats, mice, horses, cattle, cows, and so on. In one embodiment the patient is a human.

“Attenuates the activity of” as employed herein refers to reducing the relevant activity or stopping the relevant activity. Generally, attenuation and inhibition are employed interchangeably herein unless the context indicates otherwise.

Therapeutic Uses

As described herein, inhibiting signaling through both the ST2 receptor and RAGE may provide for an effective treatment for kidney injury. As defined herein, “kidney injury” refers to prolonged or chronic disease or injury of the kidney, for example from illness or trauma (including physical or chemical trauma). In other words, as used herein, “kidney injury” refers to a disease in which kidney function is chronically impaired and/or wherein kidney tissue is chronically damaged, for example, wherein these abnormalities persist for at least three months.

The methods described herein are relevant to the treatment of kidney injury mediated at least in part by IL-33. Levels of IL-33 have been shown to be increased locally in the kidney in subjects with kidney injury. Reduced IL-33 (redIL-33) stimulates inflammation in the kidney by activating signalling by directly binding to the receptor ST2. The disclosure identifies that IL-33-mediated ST2 takes place in multiple cell-types, including the endothelial cells and mesangial cells. The disclosure also describes the existence of a hitherto unknown IL-33 signalling pathway by which an oxidised form of IL-33 (oxIL-33) initiates signalling via RAGE/EGFR. The examples describe that RAGE-EGFR signalling is activated in the kidney epithelium. Surprisingly, the disclosure also identifies that oxIL-33-mediated RAGE/EGFR signalling includes increasing mesangial cell proliferation, potentially contributing to mesangial expansion observed in chronic diseases of the kidney, such as diabetic kidney disease. Therefore, the methods described herein are not only applicable to the treatment of IL-33-mediated inflammatory aspects of kidney disease, such as those mediated by ST2-signalling, but also to the treatment of elements of kidney disease mediated by oxIL-33 pathological signalling via RAGE/EGFR.

More specifically, dual blockade of IL-33-mediated ST2-dependent and RAGE-dependent signalling (such as RAGE-EGFR signalling) may provide improved outcomes for the treatment of subjects with kidney injury. In vivo modelling of kidney disease has shown a correlation between the levels of IL-33 and kidney damage. IL-33 in a reduced form signals via the well described ST2 signalling pathway. Signalling via the ST2 pathway generates inflammatory responses that contribute to the pathology of kidney injury and disease.

In addition, pathological signalling via RAGE has been associated with various kidney disorders (D'Agati et al Nat Rev Nephrol 2010:352-60). RAGE is a multi-ligand receptor of the IgG superfamily that binds to advance glycation end products. As such, antagonism of RAGE signalling has been put forward as a therapeutic strategy for the treatment of chronic kidney disease.

However, due to RAGE being a multiligand receptor (Fritz Trends in Biochemical Sciences 2011 36:625-632), direct inhibition of RAGE is likely to have off target effects and toxicity beyond any efficacy in kidney disease. By inhibiting a hitherto unknown ligand of RAGE in the context of kidney epithelium biology, it is believed that the therapeutic strategy disclosed herein is advantageous in comparison to the complete inhibition of RAGE. This is because it allows for the inhibition or attenuation of pathological RAGE signalling by directly inhibiting a RAGE ligand, oxIL-33. IL-33 expression is generally low in the serum of subjects with kidney injury (Bao et al. J Clin Immunol 2012:587-94; Caner et al.Renal Failure 2014:78-80; Musolino et al. Br. J. Haematol 2013:709-710 Mok et al. Rheumatology 2010:520-527). As such, targeting oxIL-33-RAGE-mediated signalling enables the inhibition or attenuation (i.e. dampening) of these elements of pathologic kidney injury, whilst potentially reducing off-target toxicities that may manifest by directly inhibiting RAGE. This is because the present disclosure enables the inhibition or attenuation of pathological RAGE signalling in a localised fashion, by targeting a RAGE ligand that is found predominantly at the site of disease, i.e. the kidney. Moreover, the strategy can be combined with inhibition of the redIL-33 ST2 pathway, allowing the inhibition and/or attenuation of two important pathological pathways involved in kidney injury.

As demonstrated in the examples, IL-33 kidney expression is elevated in multiple subjects with diabetic nephropathy, as well as in multiple pre-clinical models of kidney disease. The examples also demonstrate that both the ST2 and RAGE IL-33 signalling pathways are activated in the kidney epithelium, endothelium, and the glomeruli. The examples also show that inhibiting IL-33 signalling activity prevents the release of inflammatory mediators. Thus, the present disclosure provides a novel therapeutic strategy for the treatment of kidney injury, by inhibiting or attenuating both ST2 and RAGE dependent signalling mediated by IL-33.

As such, there is provided a method of treating kidney injury, the method comprising administering an anti-IL-33 therapeutic agent to a subject, wherein the anti-IL-33 therapeutic agent is administered to inhibit both ST2 signalling and RAGE signaling. Therapeutic agents are as defined elsewhere herein.

The present disclosure also shows for the first time that oxidised IL-33 binds to RAGE, which in turn complexes with EGFR. As such, the disclosure provides for the possibility of the use of therapeutic agents that can inhibit the signalling of oxidised IL-33 and thereby inhibit the potential oxIL-33-mediated pathological activation of RAGE. For example, the therapeutic agents may inhibit RAGE-EGFR complexing. The data disclosed herein demonstrate that preventing the formation of RAGE-EGFR complexes prevents IL-33-mediated RAGE/EGFR signalling, which may prevent tubular epithelial dysfunction induced by oxIL-33, and/or prevent mesangial dysfunction, such mesangial expansion, by inhibiting oxIL-33 mediated mesangial cell proliferation.

Therefore, the methods and therapeutic agents for use disclosed herein, in addition to inhibiting ST2 signalling, inhibit RAGE-EGFR signalling for the treatment of kidney injury.

In some instances, the therapeutic agent inhibits EGFR signalling. In some instances, the therapeutic agent inhibits RAGE-EGFR signalling. In some instances, the therapeutic agent inhibits oxidised-IL33-RAGE-EGFR signalling.

In some instances, the therapeutic agent inhibits binding of oxidised IL-33 to RAGE. In some instances, the therapeutic agent inhibits the formation of RAGE-EGFR complexes. In some instances, the therapeutic agent inhibits the formation of oxidised-IL33-RAGE-EGFR complexes.

In some instances, the therapeutic agent inhibits activation of EGFR. In some instances, the therapeutic agent inhibits phosphorylation of EGFR.

In some instances, the therapeutic agent inhibits RAGE-EGFR mediated effects. In some instances, the therapeutic agent inhibits effects mediated by the RAGE-EGFR complex. In some instances, the therapeutic agent inhibits effects mediated by the oxidised IL33-RAGE-EGFR complex.

In some instances, the therapeutic agent inhibits binding of oxidised IL-33 to RAGE, thereby inhibiting RAGE-EGFR complexing, thereby inhibiting RAGE-EGFR mediated effects such as downstream signalling.

In some instances, the therapeutic agent inhibits an IL-33-mediated EGFR effect. In some instances, the therapeutic agent inhibits IL-33-mediated EGFR signalling. In some instances, the therapeutic agent inhibits an oxidised IL-33-mediated EGFR effect. In some instances, the therapeutic agent inhibits oxidised IL-33-mediated EGFR signalling. In some instances, the therapeutic agent inhibits an oxidised IL-33-mediated RAGE-EGFR effect. Suitably the therapeutic agent inhibits oxidised IL-33-mediated RAGE-EGFR signalling.

In some instances, a RAGE-EGFR mediated effect is caused by the RAGE-EGFR complex, such as by the oxidised IL-33-RAGE-EGFR complex. Such effects may typically include downstream signalling which may be referred to herein as RAGE signalling, EGFR signalling or RAGE-EGFR signalling. In some instances, such signalling may include phosphorylation and/or chemokine release.

“RAGE-EGFR mediated effect” as recited herein refers to any physiological effect caused by the complexing of RAGE with EGFR in cell membranes and resulting aberrant EGFR activity. Such RAGE-EGFR mediated effects may present as abnormal kidney epithelium physiology. Abnormal kidney epithelium physiology may include negative effects on: barrier integrity; regulation and exchange of chemical entities between tissues and a cavity; secretion of chemicals into a cavity; maladaptive tissue repair; and/or tissue remodelling (for example, fibrosis).

In some instances, such RAGE-EGFR signalling includes phosphorylation of EGFR and subsequent phosphorylation of components in the EGFR pathway such as EGFR, PLC, JNK, MAPK/ERK 1/2, p38, and STAT5. Suitably EGFR signalling includes phosphorylation of tyrosine kinases such as JNK, MAPK/ERK, p38.

Therefore, in some instances, the therapeutic agent inhibits phosphorylation of components in the EGFR pathway. In some instances, the therapeutic agent inhibits phosphorylation of any one of: EGFR, PLC, JNK, MAPK/ERK 1/2, p38, and STAT5. In some instances, the therapeutic agent inhibits EGFR-mediated phosphorylation of any one of: EGFR, PLC, JNK, MAPK/ERK 1/2, p38, and STAT5. In some instances, therapeutic agent inhibits phosphorylation of tyrosine kinases. In some instances, the therapeutic agent inhibits phosphorylation of tyrosine kinases selected from: JNK, MAPK/ERK, p38. In some instances, the therapeutic agent inhibits EGFR-mediated phosphorylation of tyrosine kinases selected from: JNK, MAPK/ERK, and p38.

Therefore, in some instances, the therapeutic agent inhibits release of chemokines. In some instances, the therapeutic agent inhibits release of IL-8. In some instances, the therapeutic agent inhibits release of IL-4, IL-6, IL-8, IL-12, TNFa and/or IL1b. In some instances, the therapeutic agent inhibits EGFR-mediated release of chemokines. In some instances, the therapeutic agent inhibits EGFR-mediated release of IL-8.

In some instances, the RAGE-EGFR mediated effect may present as abnormal mesangial expansion. In some instances, the abnormal mesangial expansion comprises increased mesangial expansion. In some instances, the mesangial expansion comprises abnormal mesangial cell proliferation. In some instances, the abnormal mesangial cell proliferation comprises increased mesangial cell proliferation.

The methods and the therapeutic agents for use in the treatment or prevention of kidney injury.

The disclosure also provides for the use of any of the therapeutic agents as defined elsewhere herein in the manufacture of a medicine for the treatment of kidney injury.

The methods of the disclosure have been shown to reduce inflammatory burden in pre-clinical models of kidney disease. Furthermore, the present disclosure demonstrates that subjects with chronic kidney disease express elevated levels of interleukin-33. Accordingly, in some embodiments, the method comprises the treatment of kidney injury that is inflammatory. In some embodiments, the method comprises the treatment of kidney injury that comprises inflammation. In some embodiments, the method comprises the treatment of kidney injury that comprises chronic inflammation. In some embodiments, the method may treat or prevent inflammation associated with kidney injury. In some embodiments, the methods may treat or prevent acute inflammation associated with kidney injury. In some embodiments, the methods may treat or prevent chronic inflammation associated with kidney injury. In some embodiments, the method may be useful for the treatment of inflammatory conditions associated with kidney injury.

In some instances, the methods comprise inhibiting or attenuating IL-33-mediated ST2 signalling. In some instances, the IL-33-mediated ST2 signalling is redIL-33-mediated ST2 signalling. In some instances, inhibiting or attenuating IL-33-mediated ST2 signalling comprises inhibiting or attenuating an ST2-mediated effect.

In some instances, the ST2-mediated effect is abnormal inflammation in the kidney. In some instances, the abnormal inflammation in the kidney is increased inflammation in the kidney. In some instances, the abnormal inflammation in in the endothelium. In some instances, the abnormal inflammation is in the glomeruli. In some instances, the abnormal inflammation in the glomeruli is a result of mesangial cell stimulation. In some instances, the abnormal inflammation comprises increased IL-4, IL-6, IL-8, IL-12, TNFa and/or IL1b secretion or expression, optionally increased IL-4, IL-6, IL-8 and/or IL-12 secretion or expression. In some instances, the abnormal inflammation comprises increased IL-8 secretion or expression. In some instances, the abnormal inflammation comprises MAP kinase activation. In some instances, the MAP kinase activation comprises p38 or JNK kinase activation.

In some embodiments, the method described herein improves one or more symptoms associated with kidney injury. In some embodiments, the method may reduce weight loss, oedema, shortness of breath, fatigue, insomnia, cramps, nausea, itchy skin or headaches. In some embodiments, the method may improve appetite. Many of these symptoms may be linked to underlying impairment of kidney function associated with kidney injury. As such, improving kidney function by reducing kidney injury by performing the methods disclosed herein may improve any one or more of these symptoms.

As defined herein, “improve” means that the malaise of the subject with respect to one or more symptoms of a disease is lessened by performing the method described herein. The improvement of the subject may be ascertained by monitoring to assess the number of times a symptom manifests within the subject and to see how these occurrences reduce over time when the method is performed.

In some embodiments, the method described herein reduces the urine albumin:creatinine ratio (UACR) in a subject. For example, upon carrying out the method, the subject's UACR may be lowered. The UACR is the measure of the total albumin amount in a urine sample collected from the subject normalised to the concentration of creatinine. Higher UACR scores indicate that a subject has increased concentrations of albumin in urine (albuminuria). Albumin is normally released into the urine as a result of kidney injury. Therefore, the method can be used to lower the UACR score in a subject, wherein “lower” means that the UACR score is reduced during or after treatment in comparison the UACR prior to commencement of the therapy. The UACR score can be measured from urine samples collected from patients using any one of the numerous UACR tests available in the art

In some embodiments, the kidney injury is selected from diabetic kidney disease, fibrosis, glomerulonephritis (for example non-proliferative (such as minimal change glomerulonephritis, membrane glomerulonephritis, focal segmental glomerulosclerosis) or prolative (such as IgA nephropathy, membranoproliferative glomerulonephritis, post infectious glomerulonephritis, and rapidly progressive glomerulonephritis [such as Goodpastures syndrome and vasculitic disorders {which includes Wegners granulomatosis and microscopic polyangiitis}]), systemic lupus erythematosus, albuminuria, unilateral ureteral obstruction, Alport syndrome, polycystic kidney disease (PCKD), hypertensive glomerulosclerosis, chronic glomerulosclerosis, chronic obstructive uropathy, chronic tubulo-interstitial nephritis and ischemic nephropathy. All of these conditions have an associated inflammatory component, and may therefore benefit from treatment using the methods or therapeutic agents described herein.

In some embodiments, the method is for treating diabetic kidney disease. Diabetic kidney disease defined herein refers to a diagnosis of Type II Diabetes Mellitus and an estimated glomerular filtration rate (eGFR) of 30-75 ml/min. Typically, DKD is further defined as a diagnosis of UACR ratio of from 100 to 3000 mg albumin to g creatinine.

Tests for calculating eGFR are available in the art. Such tests typically take account of serum creatinine value, serum cystatin C value, age, gender and race. The calculation may also take account body surface adjustment values, such as height and/or mass. Typically, both the creatinine value and cystatin C value are standardized values. For example, the creatinine value is traceable to isotope dilution mass spectrometry (IDMS). The cystatin C value should be traceable to the International Federation of Clinical Chemistry and Laboratory Medicine (IFCC)/Institute for Reference Materials and Measurements (IRMM) working group. Typically, an eGFR of from 30-75 ml/min is indicative of a subject with mild to moderate-to-severe loss in kidney function. As such, the methods may be for use in the treatment or prevention of DKD with mild to moderate-to-severe loss in kidney function. In some embodiments, the methods are for improving kidney function in a subject with DKD.

The methods described herein may be performed in combination with known methods for the treatment of kidney injury. Known treatments for kidney injury, including for complications associated with kidney injury, include administration of 1) angiotensin converting enzyme (ACE) inhibitors, 2) statins, 3) diuretics, 4) erythropoietin, 5) iron supplements, 6) angiotensin-receptor blockers, 7) steroids, or 8) sodium-glucose transport protein-2 inhibitors (SGLT2i—also known as gliflozins).

Accordingly, the method may be for use in subject having undergone or undergoing therapy with an ACE inhibitor.

Accordingly, the method may be for use in subject having undergone or undergoing therapy with a statin.

Accordingly, the method may be for use in subject having undergone or undergoing therapy with a diuretic.

Accordingly, the method may be for use in subject having undergone or undergoing therapy with EPO.

Accordingly, the method may be for use in subject having undergone or undergoing therapy with an iron supplement.

Accordingly, the method may be for use in subject having undergone or undergoing therapy with and ARB.

Accordingly, the method may be for use in subject having undergone or undergoing therapy with a steroid.

Accordingly, the method may be for use in subject having undergone or undergoing therapy with an SGLT2i. In some instances, the SGLT2i is dagliflozin. Where the method comprises combined administration of another therapy, the methods of the disclosure encompass coadministration, using separate formulations or a single pharmaceutical formulation, and consecutive administration in either order. In some embodiments of the disclosure, the therapeutic agents described herein are administered in combination with anti-inflammatory drugs, wherein the therapeutic agent (e.g. the IL-33 antibody or antigen-binding fragment thereof) and the additional therapy may be administered sequentially, in either order, or simultaneously (i.e., concurrently or within the same time frame).

In some embodiments, the method is for treating a subject with elevated levels of IL-33 expression in the kidney (also referred to as “upregulated IL-33”). As described herein, subjects with kidney injury, for example subjects with diabetic nephropathy, have increased expression of IL-33 in the kidney glomeruli and tubulointerstitium. Therefore, the methods may be particularly beneficial for treating subject with kidney injury with upregulated IL-33. The methods may be particularly beneficial for treating subject with kidney injury with upregulated IL-33 in the glomeruli. The methods may be particularly beneficial for treating subject with kidney injury with upregulated IL-33 in the tubulointerstitium. The methods may be particularly beneficial for treating subject with kidney injury with upregulated IL-33 in the glomeruli and tubulointerstitium.

Methods for detecting IL-33 expression in cells are well known in the art and include, but are not limited to, PCR techniques, immunohistochemistry, flow cytometry, Western blot, ELISA, and the like. These methods can be employed to identify patients with upregulated IL-33.

In one embodiment, the method includes the application or administration of a therapeutic agent, e.g., an IL-33 antibody or antigen binding fragment thereof, to a subject or patient, or application or administration of the anti-IL-33 antibody or antigen binding fragment thereof to an isolated tissue or cell line from a subject or patient, where the subject or patient has kidney injury, a symptom of kidney injury, or a predisposition toward kidney injury. In another embodiment, the method is also intended to include the application or administration of a pharmaceutical composition comprising a therapeutic agent, e.g., the anti-IL-33 binding molecule, to a subject or patient, or application or administration of a pharmaceutical composition comprising the anti-IL-33 binding molecule to an isolated tissue or cell line from a subject or patient, who has kidney injury, a symptom of kidney injury, or a predisposition toward a disease.

In accordance with the methods of the present disclosure, at least one therapeutic agent, e.g., an anti-IL-33 binding molecule or antigen binding fragment thereof, as defined elsewhere herein is used to promote a positive therapeutic response with respect to an inflammatory response in kidney injury. By “positive therapeutic response” with respect to inflammation treatment is intended an improvement in the disease in association with the anti-inflammatory activity of these binding molecules, e.g., antibodies or fragments thereof, and/or an improvement in the symptoms associated with the disease. That is, an anti-inflammatory effect, the prevention of further inflammation and/or a reduction in existing inflammation, and/or a decrease in one or more symptoms associated with the disease can be observed. Thus, for example, an improvement in the disease may be characterized as a complete response. By “complete response” is intended an absence of clinically detectable disease with normalization of any previous test results. In one embodiment such a response must persist for at least one month following treatment according to the methods of the disclosure. Alternatively, an improvement in the disease may be categorized as being a partial response.

The methods of the present disclosure comprising the administration of at least one therapeutic agent, e.g., an anti-IL-33 binding molecule or antigen binding fragment thereof, may also find use in the treatment of inflammatory diseases and deficiencies or disorders of the immune system that are associated with IL-33 expressing cells, which are manifest as kidney injury. Inflammatory diseases are characterized by inflammation and tissue destruction, or a combination thereof. By “anti-inflammatory activity” is intended a reduction or prevention of inflammation. “Inflammatory disease” includes any inflammatory immune-mediated process where the initiating event or target of the immune response involves non-self antigen(s), including, for example, alloantigens, xenoantigens, viral antigens, bacterial antigens, unknown antigens, allergens or toxins.

In accordance with the methods of the present disclosure, at least one therapeutic agent, e.g., an anti-IL-33 binding molecule or antigen binding fragment thereof, is used to promote a positive therapeutic response with respect to treatment or prevention of an inflammatory kidney injury. By “positive therapeutic response” with respect to an inflammatory kidney injury is intended an improvement in the disease in association with the anti-inflammatory activity, or the like, of these antibodies, and/or an improvement in the symptoms associated with the disease. That is, a reduction in the inflammatory response including but not limited to reduced secretion of inflammatory cytokines, adhesion molecules, proteases, immunoglobulins, combinations thereof, and the like, increased production of anti-inflammatory proteins, a reduction in the number of autoreactive cells, an increase in immune tolerance, inhibition of autoreactive cell survival, reduction in apoptosis, reduction in endothelial cell migration, increase in spontaneous monocyte migration, reduction in and/or a decrease in one or more symptoms mediated by stimulation of IL-33-expressing cells can be observed. Such positive therapeutic responses are not limited to the route of administration.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, chromatography, and the like. In addition to these positive therapeutic responses, the subject undergoing therapy with the anti-IL-33 binding molecule, e.g., an antibody or antigen-binding fragment thereof, may experience the beneficial effect of an improvement in the symptoms associated with the disease.

A further embodiment of the disclosure is the use of anti-IL-33 binding molecule, e.g., antibodies or antigen binding fragments thereof, for diagnostic monitoring of protein levels in tissue as part of a clinical testing procedure, e.g., to determine the efficacy of a given treatment regimen. For example, detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

Therapeutic Agents

“Therapeutic agent” refers to an active pharmaceutical ingredient that is administered to a subject with the aim of having a beneficial effect on the disease state of a subject. As used herein, a therapeutic agent is an active ingredient designed for administration to a subject for the treatment or prevention of kidney injury. Kidney injury is as defined elsewhere herein. More specifically, the therapeutic agent(s) are designed to inhibit ST2 signalling, RAGE signaling, or both, for the treatment of kidney injury. In some embodiments, the one or more active ingredient(s) attenuate or inhibit activity of reduced IL-33 protein (redIL-33), thereby inhibiting ST2 signalling. In some embodiments, the one or more active ingredient(s) attenuate or inhibit activity of oxidised IL-33 protein (oxIL-33), thereby inhibiting RAGE signalling. The advantage of inhibiting both these signalling pathways in the context of treating kidney injury is described elsewhere herein.

In some embodiments, the one or more therapeutic agents comprise a “chemical inhibitor”. As employed herein, “chemical inhibitor” refers to a synthetic or semi-synthetic molecule with inhibitor activity, for example wherein the molecule has a molecular weight of 500 or less.

The chemical inhibitor may be designed to inhibit ST-2 signalling, RAGE signalling or both. In some embodiments, the chemical inhibitor is for inhibiting ST-2 signalling. The chemical inhibitor may inhibit ST-2 signalling by directly binding to ST-2 and antagonising signalling activity of ST-2. This could be achieved by binding to ST-2 to prevent binding of the ST-2 activating ligand redIL-33. Alternatively, the chemical inhibitor may bind directly to redIL-33 and inhibit binding to ST-2.

In some embodiments, the chemical inhibitor may inhibit RAGE signalling. The chemical inhibitor may inhibit RAGE signalling by directly binding to RAGE and antagonising signalling activity of RAGE. In some embodiments, the chemical inhibitor may attenuate or inhibit activity of oxIL-33 and thereby inhibits RAGE signaling. Alternatively, the chemical inhibitor may bind directly to oxIL-33 and inhibit binding to RAGE

In some embodiments, the chemical inhibitor may inhibit ST-2 signalling and RAGE signalling. This may be achieved, for example, by binding to an interface on IL-33 that engages with both ST-2 and RAGE in order to activate signalling.

In one embodiment the one or more therapeutic agent(s) comprise a binding molecule. Suitably, the binding molecule is an antibody or antigen-binding fragment, variant, or derivative thereof. Antibody or antigen binding fragment is as described elsewhere herein.

Suitably, the binding molecule specifically binds to IL-33. Such a binding molecule is also referred to as an “IL-33 binding molecule” or an “anti-IL-33 binding molecule”. Suitably, the binding molecule specifically binds to IL-33 and inhibits or attenuates IL-33 activity, for example, inhibits or attenuates reduced IL-33 activity, oxidised IL-33 activity or the activity of both.

Suitably the IL-33 binding molecule binds specifically to reduced IL-33, oxidised IL-33 or both reduced IL-33 and oxidised IL-33.

Suitably, the binding molecule may attenuate or inhibit IL-33 activity by binding IL-33 in reduced or oxidised forms. Suitably, wherein the binding molecule inhibits or attenuates reduced IL-33 activity and oxidised IL-33 activity, this is achieved by binding to IL-33 in reduced form (i.e. by binding to reduced IL-33).

Suitably, the binding molecule inhibits or attenuates the activity of both redIL-33 and oxIL-33, thereby inhibiting or attenuating both ST2 signalling and RAGE signalling.

Suitably, the inhibition of the activity of oxidised IL-33 down-regulates or turns off RAGE dependent signalling and/or RAGE mediated effects. Suitably, the inhibition down-regulates or turns off RAGE-EGFR dependent signalling and/or RAGE-EGFR mediated effects. Suitably, the inhibition down-regulates or turns off EGFR dependent signalling. Suitably, the inhibition down-regulates or turns off EGFR mediated effects. In particular, it has been shown that IL33 antagonists that bind to reduced IL-33 can prevent binding of oxidised IL-33 to RAGE, thereby inhibiting RAGE-EGFR signalling.

Suitably, the inhibition of the activity of oxidised IL-33 down-regulates or prevents RAGE-EGFR complexing. Suitably the inhibition down-regulates or prevents EGFR activation, suitably RAGE mediated EGFR activation.

Suitably, the binding molecule or a fragment or variant thereof may specifically bind to redIL-33 with a binding affinity (Kd) of less than 5×10⁻² M, 10⁻² M, 5×10⁻³ M, 10⁻³ M, 5×10⁻⁴ M, 10⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M. Suitably, the binding affinity to redIL-33 is less than 5×10⁻¹⁴ M (i.e. 0.05 pM). Suitably, the binding affinity is as measured using Kinetic Exclusion Assays (KinExA) or BIACORE™, suitably using KinExA, using protocols such as those described in WO2016/156440 (see e.g., Example 11), which is hereby incorporated by reference in its entirety. Binding molecules that bind to redIL-33 with this binding affinity appear to bind tightly enough to redIL-33 to prevent dissociation of the binding molecule/redIL-33 complex within biologically relevant timescales. Without wishing to be bound by theory, this binding strength is though to prevent release of the antigen prior to degradation of the antibody/antigen complex in vivo, such that redIL-33 is not released and cannot undergo conversion from redIL-33 to oxIL-33. Thus, when binding to redIL-33 with this binding affinity, the binding molecule can inhibit or attenuate the activity of oxIL-33 by preventing its formation, thereby inhibiting RAGE signalling.

In some instances, the binding molecule or a fragment thereof may specifically bind to redIL-33 with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. For example, a binding molecule of the disclosure may bind to redIL-33 or a fragment or variant thereof with an on rate (k(on)) greater than or equal to 10⁵M⁻¹ sec⁻¹, 5×10⁵M⁻¹ sec⁻¹, 10⁶ M⁻¹ sec⁻¹, or 5×10⁶ M⁻¹ sec⁻¹ or 10⁷M⁻¹sec⁻¹. Suitably, the k(on) rate is greater than or equal to 10⁷ M⁻¹ sec⁻i

In some instances, the binding molecule or a fragment thereof may specifically bind to redIL-33 with an off rate (k(off)) of less than or equal to 5×10⁻¹ sec⁻¹, 10⁻¹ sec⁻¹, 5×10′ sec⁻¹, 10⁻² sec⁻¹, 5×10⁻³ sec⁻¹ or 10⁻³ sec⁻¹. For example, a binding molecule of the disclosure may be said to bind to redIL-33 or a fragment or variant thereof with an off rate (k(off)) less than or equal to 5×10⁻⁴ sec⁻¹, 10⁴ sec⁻¹, 5×10⁻⁵ sec⁻¹, or 10⁻⁵ sec⁻¹, 5×10′ sec⁻¹, 10′ sec⁻¹, 5×10⁻⁷ sec⁻¹ or 10⁻⁷ sec⁻¹. Suitably, the k(off) rate is less than or equal to 10⁻³ sec⁻¹. IL-33 is an alarmin cytokine released rapidly and in high concentrations in response to inflammatory stimuli. redIL-33 is converted to the oxidised approximately 5-45 mins after release into the extracellular environment. Thus, to prevent conversion of redIL-33 to oxIL-33 the binding molecules described herein may bind to redIL-33 with these k(on) and/or k(off) rates. Without wishing to be bound by theory, these k(on)/k(off) rates are thought to ensure that the binding molecule can bind rapidly to redIL-33 before it converts to oxIL-33, thereby reducing the formation of oxIL-33, thereby attenuating or inhibiting RAGE signalling.

Suitably, the IL-33 binding molecule may competitively inhibit binding of IL-33 to any of the binding molecules referenced in Table 1:

TABLE 1
Exemplary anti-IL-33 antibody VH and VL pairs

| | SEQ ID | HCVR amino acid | | LCVR amino acid |
|---|---|---|---|---|
| Pair | NO: | sequence | SEQ ID NO: | sequence |
| 1 | SEQ ID NO: | EVQLLESGGGLVQPGGS | SEQ ID NO: | SYVLTQPPSVSVSPGQ |
| | 1 | LRLSCAASGFTFSSYAM | 19 | TASITCSGEGMGDKYA |
| | | SWVRQAPGKGLEWVSG | | AWYQQKPGQSPVLVI |
| | | ISAIDQSTYYADSVKGR | | YRDTKRPSGIPERFSGS |
| | | FTISRDNSKNTLYLQMN | | NSGNTATLTISGTQAM |
| | | SLRAEDTAVYYCARQK | | DEADYYCGVIQDNTG |
| | | FMQLWGGGLRYPFGY | | VFGGGTKLTVL |
| | | WGQGTMVTVSS | | |
| 2 | SEQ ID NO: | EVQLVESGGGLVQPGGS | SEQ ID NO: | DIQMTQSPSSVSASVG |
| | 2 | LRLSCAASGFTFRSFAM | 20 | DRVTITCRASQGFSSW |
| | | SWVRQAPGKGLELVSD | | LAWYQQKPGKAPKLLI |
| | | LRTSGGSTYYADSVKGR | | YAASSLQSGVPSRFSG |
| | | LTISRDNSKNTLYLQMN | | SGSGTDFTLTITNLQPE |
| | | SLRAEDTAVYYCAKSH | | DFATYYCQQANSFPLT |
| | | YSTSWFGGFDYWGQGT | | FGGGTKVEIK |
| | | LVTVSS | | |
| 3 | SEQ ID NO: | QVQLQESGPGLVKPSET | SEQ ID NO: | DIQMTQSPSSVSASVG |
| | 3 | LSLTCTVSGGSISSYYWS | 21 | DRVTITCRASQGISTW |
| | | WIRQPPGKGLELIGYIYY | | LAWFQQKPGKAPKLLI |
| | | SGSTNYNPSLKSRVTISV | | YAASTLQGGVPSRFSG |
| | | DTSKNHFSLKLSSVTAA | | SGSGPEFTLTISSLQPE |
| | | DTAVYYCARSQYTSSW | | DFATYYCQQANSFPW |
| | | YGSFDIWGQGTMVTVS | | TFGQGTKVEIK |
| | | S | | |
| 4 | SEQ ID NO: | QVQLVQSGAEVKKPGA | SEQ ID NO: | DIQMTQSPSSVSASVG |
| | 4 | SVKVSCKASGYTFNSYG | 22 | DRVTITCRASQGFSSW |
| | | ISWVRQAPGQGLEWMG | | LAWYQQKPGKAPQLLI |
| | | WISSHNGNSHYVQKFQ | | YAASSLQSGVPSRFSG |
| | | GRVSMTTDTSTSTAYM | | SGSGSDFTLTISSLQPE |
| | | ELRSLRSDDTAVYYCAR | | DFATYYCQQANSFPLT |
| | | HSYTTSWYGGFDYWGQ | | FGGGTKVEIK |
| | | GTLVTVSS | | |
| 5 | SEQ ID NO: | EVQLVESGGGLVQPGGS | SEQ ID NO: | DIQMTQSPSSVSASVG |
| | 5 | LRLSCAASGFTFSSYALT | 23 | DRVTITCRASQGVVSW |
| | | WVRQAPGKGLEWVSFI | | LAWYQQKPGKAPKLLI |
| | | SGSGGRPFYADSVKGRF | | YAASSLQSGVPSRFSG |
| | | TISRDNSKNMLYLQMNS | | SGSGTDFTLTISSLQPE |
| | | LRAEDTAIYYCAKSLYT | | DFATYYCQQSNSFPFT |
| | | TSWYGGFDSWGQGTLV | | LGPGTKVDIK |
| | | TVSS | | |
| 6 | SEQ ID NO: | EVQLVESGGGLVQPGGS | SEQ ID NO: | DIQMTQSPSSVSASVG |
| | 6 | LRLSCAASGFTFSNYAM | 24 | DRVTITCRASQGISSWL |
| | | TWVRQAPGKGLEWVST | | AWYQQKPGKAPQLLI |
| | | ISGSGDNTYYADSVQGR | | YAASRLQSGVPSRFWG |
| | | FTISRGHSKNTLYLQMN | | SGSGTDFTLTISSLQPE |
| | | SLRAEDTAVYYCAKPT | | DFATYYCQQANNFPFT |
| | | YSRSWYGAFDFWGQGT | | FGPGTKVDIK |
| | | MVTVSS | | |
| 7 | SEQ ID NO: | EVQLVESGGNLEQPGGS | SEQ ID NO: | DIQMTQSPSSVSASVG |
| | 7 | LRLSCTASGFTFSRSAM | 25 | DRVTITCRASQGIFSWL |
| | | NWVRRAPGKGLEWVSG | | AWYQQKPGKAPKLLI |
| | | ISGSGGRTYYADSVKGR | | YAASSLQSGVPSRFSG |
| | | FTISRDNSKNTLYLQMN | | SGSGTDFTLTISSLQPE |
| | | SLSAEDTAAYYCAKDS | | DFAIYYCQQANSVPITF |
| | | YTTSWYGGMDVWGHG | | GQGTRLEIK |
| | | TTVTVSS | | |
| 8 | SEQ ID NO: | EVQLLESGGGLVQPGGS | SEQ ID NO: | QSVLTQPPSASGTPGQ |
| | 8 | LRLSCAASGFTFSDYYM | 26 | RVTISCTGSSSNIGAVY |
| | | NWVRQAPGKGLEWVSS | | DVHWYQQLPGTAPKL |
| | | ISRYSSYIYYADSVKGRF | | LIYRNNQRPSGVPDRF |
| | | TISRDNSKNTLYLQMNS | | SGSKSGTSASLAISGLR |
| | | LRAEDTAVYYCARDIG | | SEDEADYYCQTYDSSR |
| | | GMDVWGQGTLVTVSS | | WVFGGGTKLTVL |
| 9 | SEQ ID NO: | EVQLLESGGGLVQPGGS | SEQ ID NO: | QSVLTQPPSASGTPGQ |
| | 9 | LRLSCAASGFTFSNYYM | 27 | RVTISCSGSSSNIGNNA |
| | | HWVRQAPGKGLEWVSS | | VSWYQQLPGTAPKLLI |
| | | ISARSRYHYYADSVKGR | | YASNMRVIGVPDRFSG |
| | | FTISRDNSKNTLYLQMN | | SKSGTSASLAISGLRSE |
| | | SLRAEDTAVYYCARLA | | DEADYYCGAWDDSQK |
| | | TRHNAFDIWGQGTLVT | | ALVFGGGTKLTVL |
| | | VSS | | |
| 10 | SEQ ID NO: | EVQLLESGGGLVQPGGS | SEQ ID NO: | QSVLTQPPSASGTPGQ |
| | 10 | LRLSCAASGFTFSNYYM | 28 | RVTISCSGSSSNIGRNA |
| | | HWVRQAPGKGLEWVSS | | VNWYQQLPGTAPKLLI |
| | | ISARSSYIYYADSVKGRF | | YASNMRVSGVPDRFS |
| | | TISRDNSKNTLYLQMNS | | GSKSGTSASLAISGLRS |
| | | LRAEDTAVYYCARLAT | | EDEADYYCWAWDDS |
| | | RNNAFDIWGQGTLVTV | | QKVGVFGGGTKLTVL |
| | | SS | | |
| 11 | SEQ ID NO: | EVQLLESGGGLVQPGGS | SEQ ID NO: | QSVLTQPPSASGTPGQ |
| | 11 | LRLSCAASGFTFSRYYM | 29 | RVTISCSGSSSNIGRNA |
| | | HWVRQAPGKGLEWVSS | | VNWYQQLPGTAPKLLI |
| | | ISAQSSHIYYADSVEGRF | | YASNMRRSGVPDRFSG |
| | | TISRDNSKNTLYLQMNS | | SKSGTSASLAISGLRSE |
| | | LRAEDTAVYYCARLAT | | DEADYYCSAWDDSQK |
| | | RQNAFDIWGQGTLVTV | | VVVFGGGTKLTVL |
| | | SS | | |
| 12 | SEQ ID NO: | EVQLLESGGGLVQPGGS | SEQ ID NO: | QSVLTQPPSASGTPGQ |
| | 12 | LRLSCAASGFTFSNYYM | 30 | RVTISCSGSSSNIGNNA |
| | | HWVRQAPGKGLEWVSS | | VNWYQQLPGTAPKLLI |
| | | ISARSSYLYYADSVKGR | | YASNMRRPGVPDRFSG |
| | | FTISRDNSKNTLYLQMN | | SKSGTSASLAISGLRSE |
| | | SLRAEDTAVYYCARLA | | DEADYYCEAWDDSQK |
| | | TRHVAFDIWGQGTLVT | | AVVFGGGTKLTVL |
| | | VSS | | |
| 13 | SEQ ID NO: | MRAWIFFLLCLAGRALA | SEQ ID NO: | MRAWIFFLLCLAGRAL |
| | 13 | QVQLMQSGAEVKKPGA | 31 | ADIQLTQSPSFLSASVG |
| | | SVKVSCKASGYTFTSY | | DRVTITCKASQDVGTA |
| | | WMHWVRQAPGQGLEW | | VAWYQQKPGKAPKLL |
| | | MGTIYPRNSNTDYNQKF | | IYWASTRHTGVPSRFS |
| | | KARVTMTRDTSTSTVY | | GSGSGTEFTLTISSLQP |
| | | MELSSLRSEDTAVYYCA | | EDFATYYCQQAKTYPF |
| | | RPLYYYLTSPPTLFWGQ | | TFGSGTKLEIKR |
| | | GTLVTVSS | | |
| 14 | SEQ ID NO: | EVQLVETGGGLIQPGGS | SEQ ID NO: | EIVLTQSPGTLSLSPGE |
| | 14 | LRLSCAASGFTFSSYAM | 32 | RATLSCRASQSVGINLS |
| | | SWVRQAPGKGLEWVSA | | WYQQKPGQAPRLLIY |
| | | ISGSGGSTYYADSVKGR | | GASHRATGIPDRFSGS |
| | | FTISRDNSKNTLYLQMN | | GSGTDFTLTISRLEPED |
| | | SLRAEDTAVYYCARTL | | FAVYYCHQYSQSPPFT |
| | | HGIRAAYDAFIIWGQGT | | FGGGTKVEIK |
| | | LVTVSS | | |
| 15 | SEQ ID NO: | EVQLVETGGGLIQPGGS | SEQ ID NO: | EIVLTQSPGTLSLSPGE |
| | 15 | LRLSCAASGFTFSFYAM | 33 | RATLSCRASQSVGINLS |
| | | SWVRQAPGKGLEWVSA | | WYQQKPGQAPRLLIY |
| | | ISGSGGSTYYADSVKGR | | GASHRLTGIPDRFSGSG |
| | | FTISRDNSKNTLYLQMN | | SGTDFTLTISRLEPEDF |
| | | SLRAEDTAVYYCARTL | | AVYYCHQYSQPPPFTF |
| | | HGIRAAYDAFIIWGQGT | | GGGTKVEIK |
| | | LVTVSS | | |
| 16 | SEQ ID NO: | EVQLVETGGGLIQPGGS | SEQ ID NO: | EIVLTQSPGTLSLSPGE |
| | 16 | LRLSCAASGFTFSFYAM | 34 | RATLSCRASQSVGINLS |
| | | SWVRQAPGKGLEWVSA | | WYQQKPGQAPRLLIY |
| | | ISGSGGSTYYADSVKGR | | GASHRLTGIPDRFSGSG |
| | | FTISRDNSKNTLYLQMN | | SGTDFTLTISRLEPEDF |
| | | SLRAEDTAVYYCARTIH | | AVYYCHQYSQPPPFTF |
| | | GIRAAYDAFIIWGQGTL | | GGGTKVEIK |
| | | VTVSS | | |
| 17 | SEQ ID NO: | EVQLVESGGGLVQPGGS | SEQ ID NO: | DIQMTQSPSSLSASVG |
| | 17 | LRLSCAASGFTFSSYWM | 35 | DRVTITCKASQNINKH |
| | | YWVRQAPGKGLEWVA | | LDWYQQKPGKAPKLLI |
| | | AITPNAGEDYYPESVKG | | YFTNNLQTGVPSRFSG |
| | | RFTISRDNAKNSLYLQM | | SGSGTDFTLTISSLQPE |
| | | NSLRAEDTAVYYCARG | | DFATYYCFQYNQGWT |
| | | HYYYTSYSLGYWGQGT | | FGGGTKVEIK |
| | | LVTVSS | | |
| 18 | SEQ ID NO: | EVQLVESGGGLVQPGGS | SEQ ID NO: | EIVLTQSPATLSLSPGE |
| | 18 | LRLSCAASGFTFSSFSMS | 36 | RATLSCRASESVAKYG |
| | | WVRQAPGKGLEWVATI | | LSLLNWFQQKPGQPPR |
| | | SGGKTFTDYVDSVKGRF | | LLIFAASNRGSGIPARF |
| | | TISRDDSKNTLYLQMNS | | SGSGSGTDFTLTISSLE |
| | | LRAEDTAVYYCTRANY | | PEDFAVYYCQQSKEVP |
| | | GNWFFEVWGQGTLVTV | | FTFGQGTKVEIK |
| | | SS | | |

All these binding molecules have been reported to bind to IL-33 and inhibit or attenuate ST-2 signalling. Thus, a binding molecule or binding fragment thereof that competes for binding to redIL-33 with any of the antibodies disclosed in Table 1 may inhibit or attenuate ST-2 signalling.

A binding molecule or fragment thereof is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether a binding molecule or fragment thereof competes for binding to redIL-33 by using an in vitro competitive binding assay, such as the HTRF assay described below in detail in the examples. For example, the skilled person could label a recombinant antibody of Table 1 with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding molecules the skilled person could first mix various concentrations of a test binding molecule with a fixed concentration of the labelled antibody of Table 1. A reduction in the FRET signal when the mixture is incubated with labelled IL-33 in comparison with a labelled antibody-only positive control would indicate competitive binding to IL-33. A binding molecule or fragment thereof may be said to competitively inhibit binding of the reference antibody to a given epitope by at least 90%, at least 80%, at least 70%, at least 60%, or at least 50%.

Suitably, the IL-33 binding molecule may competitively inhibit binding of IL-33 to the binding molecule 33 640087-7B (as described in WO2016/156440). Suitably, WO2016/156440 discloses that 33-640087-7B binds to redIL-33 with particularly high affinity and attenuates both ST-2 and RAGE-dependent IL-33 signalling. Thus, a binding molecule that competitively inhibits binding of IL-33 to the binding molecule 33_640087-7B is highly likely to inhibit both redIL-33 and oxIL-33 signalling and thus be particularly suitable for use in the methods described herein.

In some instances, the binding molecule that inhibits or attenuates IL-33 activity is selected from any of the following anti-IL-33 antibodies: 33_640087-7B (as described in WO2016/156440), ANB020 known as Etokimab (as described in WO2015/106080), 9675P (as described in US2014/0271658), A25-3H04 (as described in US2017/0283494), Ab43 (as described in WO2018/081075), IL33-158 (as described in US2018/0037644), 10C12.38.H6. 87Y.581 lgG4 (as described in WO2016/077381) or binding fragments thereof, each of the documents being incorporated herein by reference. All of these antibodies are referenced in Table 1.

Suitably, the binding molecule or antigen-binding fragment comprises the complementarity determining regions (CDRs) of a variable heavy domain (VH) and a variable light domain (VL) pair selected from Table 1. Pair 1 corresponds to the VH and VL domain sequences of 33_640087-7B described in WO2016/156440. Pairs 2-7 correspond to VH and VL domain sequences of antibodies described in US2014/0271658. Pairs 8-12 correspond to VH and VL domain sequences of antibodies described in US2017/0283494. Pair 13 corresponds to the VH and VL domain sequences of ANB020, described in WO2015/106080. Pairs 14-16 correspond to VH and VL domain sequences of antibodies described in WO2018/081075. Pair 17 corresponds to VH and VL domain sequences of IL33-158 described in US2018/0037644. Pair 18 corresponds to VH and VL domain sequences of 10C12.38.H6. 87Y.581 lgG4 described in WO2016/077381.

Suitably, the IL-33 binding molecule is an antibody or antigen-binding fragment comprising the complementarity determining regions (CDRs) of the heavy chain variable region (HCVR) comprising the sequence of SEQ ID NO:1 and the complementarity determining regions (CDRs) of light chain variable region (LCVR) comprising the sequence of SEQ ID NO:19. These CDRs correspond to those derived from 33 640087-7B (as described in WO2016/156440), which binds reduced IL-33 and inhibits its conversion to oxidised IL-33. 33 640087-7B is described in full in WO2016/156440 which is incorporated by reference herein. Thus, this antibody may be particularly useful in the methods described herein to inhibit or attenuate both ST-2 and RAGE signalling.

Suitably, the IL-33 binding molecule is an antibody or antigen-binding fragment comprising the complementarity determining regions (CDRs) of the heavy chain variable region (HCVR) comprising the sequence of SEQ ID NO:7 and the complementarity determining regions (CDRs) of light chain variable region (LCVR) comprising the sequence of SEQ ID NO:25. These CDRs correspond to those derived from the antibody 9675P. 9675P is described in full in US2014/0271658 which is incorporated by reference herein.

Suitably, the IL-33 binding molecule is an antibody or antigen-binding fragment comprising the complementarity determining regions (CDRs) of the heavy chain variable region (HCVR) comprising the sequence of SEQ ID NO:11 and the complementarity determining regions (CDRs) of light chain variable region (LCVR) comprising the sequence of SEQ ID NO:29. These CDRs correspond to those derived from the antibody A25-3H04. A25-3H04 is described in full in US2017/0283494 which is incorporated by reference herein.

Suitably, the IL-33 binding molecule is an antibody or antigen-binding fragment comprising the complementarity determining regions (CDRs) of the heavy chain variable region (HCVR) comprising the sequence of SEQ ID NO:13 and the complementarity determining regions (CDRs) of light chain variable region (LCVR) comprising the sequence of SEQ ID NO:31. These CDRs correspond to those derived from the antibody ANB020. ANB020 is described in full in WO2015/106080 which is incorporated by reference herein.

Suitably, the IL-33 binding molecule is an antibody or antigen-binding fragment comprising the complementarity determining regions (CDRs) of the heavy chain variable region (HCVR) comprising the sequence of SEQ ID NO:16 and the complementarity determining regions (CDRs) of light chain variable region (LCVR) comprising the sequence of SEQ ID NO:34. These CDRs correspond to those derived from the antibody Ab43. Ab43 is described in full in WO2018/081075 which is incorporated by reference herein.

Suitably, the IL-33 binding molecule is an antibody or antigen-binding fragment comprising the complementarity determining regions (CDRs) of the heavy chain variable region (HCVR) comprising the sequence of SEQ ID NO:17 and the complementarity determining regions (CDRs) of light chain variable region (LCVR) comprising the sequence of SEQ ID NO:35. These CDRs correspond to those derived from the antibody IL33-158. IL33-158 is described in full in US2018/0037644 which is incorporated by reference herein.

Suitably, the IL-33 binding molecule is an antibody or antigen-binding fragment comprising the complementarity determining regions (CDRs) of the heavy chain variable region (HCVR) comprising the sequence of SEQ ID NO:18 and the complementarity determining regions (CDRs) of light chain variable region (LCVR) comprising the sequence of SEQ ID NO:36. These CDRs correspond to those derived from the antibody 10C12.38.H6. 87Y.581 lgG4. 10C12.38.H6. 87Y.581 lgG4 is described in full in WO2016/077381 which is incorporated by reference herein.

Suitably the skilled person knows of available methods in the art to identify CDRs within the heavy and light variable regions of an antibody or antigen-binding fragment thereof. Suitably the skilled person may conduct sequence-based annotation, for example. The regions between CDRs are generally highly conserved, and therefore, logic rules can be used to determine CDR location. The skilled person may use a set of sequence-based rules for conventional antibodies (Pantazes and Maranas, Protein Engineering, Design and Selection, 2010), alternatively or additionally he may refine the rules based on a multiple sequence alignment. Alternatively, the skilled person may compare the antibody sequences to a publicly available database operating on Kabat, Chothia or IMGT methods using the BLASTP command of BLAST+to identify the most similar annotated sequence. Each of these methods has devised a unique residue numbering scheme according to which it numbers the hypervariable region residues and the beginning and ending of each of the six CDRs is then determined according to certain key positions. Upon alignment with the most similar annotated sequence, for example, the CDRs can be extrapolated from the annotated sequence to the non-annotated sequence, thereby identifying the CDRs. Suitable tools/databases are: the Kabat database, Kabatman, Scalinger, IMGT, Abnum for example.

Suitably, the IL-33 therapeutic agent is an antibody or antigen-binding fragment comprising a variable heavy domain (VH) and variable light domain (VL) pair selected from Table 1.

Suitably, the IL33 antibody or antigen binding fragment therefore comprises a VH domain of the sequence of SEQ ID NO:1 and a VL domain of the sequence of SEQ ID NO:19.

Suitably, the IL33 antibody or antigen binding fragment therefore comprises a VH domain of the sequence of SEQ ID NO:7 and a VL domain of the sequence of SEQ ID NO:25.

Suitably, the IL33 antibody or antigen binding fragment therefore comprises a VH domain of the sequence of SEQ ID NO:11 and a VL domain of the sequence of SEQ ID NO:29.

Suitably, the IL33 antibody or antigen binding fragment therefore comprises a VH domain of the sequence of SEQ ID NO:13 and a VL domain of the sequence of SEQ ID NO:31.

Suitably, the IL33 antibody or antigen binding fragment therefore comprises a VH domain of the sequence of SEQ ID NO:16 and a VL domain of the sequence of SEQ ID NO:34.

Suitably, the IL33 antibody or antigen binding fragment therefore comprises a VH domain of the sequence of SEQ ID NO:17 and a VL domain of the sequence of SEQ ID NO:35.

Suitably, the IL33 antibody or antigen binding fragment therefore comprises a VH domain of the sequence of SEQ ID NO:18 and a VL domain of the sequence of SEQ ID NO:36.

Suitably, therefore, the therapeutic agent is a binding molecule which may comprise 3 CDRs, for example in a heavy chain variable region independently selected from SEQ ID NO: 1, 7, 11, 13, 16, 17 and 18.

Suitably the IL-33 the therapeutic agent is a binding molecule which comprises 3 CDRs in a heavy chain variable region according to SEQ ID NO:1.

Suitably, the IL-33 therapeutic agent is a binding molecule which may comprise 3 CDRs in a light chain variable region independently selected from SEQ ID NO: 19, 25, 29, 31, 34, 35 and 36.

Suitably, the IL-33 therapeutic agent is a binding molecule which comprises 3 CDRs in a light chain variable region according to SEQ ID NO:19.

Suitably, therefore, the IL-33 therapeutic agent is a binding molecule which may comprise 3 CDRs, for example in a heavy chain variable region independently selected from SEQ ID NO: 1, 7, 11, 13, 16, 17 and 18 and 3 CDRs, for example in a light chain variable region independently selected from SEQ ID NO: 19, 25, 29, 31, 34, 35 and 36.

Suitably, therefore the IL-33 therapeutic agent is a binding molecule which comprises 3 CDRs in a heavy chain variable region according to SEQ ID NO:1, and 3 CDRs in a light chain variable region according to SEQ ID NO:19.

Suitably, therefore, the IL-33 therapeutic agent is a binding molecule which may comprise a variable heavy domain (VH) and a variable light domain (VL) having VH CDRs 1-3 having the sequences of SEQ ID NO: 37, 38 and 39, respectively, wherein one or more VHCDRs have 3 or fewer single amino acid substitutions, insertions and/or deletions.

Suitably, therefore, the IL-33 therapeutic agent is a binding molecule comprising a VH domain which comprises VHCDRs 1-3 of SEQ ID NO: 37, SEQ ID NO: 38 and SEQ ID NO: 39, respectively.

Suitably, therefore, the IL-33 therapeutic agent is a binding molecule comprising a VH domain which comprises VHCDRs 1-3 consisting of SEQ ID NO: 37, SEQ ID NO: 38 and SEQ ID NO: 39, respectively.

Suitably, therefore, the IL-33 therapeutic agent is a binding molecule which may comprise a variable heavy domain (VH) and a variable light domain (VL) having VL CDRs 1-3 having the sequences of SEQ ID NO: 40, 41 and 42, respectively, wherein one or more VLCDRs have 3 or fewer single amino acid substitutions, insertions and/or deletions.

Suitably, therefore, the IL-33 therapeutic agent is a binding molecule comprising a VL domain which comprises VLCDRs 1-3 of SEQ ID NO: 40, SEQ ID NO: 41 and SEQ ID NO: 42, respectively.

Suitably, therefore, the IL-33 therapeutic agent is a binding molecule comprising a VL domain which comprises VLCDRs 1-3 consisting of SEQ ID NO: 40, SEQ ID NO: 41 and SEQ ID NO: 42, respectively.

Suitably, therefore, the IL-33 therapeutic agent is a binding molecule which may comprise a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

Suitably, therefore the IL-33 therapeutic agent is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 1, 7, 11, 13, 16, 17 and 18.

Suitably, therefore the IL-33 therapeutic agent is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 1.

Suitably, therefore the IL-33 therapeutic agent is an antibody or binding fragment thereof comprising a VH and VL, wherein a VH disclosed above, has a sequence with 1, 2, 3 or 4 amino acids in the framework deleted, inserted and/or independently replaced with a different amino acid.

Suitably, therefore the IL-33 therapeutic agent is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO: 19, 25, 29, 31, 34, 35 and 36.

Suitably, therefore the IL-33 therapeutic agent is an antibody or binding fragment thereof comprising a VH and VL, wherein the VL has an amino acid sequence at least 90%, for example 91, 92, 93, 94, 95, 96, 97, 98, 99 or 100% identical to a VL according to SEQ ID NO: 19.

Suitably, therefore the IL-33 therapeutic agent is an antibody or binding fragment thereof comprising a VH and VL, wherein a VL disclosed above has a sequence with 1, 2, 3 or 4 amino acids in the framework independently deleted, inserted and/or replaced with a different amino acid.

Suitably, therefore the IL-33 therapeutic agent is an antibody or binding fragment thereof comprising a VH and VL, wherein the VH has an amino acid sequence at least 90%, for example 91, 92, 93, 94, 95, 96, 97, 98, 99 or 100% identical to a VH according to SEQ ID NO: 1, 7, 11, 13, 16, 17 and 18, and VL has an amino acid sequence at least 90%, for example 91, 92, 93, 94, 95, 96, 97, 98, 99 or 100% identical to a VL according to SEQ ID NO: 19, 25, 29, 31, 34, 35 and 36.

Suitably, therefore the IL-33 therapeutic agent is an antibody or binding fragment thereof comprising a VH and VL, wherein the VH has an amino acid sequence consisting of SEQ ID NO: 1, 7, 11, 13, 16, 17 and 18, and the VL has an amino acid sequence consisting of SEQ ID NO: 19, 25, 29, 31, 34, 35 and 36.

Suitably, therefore the IL-33 therapeutic agent is an antibody or binding fragment thereof comprising a VH and VL, wherein the VH has an amino acid sequence consisting of SEQ ID NO: 1, and the VL has an amino acid sequence consisting of SEQ ID NO: 19.

Suitably, the binding molecule may be selected from: an antibody, an antigen-binding fragment thereof, an aptamer, at least one heavy or light chain CDR of a reference antibody molecule, and at least six CDRs from one or more reference antibody molecules.

Suitably, the IL-33 therapeutic agent is an antibody or binding fragment thereof. Suitably, the IL-33 therapeutic agent is an anti-IL-33 antibody or binding fragment thereof. Suitably, the anti-IL-33 antibody or binding fragment thereof specifically binds to IL-33, in particular reduced IL-33 or oxidised IL-33.

Suitably the IL-33 therapeutic agent inhibits the activity of oxidised IL-33, suitably by inhibiting the formation of oxidised IL-33. Suitably the IL-33 therapeutic agent inhibits the conversion of reduced IL-33 into oxidised IL-33.

Suitably the IL-33 binding molecule or antigen-binding fragment thereof is a reduced IL-33 binding molecule or antigen-binding fragment thereof. In other words, the IL-33 binding molecule or antigen-binding fragment thereof inhibits or attenuates the activity of reduced IL-33. Suitably, the attenuation is by binding to reduced IL-33. Suitably, by binding to reduced IL-33 said binding molecule or antigen-binding fragment thereof also attenuates the activity of oxidised IL-33, suitably by preventing its conversion to the oxidised IL-33 form.

Suitably, the inhibition of the activity of oxidised IL-33 down-regulates or turns off RAGE dependent signalling and/or RAGE mediated effects.

Suitably, the IL-33 therapeutic agent has all of the inhibitory effects described above. Suitably, the reduced IL-33 therapeutic agent has all of the inhibitory effects described above.

Suitably the IL-33 therapeutic agent is a reduced IL-33 binding molecule or fragment thereof. Suitably the IL-33 therapeutic agent is a reduced IL-33 antibody or binding fragment thereof, suitably an anti-reduced IL33 antibody or binding fragment thereof.

Suitably, the therapeutic agent may inhibit or attenuate IL-33 signalling by binding to ST-2. Such therapeutic agents are referred to herein as “ST-2 inhibitors”. The ST2 inhibitor may be any such inhibitor known in the art, for example GSK3772847 (described in WO2013/165894) and RG6149 (WO2013/173761), both incorporated herein by reference. An ST-2 inhibitor can be used in combination with a second therapeutic agent that inhibits or attenuates RAGE signalling. The use of different therapeutic agents for inhibiting ST-2 signalling and RAGE-signalling may be advantageous for delivering different doses for inhibiting both pathways where the underlying pathology requires.

Formulations The therapeutic agents in the medical uses and methods described herein may be administered to a patient in the form of a pharmaceutical composition.

Suitably, any references herein to ‘therapeutic agent’ may also refer to a pharmaceutical composition comprising e.g. chemical inhibitor or antibody or antigen-binding fragment thereof that attenuates or inhibits activity of redIL-33 and/or oxIL-33. Suitably the pharmaceutical composition may comprise one or more therapeutic agents.

Suitably the therapeutic agents may be administered in a pharmaceutically effective amount for the in vivo treatment of kidney injury suitably diabetic kidney disease, in the medical use and method of treatment aspects herein.

Suitably a ‘pharmaceutically effective amount’ or ‘therapeutically effective amount’ of said one or more therapeutic agent(s) shall be held to mean an amount sufficient to achieve effective inhibition of redIL-33 and oxIL-33 activity and to achieve a benefit, e.g. to ameliorate symptoms of a disease or condition as recited in the medical uses/methods herein.

Suitably, the one or more therapeutic agent(s) or a pharmaceutical composition thereof may be administered to a human or other animal in accordance with the aforementioned methods of treatment/medical uses in an amount sufficient to produce a therapeutic effect.

Suitably, the one or more therapeutic agent(s) or a pharmaceutical composition thereof can be administered to such human or other animal in a conventional dosage form prepared by combining the one or more therapeutic agent(s) with a conventional pharmaceutically acceptable carrier or diluent according to known techniques.

It will be recognized by one of skill in the art that the form and character of the pharmaceutically acceptable carrier or diluent is dictated by the amount of the active ingredient(s) with which it is to be combined, the route of administration and other well-known variables.

The amount of one or more therapeutic agent(s) that may be combined with the carrier materials to produce a single dosage form will vary depending upon the subject treated and the particular mode of administration. Suitably, the pharmaceutical composition may be administered as a single dose, multiple doses or over an established period of time in an infusion. Suitably, dosage regimens also may be adjusted to provide the optimum desired response (e.g., a therapeutic or prophylactic response).

Suitably, the one or more therapeutic agent(s) will be formulated so as to facilitate administration and promote stability of the one or more therapeutic agent(s).

Suitably, pharmaceutical compositions are formulated to comprise a pharmaceutically acceptable, non-toxic, sterile carrier such as physiological saline, non-toxic buffers, preservatives and the like. Suitably the pharmaceutical composition may include sterile aqueous or non-aqueous solutions, suspensions, and emulsions. Suitable formulations for use in the therapeutic methods disclosed herein are described in Remington's Pharmaceutical Sciences (Mack Publishing Co.) 16th ed. (1980).

Suitably, pharmaceutical compositions for injectable use may include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will be preserved against the contaminating action of microorganisms, such as bacteria and fungi. Suitably, the carrier can be a solvent or dispersion medium containing, for example, water, ethanol, polyol (e.g. glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitably, prevention of the action of microorganisms can be achieved by various antibacterial and antifungal agents, for example, parabens, chlorobutanol, phenol, ascorbic acid, thimerosal and the like. In many cases, it will be suitable to include isotonic agents, for example, sugars, polyalcohols, such as mannitol, sorbitol, or sodium chloride in the pharmaceutical composition. Prolonged absorption of the injectable compositions can be brought about by including in the composition an agent which delays absorption, for example, aluminum monostearate and gelatin.

Suitably, sterile injectable solutions can be prepared by incorporating an active compound (e.g., one or more therapeutic agent(s) defined herein, by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation may be vacuum drying and freeze-drying, which yields a powder of an active ingredient plus any additional desired ingredient from a previously sterile-filtered solution thereof.

Methods of administering the one or more therapeutic agent(s) or a pharmaceutical composition thereof to a subject in need thereof are well known to or are readily determined by those skilled in the art.

Suitably, the route of administration of the one or more therapeutic agent(s) or pharmaceutical composition thereof may be, for example, oral, parenteral, by inhalation or topical. Suitably, the term parenteral as used herein includes, e.g., intravenous, intraarterial, intraperitoneal, intramuscular, subcutaneous, rectal, or vaginal administration.

Suitably, the one or more therapeutic agent(s) or pharmaceutical composition thereof may be orally administered in an acceptable dosage form including, e.g., capsules, tablets, aqueous suspensions or solutions.

Suitably, the one or more therapeutic agent(s) or pharmaceutical composition thereof may be administered by nasal aerosol or inhalation. Such compositions may be prepared as solutions in saline, employing benzyl alcohol or other suitable preservatives, absorption promoters to enhance bioavailability, and/or other conventional solubilizing or dispersing agents.

Suitably, parenteral formulations may be a single bolus dose, an infusion or a loading bolus dose followed with a maintenance dose. These compositions may be administered at specific fixed or variable intervals, e.g., once a day, or on an “as needed” basis.

Suitably, the one or more therapeutic agent(s) or pharmaceutical compositions thereof are delivered directly to the site of the disease or condition, for example the kidney, thereby increasing the exposure of the diseased tissue to the therapeutic agent. Suitably, the one or more therapeutic agent(s) or pharmaceutical compositions thereof are administered directly to the site of the disease or condition. Suitably, therefore, the one or more therapeutic agent(s) or pharmaceutical compositions thereof are administered to the site of kidney injury.

Suitably, therefore, the one or more therapeutic agent(s) or pharmaceutical composition thereof is formulated as a liquid composition.

Suitably, the components as recited hereinabove for preparing a pharmaceutical composition described herein may be packaged and sold in the form of a kit. Such a kit will suitably have labels or package inserts indicating that the associated pharmaceutical compositions are useful for treating a subject suffering from, or predisposed to a disease or disorder.

Suitably, the components for liquid formulations are processed, filled into containers such as ampoules, bags, bottles, syringes or vials, and sealed under aseptic conditions according to methods known in the art. Suitably the containers may be pressurised, suitably they may be aerosol containers. These containers may be included in a kit as described above. Suitably the kit may further comprise an inhaler device. Suitably the inhaler device comprises one or more therapeutic agent(s) or pharmaceutical composition described herein or is operable to comprise a container as described above which may comprise one or more therapeutic agent(s) or pharmaceutical composition described herein.

General

The practice of the present invention will employ, unless otherwise indicated, conventional methods of chemistry, biochemistry, molecular biology, immunology and pharmacology, within the skill of the art. Such techniques are explained fully in the literature. See, e.g., references (Gennaro (2000) Remington: The Science and Practice of Pharmacy. 20th edition, ISBN: 0683306472; Molecular Biology Techniques: An Intensive Laboratory Course, (Ream et al., eds., 1998, Academic Press; Methods In Enzymology (S. Colowick and N. Kaplan, eds., Academic Press, Inc.); Handbook of Experimental Immunology, Vols. I-IV (D.M. Weir and C. C. Blackwell, eds, 1986, Blackwell Scientific Publications); Sambrook et al. (2001) Molecular Cloning: A Laboratory Manual, 3rd edition (Cold Spring Harbor Laboratory Press); Handbook of Surface and Colloidal Chemistry (Birdi, K. S. ed., CRC Press, 1997); Ausubel et al. (eds) (2002) Short protocols in molecular biology, 5th edition (Current Protocols); PCR (Introduction to Biotechniques Series), 2nd ed. (Newton & Graham eds., 1997, Springer Verlag)).

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides or amino acids are the same in comparing the two sequences. This alignment and the percent homology or sequence identity can be determined using software programs known in the art, for example those described in section 7.7.18 Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489.

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### EMBODIMENTS

Embodiment 1 is for a method of treating kidney injury, the method comprising administering a therapy which inhibits both ST2 signalling and RAGE signaling, wherein said therapy comprises one or more therapeutic agents, such as 1 or 2 therapeutic agents.

Embodiment 2 is for a method according to embodiment 1, which attenuates or inhibits activity of reduced IL-33 protein (redIL-33) and thereby inhibits ST2 signalling.

Embodiment 3 is for a method according to embodiments 1 or 2, which attenuates or inhibits activity of oxidised IL-33 protein (oxIL-33) and thereby inhibits RAGE signaling.

Embodiment 4 is for a method according to any one of embodiments 1 to 3, wherein the kidney injury compnses inflammation.

Embodiment 5 is for a method according to embodiment 4, wherein the kidney injury is inflammatory.

Embodiment 6 is for a method according to embodiment 4 or 5, wherein the kidney injury is selected from diabetic kidney disease, fibrosis, glomerulonephritis (for example non-proliferative (such as minimal change glomerulonephritis, membrane glomerulonephritis, focal segmental glomerulosclerosis) or prolative (such as IgA nephropathy, membranoproliferative glomerulonephritis, post infectious glomerulonephritis, and rapidly progressive glomerulonephritis [such as Goodpastures syndrome and vasculitic disorders {which includes Wegners granulomatosis and microscopic polyangiitis}]), systemic lupus erythematosus, albuminuria, unilateral ureteral obstruction, Alport syndrome, polycystic kidney disease (PCKD), hypertensive glomerulosclerosis, chronic glomerulosclerosis, chronic obstructive uropathy, chronic tubulo-interstitial nephritis and ischemic nephropathy.

Embodiment 7 is for a method according to any one of embodiments 1 to 6, wherein the kidney injury is diabetic kidney disease.

Embodiment 8 is for a method according to any one of embodiments 1 to 7, wherein the therapeutic agent or agents is/are independently selected from a chemical inhibitor and an antibody or antigen-binding fragment thereof.

Embodiment 9 is for a method according to embodiment 8, wherein the therapeutic agent or agents comprise an antibody or antigen-binding fragment thereof.

Embodiment 10 is for a method according to embodiment 8 or 9, wherein the antibody or antigen-binding fragment thereof binds specifically to IL-33.

Embodiment 11 is for a method according to embodiment 10, wherein the antibody or antigen-binding fragment has the complementarity determining regions (CDRs) of a variable heavy domain (VH) and a variable light domain (VL) pair selected from Table 1.

Embodiment 12 is for a method according to embodiment 10 or 11, wherein the antibody or antigen-binding fragment thereof specifically binds to redIL-33 and attenuates or inhibits activity of redIL-33, thereby inhibiting ST2 signalling.

Embodiment 13 is for a method according to any of embodiments 10 to 12, wherein the antibody or antigen-binding fragment thereof prevents binding of oxidised IL-33 to RAGE, thereby inhibiting RAGE-EGFR signalling.

Embodiment 14 is for a method according to any one of embodiments 10 to 13, wherein the antibody or an antigen-binding fragment thereof binds to redIL-33 with a binding affinity of less than or equal to 100 pM, or less than or equal to 10 pM, for example less than or equal to 1 pM, such as 0.5 pM, in particular 0.05 pM (for example when measured using KinExA).

Embodiment 15 is for a method according to any one of embodiments 10 to 14, wherein the antibody or an antigen-binding fragment thereof binds to redIL-33 with a k(on) greater than or equal to 10⁵M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹, in particular greater than or equal to 10⁷M⁻¹sec⁻¹.

Embodiment 16 is for a method according to any one of embodiments 10 to 15, wherein the antibody or an antigen-binding fragment thereof binds to redIL-33 with a k(off) less than or equal to 5×10⁻¹ sec⁻¹, 10⁻¹ sec⁻¹, 5×10⁻² sec⁻¹, 10⁻² sec⁻¹, 5×10⁻³ sec⁻¹ or 10⁻³ sec⁻¹, in particular less than or equal to 10⁻³ sec⁻¹.

Embodiment 17 is for a method according to any one of embodiments 10 to 16, wherein the antibody or an antigen-binding fragment attenuates or inhibits the activity of oxIL-33 and thereby inhibits RAGE signaling.

Embodiment 18 is for a method according to any one of embodiments 9 to 17, wherein the antibody or antigen-binding fragment comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

Embodiment 19 is for a method according to any one of embodiments 9 to 18, wherein the antibody or antigen-binding VH and VL of said antibody or antigen-binding fragment thereof comprise amino acid sequences at least 85%, 90%, 95%, 96%, 97%, 98% or 99% identical to SEQ ID NO: 1 and SEQ ID NO: 19, respectively.

Embodiment 20 is for a method according to embodiment 19, wherein the antibody or antigen-binding fragment comprises a VH having the sequence of SEQ ID NO: 1 and a VL having the sequence of SEQ ID NO:19.

Embodiment 21 is for a method of any one of embodiments 8 to 20, wherein the antibody or an antigen-binding fragment thereof is a human antibody, a chimeric antibody, and a humanized antibody.

Embodiment 22 is for a method of any one of embodiments 8 to 21, wherein the antibody or the antibody or an antigen-binding fragment thereof is a naturally-occurring antibody, an scFv fragment, an Fab fragment, an F(ab′)2 fragment, a minibody, a diabody, a triabody, a tetrabody, or a single chain antibody.

Embodiment 23 is for a method of any one of embodiments 8 to 22, wherein the antibody or an antigen-binding fragment thereof is a monoclonal antibody.

Embodiment 24 is for a method according to any one of embodiments 1 to 23, wherein at least two therapeutic agents are employed, for example wherein one therapeutic agent inhibits ST2 signalling and the second therapeutic agent inhibits RAGE signaling, for example wherein the therapeutic agent prevents binding of the ligand or ligands to the receptor or receptors.

Embodiment 25 is for a method according to any one of embodiments 1 to 24, wherein one therapeutic agent is employed, wherein the therapeutic agent inhibits both ST2 and RAGE signaling, for example wherein the agent prevents binding of the ligand to both receptors.

Embodiment 26 is for a method of any preceding embodiment, wherein the RAGE signalling is RAGE-EGFR-signalling.

Embodiment 27 is for a method of embodiment 26, wherein inhibition of RAGE-EGFR signaling down-regulates or inhibits RAGE-EGFR mediated effects.

Embodiment 28 is for a method of embodiment 27, wherein the RAGE-EGFR mediated effect is abnormal epithelium remodelling.

Embodiment 29 is for a therapeutic agent that inhibits or attenuates the activity of reduced IL-33 to thereby inhibit or attenuate ST2 signaling, for use in the treatment of kidney injury, or for use in the manufacture of a medicament for the treatment of kidney injury, wherein the treatment further comprises inhibiting or attenuating the activity of oxidized IL-33 to thereby inhibit or attenuate RAGE signaling.

Embodiment 30 is for a therapeutic agent that inhibits or attenuates the activity of oxidized IL-33 to thereby inhibit or attenuate RAGE signaling, for use in the treatment of kidney injury or for use in the manufacture of a medicament for the treatment of kidney injury, wherein the treatment further comprises inhibiting or attenuating the activity of reduced IL-33 to thereby inhibit or attenuate ST2 signaling.

Embodiment 31 is for a therapeutic agent that inhibits or attenuates the activity of reduced IL-33 to thereby inhibit or attenuate ST2 signaling, and a therapeutic agent that inhibits or attenuates the activity of oxidized IL-33 to thereby inhibit or attenuate RAGE signaling, for use in the treatment of kidney injury, or for use in the manufacture of a medicament for the treatment of kidney injury.

Embodiment 32 is for a therapeutic agent that inhibits or attenuates the activity of reduced IL-33 and oxidized IL-33 to thereby inhibit or attenuate ST2 signaling and RAGE signaling, for use in the treatment of kidney injury, or for use in the manufacture of a medicament for the treatment of kidney injury.

Embodiment 33 is for a therapeutic agent for use, or use, according to any one of embodiments 29 to 32, which is an antibody or antigen binding fragment thereof.

Embodiment 34 is for a therapeutic agent for use according to embodiment 33, wherein the antibody or antigen-binding fragment thereof is as characterized in any of embodiments 10 to 23.

Embodiment 35 is for a therapeutic agent for use according to any one of embodiments 29 to 34, wherein the kidney injury is as characterized in any of embodiments 4 to 7.

Embodiment 36 is for a therapeutic agent for use according to any of embodiments 29 to 35, wherein the RAGE signalling is RAGE-EGFR-signalling.

Embodiment 37 is for a therapeutic agent for use according to any of embodiments 29 to 36, wherein the inhibition or attenuation of RAGE-EGFR signaling down-regulates or inhibits RAGE-EGFR mediated effects.

Embodiment 38 is for a therapeutic agent for use according to any of embodiments 37, wherein the RAGE-EGFR mediated effect is abnormal epithelium remodelling.

Embodiment 39 is for a method according to any one of embodiments 9 to 26, or the therapeutic agent for use according to any one of embodiments 31 to 38, wherein the antibody or antigen-binding fragment thereof competes for binding to redIL-33 with 33_640087-7B, for example as determined by Homogeneous Time-Resolved Fluorescence.

### EXAMPLES

### Example 1

### Assessing the Role of IL-33 Biology in Human Diabetic Kidney Disease

A range of inflammatory mediators have been shown to be upregulated in the context of kidney disease (Thomas et al., Nat reviews Dis Primers., 2015). The methods described below were used to determine how expression levels of interleukin-33 (IL-33) rank among other inflammatory mediators known to be involved with kidney disease.

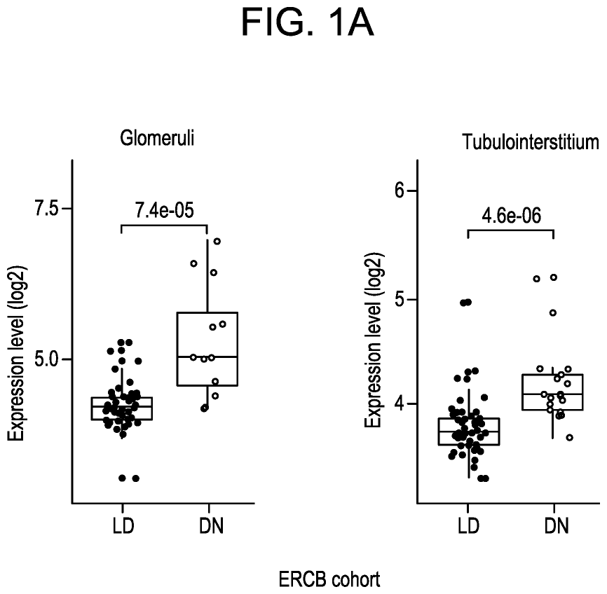

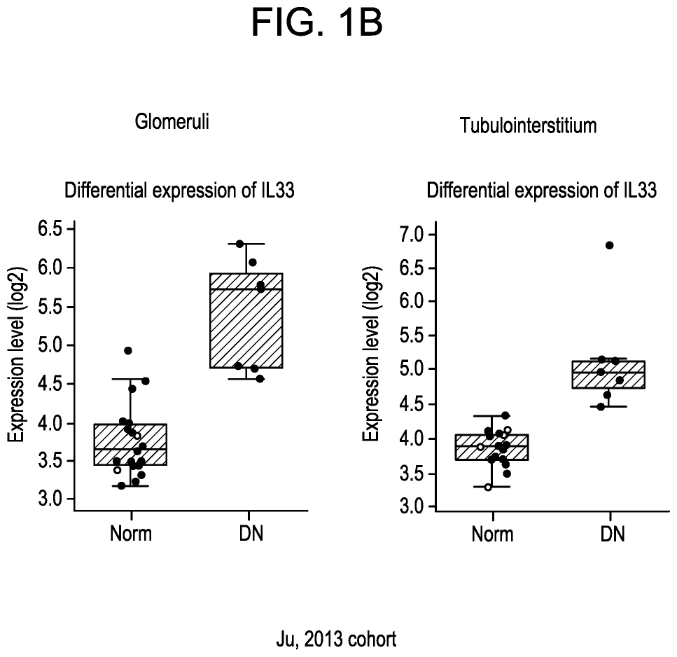

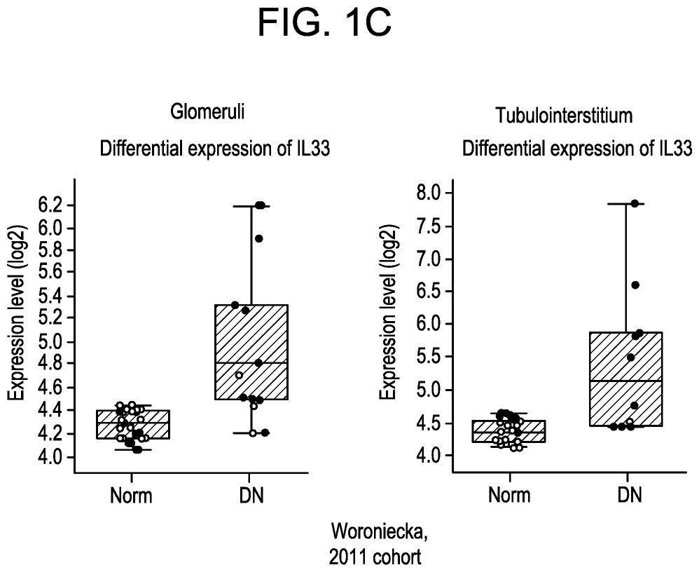

Published RNA transcriptome material from three different cohorts of patients with diabetic nephropathy (FIG. 1: European Renal cDNA Bank cohort (FIG. 1A), Hu 2013 cohort (FIG. 1B), and Woroniecka, 2013 cohort (FIG. 1C)) was analysed. RNA expression levels were measured within glomeruli and tubulointerstitium tissue samples. Transcriptome analysis was performed based on RNA sequence count reads according to standardised methodology (Ju et al 2013; Woroniecka et al, 2013).

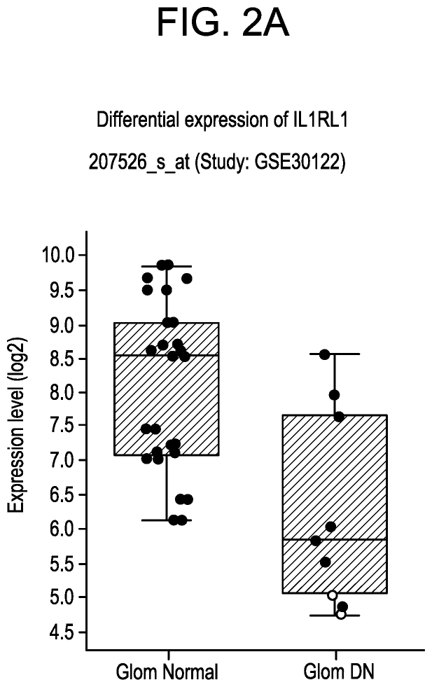

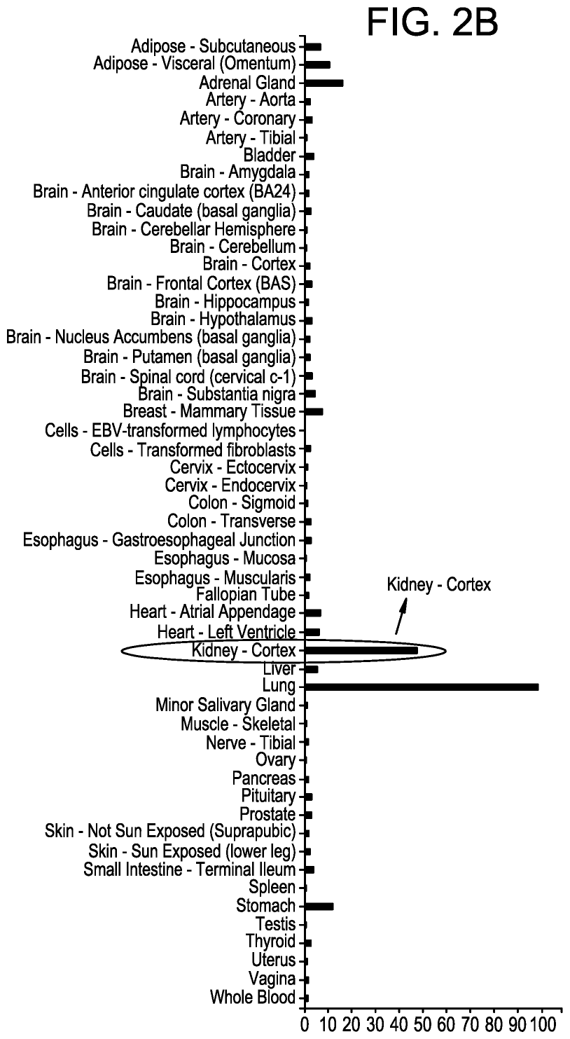

It was found IL-33 ranked among the highest overexpressed cytokines in the kidneys of subjects with diabetic nephropathy (DN) from all three cohorts. IL-33 expression was upregulated in DN kidney in both glomeruli and tubulointerstitium (FIG. 1). Surprisingly, it was found that the expression of the IL-33 cognate receptor ST2 (IL1RL1), was down regulated in the glomeruli of at least one cohort of diabetic nephropathy patients (FIG. 2B). It has previously been shown that ST2 is expressed at comparatively high levels in the kidney, primarily the renal cortex, to other tissues in the body (FIG. 2A). This suggests some sort of compensatory mechanism may exist to downregulate ST2 expression to prevent overactivity resulting from elevated levels of IL-33.

### Example 2

### Assessing the Level of IL-33 in Kidneys in Pre-Clinical Models of Kidney Disease

The methods described below were used to assess whether the level of IL-33 expression is elevated in a range of pre-clinical models of kidney disease. IL-33 mRNA expression levels were quantified in kidney samples derived from a Type II diabetic nephropathy (T2DN) mouse model (db/db mice obtained from Jackson laboratory, catalogue number 000697, which have had uninephrectomy (unx)), a hypertensive nephropathy (HN) mouse model (5/6 Npx mice obtained by surgery intervention removing 5/6 nephron mass in BL6 mice (see Wang et al J. Vis. Exp., 129; e55825; 2017), an obstructive nephropathy mouse model (ON) (obtained from surgical unilateral ureteral obstruction in BL6 mice (see Hesketh et al J. Vis. Exp., 94; e52559; 2014)) and a mouse model of Type 1 diabetic nephropathy (T1DN) (using chemical ablation with STZ, see Chow et al 69; 73-80; Kidney International, 2006).

Disease modelling was carried out following well-established protocols (see, for example, Wang et al J. Vis. Exp., 129; e55825; 2017, Hesketh et al J. Vis. Exp., 94; e52559; 2014, Chow et al 69; 73-80; Kidney International, 2006, Zhou et al Am J Trans! Res 8: 1339-54, 2016; Yang et al Drug Discov Today Dis Models, 7; 13-19; 2010).

For T2DN mice, the relative IL-33 kidney expression levels were assayed in db/db mice at 8 weeks of age or at 16 weeks in db/db mice with uninephrectomy (db/db+unx). For T1DN mice, the relative IL-33 kidney expression levels were assayed in mice with STZ and without STZ at 12 weeks. For HN mice, the relative IL-33 kidney expression levels were assayed in mice following 5/6 NPX and sham mice at 6 weeks. For ON mice, relative IL-33 kidney expression levels were assayed in UUO mice and in sham control at 7 days. Samples were prepared for transcriptome analysis according to Zhou et al Am J Transl Res 8: 1339-54, 2016. Relative levels of expression were normalised to the relative expression level of GAPDH in each cohort.

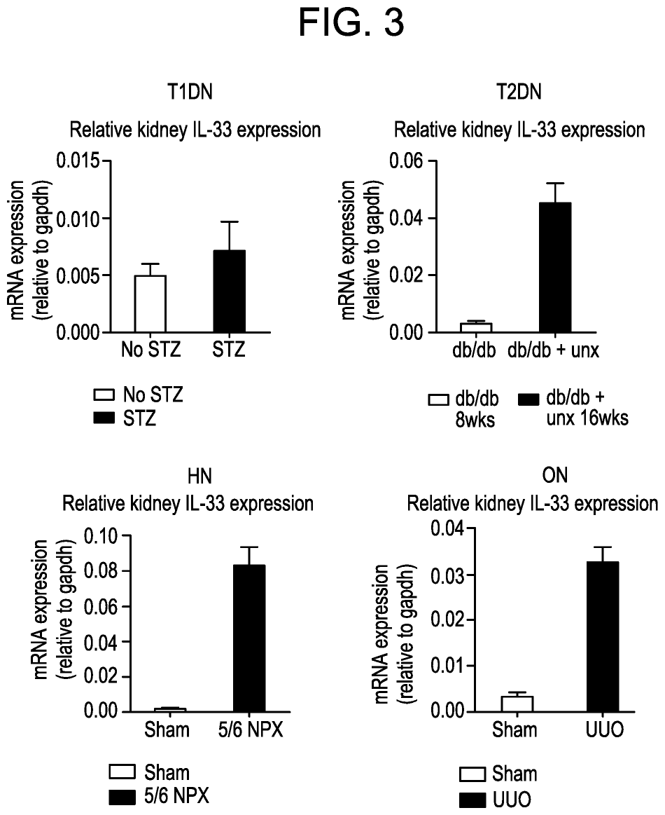

As shown in FIG. 3, normalised IL-33 expression is elevated in (a) T2DN db/db unx mice, (b) T1DN mice with STZ, (c) HN mice with 5/6 NPX and (d) ON mice with UUO. Expression levels were normalised to GAPDH in each cohort. These results show that IL-33 expression levels are increased in the kidney across all pre-clinical models of kidney disease in comparison to controls.

### Example 3

### RAGE is Upregulated in Models of Kidney Disease

The existence of two biologically active forms of IL-33, redIL-33 and oxIL-33, had been previously described. As reported in WO2016/156440, reduced IL-33 (redIL-33) is the isoform shown to signal via the ST2 signalling pathway. Conversion of red-IL33 to the oxidised form (oxIL-33) by disulfide bond formation between native cysteines is proposed as a switch-off mechanism for redIL-33 signalling. However, in WO2016/156440, the inventors characterised a novel signalling pathway through which oxIL-33 stimulates signalling via RAGE (see, for example, FIG. 58 in WO2016/156440).

Given the existence of the RAGE signalling pathway, the change in expression profile of RAGE in pre-clinical models of kidney disease was determined.

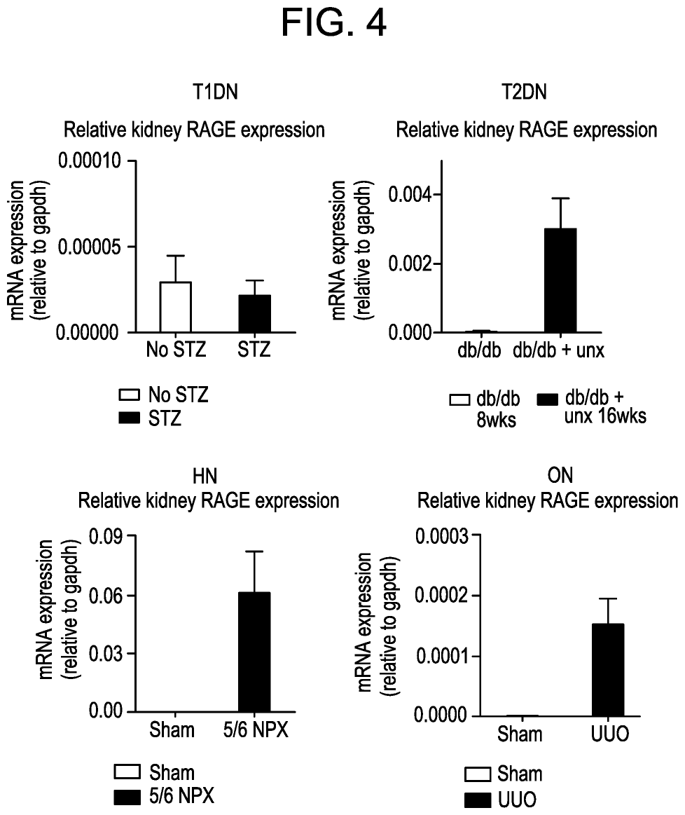

RAGE expression was quantified using the same models and methods described in Example 2. As shown in FIG. 4, normalised RAGE expression is elevated in (a) T2DN db/db unx mice, (b) T1DN mice with STZ, (c) HN mine with 5/6 NPX and (d) ON mice with UUO. Expression levels were normalised to GAPDH in each cohort. These results show that RAGE expression is upregulated in all tested pre-clinical models of kidney disease.

### Example 4

### RAGE and ST2 Signalling Contribute to Kidney Dysfunction In Vivo

As an increase in both RAGE and IL-33 expression levels was observed in preclinical models of CKD, the contribution of signalling pathways involving these molecules toward the pathology of kidney disease was determined.

Briefly, ST2 and RAGE-mediated cytotoxic effects were investigated in the db/db uninephrectomy mouse model described above. ST2 and RAGE-dependent signalling were blocked in separate mice. Kidney damage was assessed by monitoring the concentration of albumin in the urine (albuminuria).

Albuminuria was measured as the amount of albumin in the urine normalised to the concentration of creatinine, to compensate for variations in the urine concentration. This value is called the albumin: creatinine ratio (UACR).

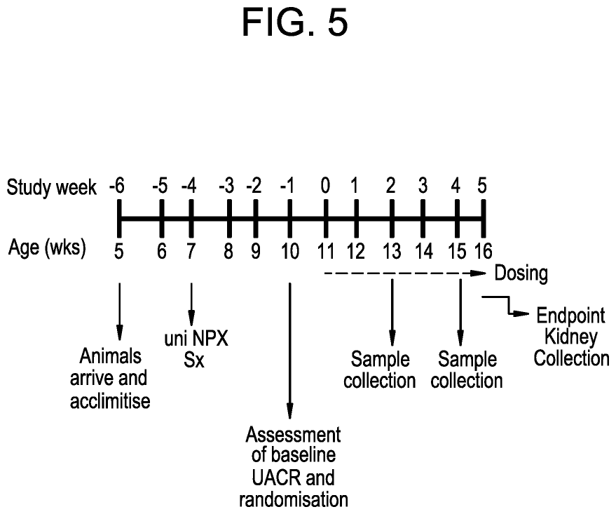

The study was carried out as shown in FIG. 5. Briefly, mice were uninephrectomised at 7 weeks to accelerate kidney damage. Mice were dosed with either 10 mg/Kg 3×/week anti-RAGE hu-IgG1, anti-ST2 muIgG1 or huNUP228 IgG1, which served as a negative control. Urine samples were collected on days 10, 13 and 15 and assayed for albumin and creatinine levels using Cobas® immunochemistry. Glomerular damage was also assayed by internal pathology scoring using mesangial expansion, scarcity of nuclei and decrease capillary luminal spaces.

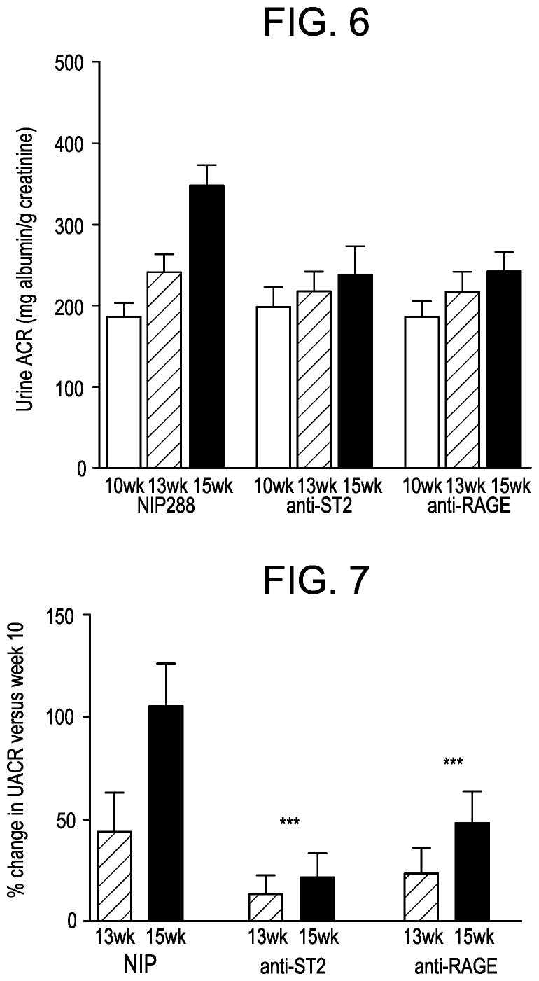

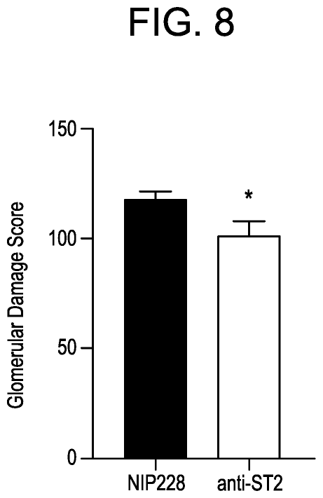

The results show that blocking ST2 and RAGE signalling both led to a significant reduction in UACR score (FIGS. 6 and 7). FIG. 8 also demonstrates a significant reduction in glomerular damage was achieved by blocking ST2 signalling in comparison to the negative control (* indicates significantly less (P<0.05) GDS than isotype controls).

### Example 5

### Oxidised IL-33 Drives Formation of a Signaling Complex Between RAGE and EGFR

In Cohen, E. S. et al. Nat. Commun. 6:8327 (2015), the discovery of an oxidized, disulphide bonded form of IL-33 (oxIL-33) is described. OxIL-33 was shown not to bind ST2 or activate ST2-dependent signalling. Subsequently (see WO2016156440A1), it was shown that oxIL-33 binds the Receptor for Advanced Glycation End products (RAGE) and signals in a RAGE-dependent manner to activate STATS and affect epithelial migration.

To further explore the function of oxIL-33, epithelial cells were stimulated with IL-33 in reduced or oxidised forms (redIL-33 and oxIL-33) and signaling pathways were investigated. Here it is shown that oxIL-33 is a novel ligand for a complex of the receptor for advanced glycation end products (RAGE) and the epidermal growth factor receptor (EGFR), leading to profound effects on epithelial function.
1. Cloning and Expression of Human Mature and Cysteine-Mutated Variants of IL33

cDNA molecules encoding the mature component of human IL-33 (112-270); accession number (UniProt) 095760 (also referred to as IL33-01 or IL-33), and a variant with the 4 cysteine residues mutated to serine (also referred to as IL33-16 or IL-33[C->S]) were synthesized by primer extension PCR and cloned into pJexpress 411 (DNA 2.0). The wild type (WT) and mutant IL-33 coding sequences were modified to contain a 10xHis, Avitag, and Factor-Xa protease cleavage site (MHHHHHHHHHHAAGLNDIFEAQKIEWHEAAIEGR SEQ ID NO:43) at the N-terminus of the proteins. N-terminal tagged His10/Avitag IL33-01 (WT, SEQ ID NO:44) and N-terminal tagged His10/Avitag IL33-16 (WT, SEQ ID NO:45) were generated by transforming E. coli BL21(DE3) cells. Transformed cells were cultured in autoinduction media (Overnight Express™ Autoinduction System 1, Merck Millipore, 71300-4) at 37° C. for 18 hours before cells were harvested by centrifugation and stored at −20° C. Cells were resuspended in 2×DPBS containing complete EDTA-free protease inhibitor cocktail tablets (Roche, 11697498001) and 50 U/ml Benzonase nuclease (Merck Millipore, 70746-3) and lysed by sonication. The cell lysate was clarified by centrifugation at 50,000 ×g for 30 min at 4° C. IL-33 proteins were purified from the supernatant by immobilized metal affinity chromatography, loading on a HisTrap excel column (GE Healthcare, 17371205) equilibrated in 2×DPBS, 1 mM DTT at 5 ml/min. The column was washed with 2×DPBS, 1 mM DTT, 20 mM Imidazole, pH 7.4 to remove impurities and then 2×DPBS, 0.1% Triton X-114 to deplete the immobilised protein of endotoxin. Following further washing with 2×DPBS, 1 mM DTT, 20 mM Imidazole, pH 7.4, the sample was eluted with 2×DPBS, 1 mM DTT, 400 mM Imidazole, pH 7.4. IL-33 was further purified by size exclusion chromatography using a HiLoad Superdex 75 26/600 pg column (GE Healthcare, 28989334) in 2×DPBS at 2.5 ml/min. Peak fractions were analysed by SDS PAGE. Fractions containing pure IL-33 were pooled and the concentration determined by absorbance at 280 nm. Final samples were analysed by SDS-PAGE.

To generate untagged IL-33, N-terminal tagged His10/Avitag IL33 was incubated with 10 units of Factor Xa (GE healthcare, 27084901) per mg of protein in 2×DPBS buffer at RT for 1 hour. Untagged IL-33 was purified using SEC chromatography in 2×DPBS on a HiLoad 16/600 Superdex 75 pg column (GE healthcare, 28989333) with a flow rate of 1 ml/min.
2. Generation and Purification of Oxidised IL-33 (oxIL-33)

Reduced IL33 was oxidised by dilution to a final concentration of 0.5 mg/ml in 60% IMDM medium (with no phenol red), 40% DPBS and incubation at 37° C. for 18 hours. Aggregates generated during the oxidation process were removed from the sample by loading it on a HiTrap Capto Q ImpRes anion exchange column (GE Healthcare, 17547055). Prior to loading, the sample was modified by the addition of 1 M Tris, pH 9.0 until the pH reached 8.3 and the addition of 5 M NaCl to a final concentration of 125 mM—under these loading conditions, aggregates bound to the column and monomeric oxIL-33 flowed through without binding and was collected. Tags were cleaved from the oxIL-33 by incubation with Factor Xa (NEB, P8010L) at a final concentration of 1 μg Factor Xa per 50 μg of oxIL-33 for 120 min at 22° C. To deplete the sample of any remaining reduced IL-33, soluble human ST2S extracellular domain fused to human IgG1 Fc-His6 was incubated with the sample for 30 min at 22° C. and bound the reduced IL-33. The sample was concentrated in a centrifugal concentrator with a 3,000 Da cut-off and loaded on a HiLoad Superdex 75 26/600 pg column (GE Healthcare, 28989334) at a flow rate of 2 ml/min, which separated the monomeric oxIL-33 from the other sample components. Fractions containing pure oxIL-33 were pooled and concentrated and the final concentration of the sample was determined via UV absorbance spectroscopy at 280 nm. Final product quality was assessed by SDS-PAGE, HP-SEC and RP-HPLC.
3. Cloning, Expression and Purification of Human ST2 ECD

A cDNA encoding the naturally occurring ST2S soluble isoform of ST2 (UniProt accession Q01638-2) without the endogenous signal peptide (amino acid residues 19-328) was amplified by PCR with primers encoding extensions compatible with Gibson assembly and a CD33 signal peptide fused to the N-terminus of the ST2S coding sequence. A coding sequence for human IgG1 Fc with a C-terminal His6-tag was similarly amplified. The ST2S cDNA and IgG1 Fc-His6 cDNA were assembled using Gibson assembly with pDEST12.2 OriP, a mammalian, CMV-promoter driven expression vector bearing the OriP origin of replication from EBV, allowing episomal maintenance in cell lines expressing the EBNA-1 protein. For protein expression, the plasmid was transiently transformed into a suspension culture of CHO cells overexpressing EBNA-1 using polyethyleneimine as the transfection reagent. Conditioned medium containing the secreted ST2S-Fc-His6 fusion protein was collected 7 days post-transfection and loaded on a HiTrap MabSelect SuRe (Protein A, GE Healthcare, 11-0034-95) affinity chromatography column at 2 ml/min. The column was washed with 2×DPBS and the protein eluted with 25 mM Sodium acetate, pH 3.6. Fractions containing ST2S-Fc-His6 were pooled and loaded on a HiLoad Superdex 200 26/600 pg column (GE Healthcare, 28989336) equilibrated in 2×DPBS at 2 ml/min. Fractions containing pure ST2S-Fc-His6 protein were pooled and the concentration determined by absorbance at 280 nm. Final samples were analysed by SDS-PAGE.
4. Cloning, Expression and Purification of Human Asialoglycoprotein Receptor (ASGPR) ECD

A cDNA encoding the extracellular domain (ECD) of the Asialoglycoprotein receptor (UniProt accession P07306) without the cytoplasmic and transmembrane domains (amino acid residues 62-291) was chemically synthesized at Geneart with a CD33 signal peptide followed by an His10 Avi Tag sequence fused to the N-terminus of the ECD domain. The construct was cloned directly into pDEST12.2 OriP, a mammalian, CMV-promoter driven expression vector bearing the OriP origin of replication from EBV, allowing episomal maintenance in cell lines expressing the EBNA-1 protein. For protein expression, the plasmid was transiently transformed into a suspension culture of HEK Freestyle 293F cells using 293 Fectin as the transfection reagent. Conditioned medium containing the secreted HisAVi hASGPR ECD fusion protein was collected 7 days post-transfection by immobilized metal affinity chromatography, loading on a HisTrap excel column (GE Healthcare, 17371205) equilibrated in 2×DPBS, at 4 ml/min. The column was washed with 2×DPBS, 40 mM Imidazole, pH 7.4 to remove impurities and the sample was eluted with 2×DPBS, 400 mM Imidazole, pH 7.4. Human ASGPR ECD was further purified by size exclusion chromatography using a HiLoad Superdex 75 16/600 pg column (GE Healthcare, 28-9893-33) in 2×DPBS at 1 ml/min. Peak fractions were analysed by SDS PAGE. Fractions containing pure monomeric ASGPR were pooled and the concentration determined by absorbance at 280 nm. Final samples were analysed by SDS-PAGE.
5. The Oxidised Form of IL-33 Activates MAP Kinase Pathways

Normal Human Bronchial Epithelial (NHBE) cells (CC-2540) were obtained from Lonza and were maintained in complete BEGM media (Lonza) according to the manufacturer's protocol. NHBEs were harvested with accutase (PAA, #L1 1-007) and seeded at 1×10⁶/2 ml in a 6-well dish (Corning Costar, 3516) in culture media [BEGM (Lonza CC-3171) and supplement kit (Lonza CC-4175)]. Cells were incubated at 37° C., 5% CO₂ for 18-24 hours. After this time, media was aspirated, and the cells were washed twice with 1 ml PBS before the addition of starve media (BEGM (Lonza CC-3171) supplemented with 1% Penicillin/Streptomycin). The plates were then incubated at 37° C., 5% CO₂ for a further 18-24 hours before stimulation.

MAP kinase phosphorylation antibody array kits (ab211061) were purchased from Abcam and experiments were carried out as per the manufacturer's instructions. NHBEs in a 6 well dish that had been starved for 18-24 h were left untreated or treated with 30 ng/ml of either reduced IL-33, IL-33-16 or oxidised IL-33 before being returned to an incubator 37° C., 5% CO₂ for 10 mins (see Table 2 for activators used in this assay). The plates were removed from the incubator and the cells washed with ice-cold PBS before the addition of 100 μl/per well of 1× lysis buffer supplied with the kits. Protein extracts were transferred to 1.5 ml tubes before being clarified at 14,000 rpm at 4° C. Protein concentration was determined using the BCA technique (Thermo, 23225) and 250 μof total protein was used per array membrane. All subsequent steps were carried out following the manufacturer's instructions. Membranes were visualised on a LiCor C-digit and quantified using Image Lite studio.

TABLE 2

| | | | Final |
|---|---|---|---|
| | | Reconstitute | conc |
| Agonist | Identifier | in | (μg/ml) |
| Untagged oxidised IL33-01 | RD15 | PBS | 100 |
| Untagged IL33-01 | Jul. 24, 2015 | PBS | 100 |
| Untagged IL33-16 | Nov. 12, 2015 | PBS | 100 |
| EGF | 236-EG-200 | PBS | 100 |

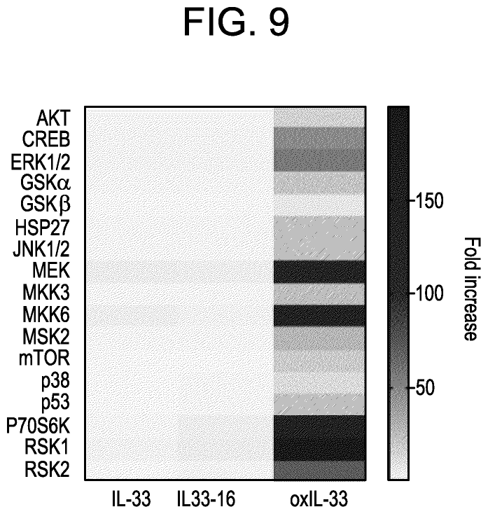

In contrast to the wild type (IL-33) and C->S (IL-33 [C->S]) reduced forms of IL-33 (IL33-01 and IL33-16, respectively), oxidised IL33 (oxIL-33) activated multiple key signalling molecules (FIG. 9) coinciding with pathways engaged by receptor tyrosine kinases (RTK).
6. The Oxidised Form of IL-33 Activates Epidermal Growth Factor Receptor (EGFR)

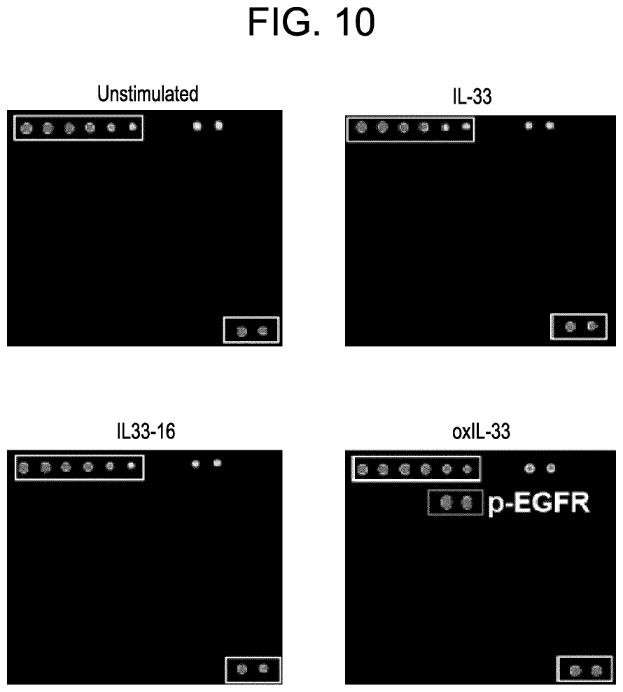

To try and identify receptor tyrosine kinases (RTK) that were activated by oxIL-33, screening was performed using a 71 RTK array. RTK phosphorylation antibody array kits (ab193662) were purchased from Abcam and experiments were carried out as per the manufacturer's instructions. NHBEs were cultured and seeded at 1×10⁶/₂ ml in a 6-well plate (Corning Costar, 3516) in culture media [BEGM (Lonza CC-3171) and supplement kit (Lonza CC-4175)]. Cells were incubated at 37° C., 5% CO₂ for 18-24 hours. After this time, media was aspirated, and the cells were washed twice with 1 ml PBS before the addition of starve media (BEGM (Lonza CC-3171) without supplement kit). The plates were then incubated at 37° C., 5% CO₂ for a further 18-24 hours before stimulation. Following the same steps previously described for the MAP kinase array, cells were activated (Table 2 activators), lysed and 250 μg of total protein was used per array membrane. All subsequent steps were carried out following the manufacturer's instructions. Membranes were visualised on a LiCor C-digit and quantified using Image Lite studio. There was no response detected to either reduced wild type (IL-33) or C->S (IL-33 [C->S]) IL-33 (IL33-01 and IL33-16, respectively). However, oxIL-33 (oxidised IL-33-01) triggered a positive signal on the RTK array corresponding to epidermal growth factor receptor (EGFR) (FIG. 10).

The ability of oxIL-33 (oxidised IL-33-01) to stimulate EGFR signalling was confirmed by additional methods. Upon activation, EGFR is phosphorylated at Tyr1068 and this phospho-EGFR can be detected using a homogeneous FRET (fluorescence resonance energy transfer) HTRF® (Homogeneous Time-Resolved Fluorescence, Cisbio International) assay (Cisbio kit #64EG1PEH). Briefly, NHBEs were plated at 5×10⁵/100 μl in a 96-well plate (Corning Costar, 3598) in culture media [BEGM (Lonza CC-3171) and supplement kit (Lonza CC-4175)]. The plates were incubated at 37° C., 5% CO₂ for 18-24 hours. After this time, media was aspirated, and the cells were washed twice with 0.2 ml PBS before the addition of starve media (BEGM (Lonza CC-3171) without supplement kit). The plates were then incubated at 37° C., 5% CO₂ for a further 18-24 hours before stimulating with increasing concentrations of IL-33-01, IL-33-16 and oxIL-33 (oxidised IL-33-01) and EGFR ligands (Tables 2 & 3) before being returned to an incubator 37° C., 5% CO₂ for 10 mins. The media was aspirated and replaced with 50 μl of lysis buffer per well (Cisbio, 64EG1PEH). The assay was then carried out as per the manufacturer's instructions (Cisbio, 64EG1PEH). Time resolved fluorescence was read at 620 nm and 665 nm emission wavelengths using an EnVision plate reader (Perkin Elmer). Data were analysed by calculating the 665/620 nm ratio and EC50 values determined using GraphPad Prism software by curve fitting using a four-parameter logistic equation.

TABLE 3

| | | | | Final |
|---|---|---|---|---|
| | | | Reconstituted | conc |
| Agonist | Supplier | Identifier | in | (μg/ml) |
| TGFα | R&D | 239-A-100 | 10 mM | 100 |
| | systems | | acetic acid | |
| HB-EGF | R&D | 259-HE-050/CF | PBS | 100 |
| | systems | | | |
| Amphiregulin | R&D | 262-AR-100/CF | PBS | 100 |
| (AREG) | systems | | | |
| Betacellulin/ | R&D | 261-CE-010/CF | PBS | 100 |
| BTC | systems | | | |
| Epiregulin | R&D | 1195-EP-025/CF | PBS | 100 |
| | systems | | | |
| Epigen | R&D | 6629-EP-025/CF | PBS | 100 |
| | systems | | | |
| HMGB1 | R&D | 1690-HMB-050 | PBS | 200 |
| | systems | | | |
| S100A8/A9 | R&D | 8226-S8-050 | PBS | 500 |
| | systems | | | |
| S100A12 | R&D | 1052-ER-050 | PBS | 200 |
| | systems | | | |
| S100B | R&D | 1820-SB-050 | PBS | 200 |
| | systems | | | |

Similarly, EGFR phosphorylation was assessed in the epithelial cell line A549 utilizing HTRF assay as previously mentioned in this section. Briefly, A549s were obtained from ATCC and cultured in RPMI GlutaMax medium supplemented with 1% Penicillin/Streptomycin and 10% FBS. Cells were harvested with accutase (PAA, #L1 1-007) and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Cells were stimulated with increasing concentrations of IL-33-01, IL-33-16 and oxIL-33, EGFR ligands and RAGE ligands (Tables 2 & 3) before being returned to an incubator 37° C., 5% CO₂ for 10 mins. The media was aspirated and replaced with 50 μl of lysis buffer per well (Cisbio, 64EG1PEH). The assay was then carried out as per the manufacturer's instructions (Cisbio, 64EG1PEH). Time resolved fluorescence was read at 620 nm and 665 nm emission wavelengths using an EnVision plate reader (Perkin Elmer). Data were analysed by calculating the 665/620 nm ratio and EC50 values determined using GraphPad Prism software by curve fitting using a four-parameter logistic equation.

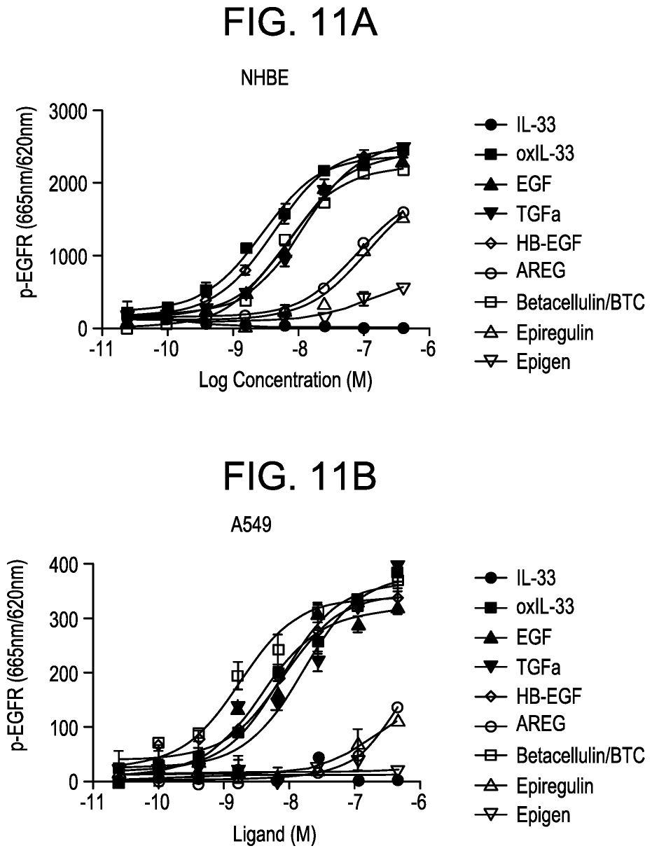

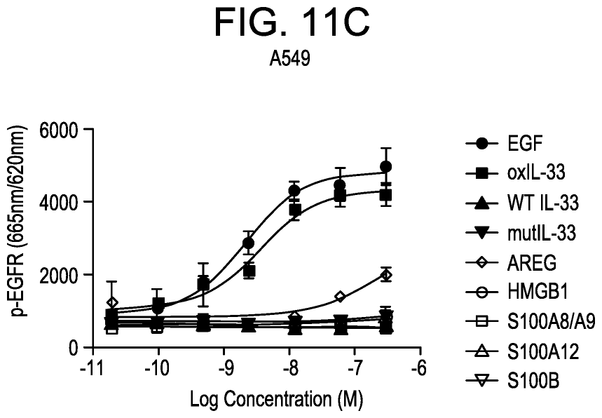

In both NHBE and A549 cells, oxIL-33 promoted phosphorylation of the EGFR similarly to a bona fide agonist, EGF (FIG. 11). This was not replicated by other RAGE ligands tested.
7. Western Blotting of Signaling Components

Western blot experiments were performed to further investigate which elements of the EGFR signalling complex are activated in response to oxIL-33. NHBEs were cultured and plated in 6 well dishes as described above in section 5. Following serum starvation, cells were stimulated with oxIL-33 (30 ng/ml) for between 5 to 240 minutes. The media was then aspirated and the cells were washed with ice-cold PBS before the addition of 150 μl of lysis buffer [1×LDS sample buffer (Thermo, NP0008), 10 mM MgC12 (VWR, 7786-30-3), 2.5% β-mercaptoethanol (Sigma, M6250) and 0.4 μg/mlbenzonase (Millipore, 70746)]. Cells were left on ice for 10 mins before lysate was transferred to 1.5 ml tubes and heated to 90° C. for 5 mins. Solutions were transferred to new 1.5 ml tubes and 10 μl of sample along with 5 μl of protein ladder (BioRad, 1610374) was run on a 4-12% SDS-PAGE gel (Thermo, NWO4127BOX) in MES running buffer (B0002). Gels were transferred onto PVDF membranes (BioRad, 1704156) using a Transblot Turbo (BioRad). PVDF membranes were blocked in PBS-tween solution containing 5% skimmed milk powder (Marvel) for 10 minutes. Membranes were then incubated with primary antibodies in PBS-tween containing 5% BSA over night at 4° C. The membranes were then washed five times with PBS-tween and then incubated with secondary HRP tagged antibodies in PBS-tween containing 5% skimmed milk powder for 1 hour at room temperature. The membranes were then washed five times with PBS-tween before the addition of ECL (BioRad, 1705062) and visualisation of a Licor C-digit.

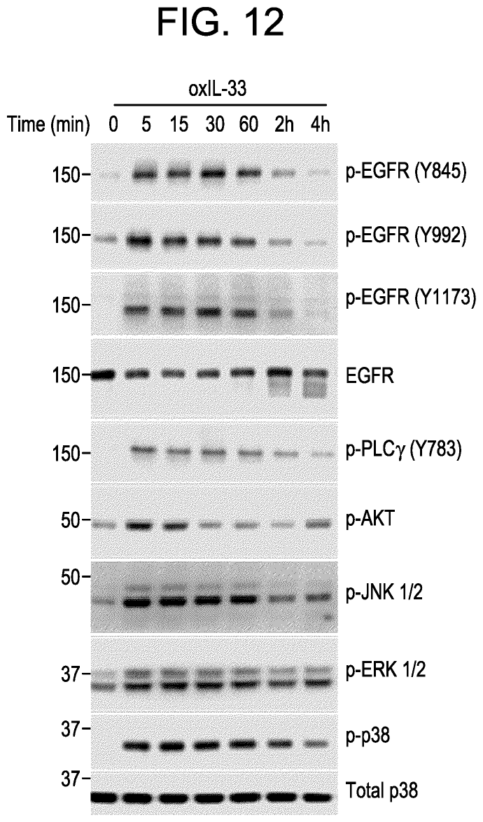

The results show that oxIL-33 activated several EGFR signaling components (FIG. 12)
8. Ox-IL-33 Induces STAT-5 Phosphorylation, Which is Blocked by EGFR-Neutralizing Ab

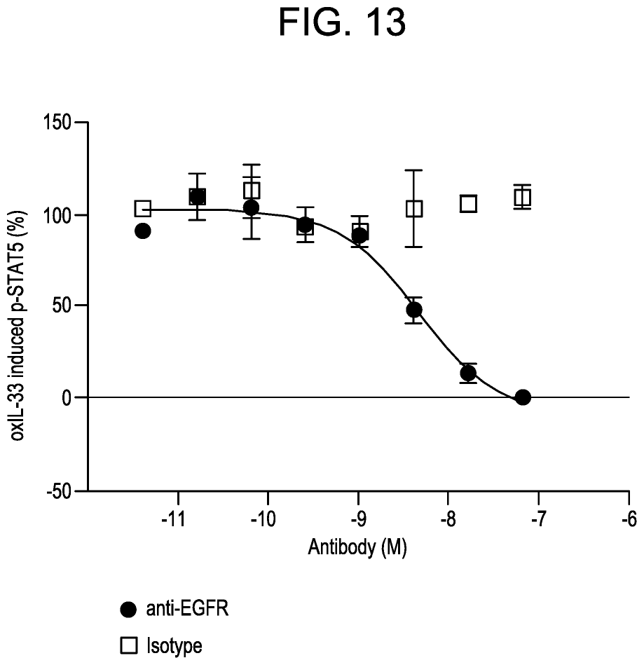

It was next sought to establish whether oxIL33-mediated STATS activation could be inhibited by preventing binding to EGFR. Briefly, A549 cells were cultured in RPMI GlutaMax medium supplemented with 1% Penicillin/Streptomycin and 10% FBS. Cells were harvested with accutase and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Anti-EGFR antibody (Clone LA1 (05-101, Millipore) or isotype control (MAB002, R&D Systems) was added in a dose dependent manner to the wells and the plate was returned to the incubator for 30 mins. The plates were then stimulated with oxidised IL-33 (30 ng/ml) for 30 mins before lysis using the phosho-STATS ELISA kit lysis buffer (85-86112-11, ThermoFischer Scientific) and developed following manufacturer's instructions before reading absorbance at 450 nM. As shown in FIG. 13, cells activated with oxIL-33-01 display phosphorylation of STATS, which decreases in the presence of anti-EGFR antibody (FIG. 13).

### Example 6

### Oxidised IL-33 Induces Complex Formation Between EGFR and RAGE

9. OxIL-33 Induces Complex Formation Between EGFR and RAGE

In order to understand how RAGE and EGFR are involved in promoting signaling of oxIL-33, immunoprecipitation experiments were performed to explore the signaling complex. Firstly, anti-EGFR antibodies were covalently coupled to Dynabeads. Two 100 μg vials of anti-EGFR antibodies (R&D systems, AF231) were incubated with 40 mg of Dynabeads (Thermo, 14311D) and covalently coupled as per the manufacturer's instructions. Following successful coupling the beads were resuspended in PBS at 30 mg/ml and kept at 4° C.

NHBEs were obtained from Lonza (CC-2540) and frozen vials seeded directly into 15 cm dishes (Thermo, 157150) at 1×10⁶ cells per dish. NHBEs were maintained in complete BEGM media (Lonza) according to the manufacturer's protocol for one month with a media change every three days until the cells reached confluency. The plates were incubated at 37° C., 5% CO₂ for the duration of this time. The day before stimulation, the plates were washed twice with 20 ml PBS before the addition of 15 ml starve media (BEGM (Lonza CC-3171) without supplement kit). The plates were then incubated at 37° C., 5% CO₂ for a further 18-24 hours before stimulation with media alone (unstimulated control), 30 ng/ml reduced IL-33-01, 30 ng/mL oxIL-33 or 30 ng/mL EGF and returned to 37° C., 5% CO₂ for 10 mins. Media was aspirated, and the plates were washed twice with ice-cold PBS before the addition of 1 ml lysis buffer (Abcam, ab152163) containing phosphatase and protease inhibitors (Thermo, 78440) per 15 cm dish. The cells were scraped into the lysis buffer before being transferred into 2 ml Protein LoBind tubes (Eppendorf, Z666513) and clarified by spinning at 14,000 rpm at 4° C. Protein concentration was determined using a BCA kit (Thermo, 23225) and all protein extracts were normalised to 3 mg/ml with lysis buffer. 6 mg of total protein extract was incubated in a clean 2ml LoBind tube with 100 μl of anti-EGFR Dynabeads (described above). The tubes were then placed on an end-over-end mixer at 4° C. for 5 h. Using a magnet (BioRad, 1614916) the Dynabeads were immobilised and the protein extract was aspirated and replaced with 2 ml wash buffer 1 (50 mM Tris-HCl pH 7.5 (Thermo, 15567027), 0.5% TritonX 100 (Sigma, X100), 0.3 M NaCl. This was repeated four more times. The beads were then washed a further ten times in the same manner with wash buffer 2 (50 mM Tris-HCl pH 7.5). After the final washing step, 50 μl of 1% Rapigest (w/v) (Waters, 186001861), in 50 mM Tris-HCl pH8.0, was added to the beads and heated at 60° C. for 10 min. The supernatant was then transferred to a new LoBind 2 ml tube. A further 100 μl of 50 mM Tris-HCl pH8.0 was added to the resin and mixed before it was combined with the first elution. TCEP (Sigma, 646547) was then added to a final concentration of 5 mM and the sample was heated at 60° C. for 10 min. The eluates were then alkylated by addition of iodoacetamide (Sigma, 16125) to 10 mM in the dark at room temperature for 20 min. The alkylation was quenched by the addition of DTT (Sigma, D5545) to 10 mM. Tris-HCl buffer 50 mM pH8.0 was then added to give a final sample volume of 500 μl. 0.5 μg of trypsin (Promega, V5111) per tube was added and samples were digested at 30° C. overnight at on a shaking platform at 400 rpm. The samples were then acidified with trifluoroacetic acid (Sigma, 302031) to a final concentration of 2.0% (v/v) and incubated at 37° C. for 1 h. Samples were then centrifuged at 14,000 rpm for 30 min and the supernatant was transferred to a new 2 ml LoBind tube. Samples were then processed through C18 columns (Thermo, 87784) as per the manufacturer's instructions. Samples were then dried using a speed-vac before being stored at −20° C. Samples were then analysed by peptide mass fingerprinting mass spectrometry (PMF-LC-MS). Scaffold software was used to analyse the results.

EGFR was detected similarly across all 4 conditions suggesting that the immunoprecipitation had worked well across all the samples. RAGE and IL-33 were detected in samples that had been treated with oxIL-33, in contrast to those treated with IL33-01 (IL-33) or EGF, suggesting that oxIL-33 and RAGE were associated with EGFR during signaling. Consistent with prior observations of EGFR activation in these cells with oxIL-33 and EGF, proteins previously reported to be involved in EGFR signaling and endocytosis were detected after stimulation with these ligands, but not reduced IL33-01 (Table 4).

Table 4 shows LCMS analysis of NHBE stimulated with reduced IL-33-01 (IL-33), oxIL-33 (oxidised IL-33-01) or EGF. IL-33 and RAGE are detected in complex with EGFR following stimulation with oxIL-33, but not after stimulation with reduced IL33-01 (IL-33) or EGF. Parentheses indicate the number of unique peptides identified for each protein.

TABLE 4

| Unstimulated | IL-33 | oxIL-33 | EGF |
|---|---|---|---|
| EGFR (63) | EGFR (62) | EGFR (60) | EGFR (57) |
| — | — | IL-33 (11) | — |
| — | — | RAGE (11) | — |
| — | — | AP-2α1 (20) | AP-2α1 (14) |
| — | — | AP-2α2 (16) | AP-2α2 (10) |
| — | — | AP-2β (15) | AP-2β (16) |
| — | — | AP-2μ (20) | AP-2μ (20) |
| — | — | AP-2σ (10) | AP-2σ (11) |
| — | — | CBL-B (5) | CBL-B (4) |

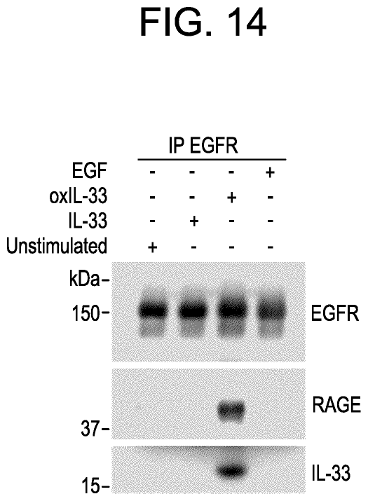

To confirm these observations, Immunoprecipitation and Western blotting was also performed on cell lysates prepared according to the above protocol. Following NHBE protein extract concentration determination, 3 mg of total protein was incubated in a 1.5 ml tube with 6 μg of anti-EGFR antibody (R&D systems, AF231) and placed on an end-over-end mixer at 4° C. for 2.5 h. 1.5 mg of protein A/G magnetic beads (Thermo, 88802) were then added to each tube and the tubes were then returned to 4° C. for another 1 h with mixing. The beads were then collected with a magnet (BioRad, 1614916) and washed three times with 500 μl of (50 mM Tris (pH 7.5), 1% TritonX and 0.25 M NaC1) and once with 500 μl of 10 mM Tris (pH 7.5). The proteins were then released from the magnetic beads using 35 μl of LDS sample buffer (Thermo, NP0008) with reducing agent (Thermo, NP0004) and heating at 95° C. for 5 minutes. Solutions were transferred to new 1.5 ml tubes and 10 μl of sample along with 5 μl of protein ladder (BioRad, 1610374) was run on a 4-12% SDS-PAGE gel (Thermo, NWO4127BOX) in IVIES running buffer (B0002). Gels were transferred onto PVDF membranes (BioRad, 1704156) using a Transblot Turbo (BioRad). PVDF membranes were blocked in PBS-tween solution containing 5% skimmed milk powder (Marvel) for 10 minutes. Membranes were then incubated with primary antibodies (anti-EGFR (Cell Signaling Technology, 2232), anti-RAGE (Cell Signaling Technology, 6996) or anti-IL-33 (R&D systems, AF3625) in PBS-tween containing 5% BSA over night at 4° C. The membranes were then washed five times with PBS-tween and then incubated with anti-rabbit secondary HRP tagged antibodies (Cell Signalling Technology, 7074) or anti-goat secondary HRP tagged antibodies (R&D systems, HAF109) in PBS-tween containing 5% skimmed milk powder for 1 hour at room temperature. The membranes were then washed five times with PBS-tween before the addition of ECL (BioRad, 1705062) and visualisation of a Licor C-digit. Western blotting confirmed that RAGE coprecipitated with EGFR in the presence of oxIL-33 whereas no RAGE was detected with EGF stimulation (FIG. 14). These findings reveal that RAGE and EGFR are a functional part of the oxidized IL-33 signaling complex.
10. RAGE is Required for oxIL-33 to Form a Complex with EGFR

The experiments described above have shown that oxIL-33 is a ligand for a complex of the EGF Receptor (EGFR), which results in downstream signaling. The experiments in this section are designed to determine whether oxIL-33 is a direct binding ligand for either RAGE or EGFR. To understand more about the formation of the signaling complex and assess whether oxIL-33 directly interacts with EGFR, an ELISA format was used to explore binding of oxIL-33 to RAGE, ST2-Fc and EGFR.

Proteins and Modifications: Proteins containing the Avitag sequence motif (GLNDIFEAQKIEWHE SEQ ID NO:46) were biotinylated using the biotin ligase (BirA) enzyme (Avidty, Bulk BirA) following the manufacturer's protocol. All modified proteins without Avitag used herein were biotinylated via free amines using EZ link Sulfo-NHS-LC-Biotin (Thermo/Pierce, 21335) following manufacturer protocols. Table 5 is the list of biotinylated proteins used.

TABLE 5

Reagent
Biotinylated EGF (Thermo)
Avitag-Human ASGPR
Avitag_IL-33-01 (reduced IL-33)
Avitag_IL-33-01 (oxidised IL-33)
Avitag_IL-33-16
HMGB1

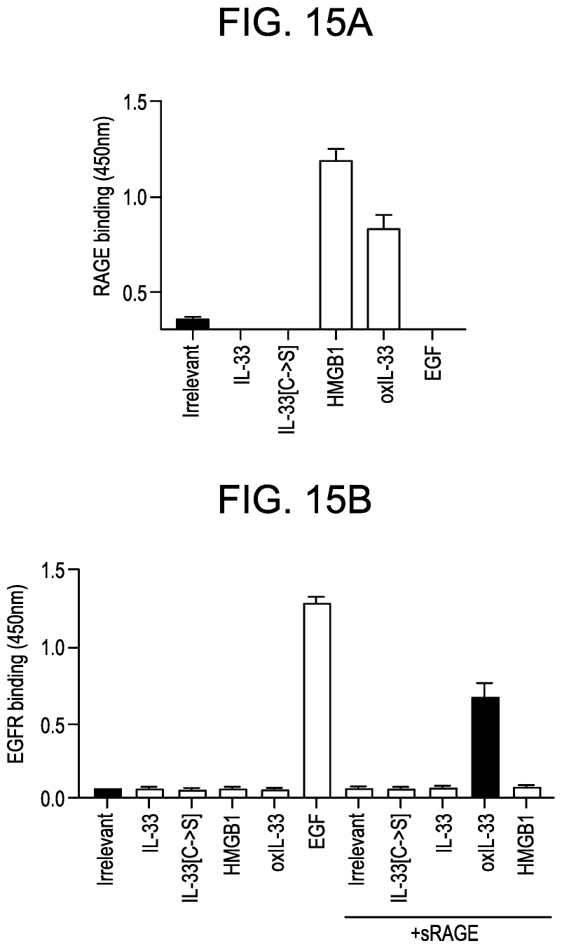

Streptavidin plates (Thermo Scientific, AB-1226) were coated with 100μ1/well of biotinylated antigen (10 μg/ml in PBS) at room temperature for 1 hour. Plates were washed 3×with 200 μl PBS-T (PBS +1% (v/v) Tween-20) and blocked with 300 μl/well blocking buffer (PBS with 1% BSA (Sigma, A9576)) for 1 hour. Plates were washed 3×with PBS-T. RAGE-Fc (R&D Systems #1145-RG) or ST2-Fc (R&D Systems #523-ST) were diluted to 10 μg/mL in PBS in blocking buffer, added to the relevant wells and incubated at room temperature for 1 hour. Alternatively, 100 μl of EGFR-Fc (R&D Systems #344-ER-050) at 10 μg/mL in PBS was added in the presence or absence of untagged RAGE (Sino Biological, 11629-HCCH) at 10 μg/mL in PBS for 1 hour. Plates were washed with 200 μl PBS-T three times. Then RAGE-Fc, ST2-Fc and EGFR-Fc were detected with anti-human IgG HRP (Sigma A0170, 5.1 mg/mL) diluted 1:10000 in blocking buffer, 100 μl/well for 1 hour at room temperature. Plates were washed 3×with PBS-T and developed with TMB, 100 μl/well (Sigma, T0440). The reaction was quenched with 50 μl/well 0.1 M H2504. Absorbance was read at 450nm on the Cytation Gen5 or similar equipment. The results show that oxIL-33 displayed a clear interaction with RAGE (FIG. 15A) whereas direct binding of oxIL-33 to EGFR was negligible (FIG. 15B). EGFR binding to oxIL-33 was observed only by the addition of sRAGE to this assay (FIG. 15B). This could not be recapitulated if oxIL-33 was substituted for a bona fide RAGE agonist, HMGB1 (FIG. 15B).

The need of RAGE in EGFR signaling triggered by oxIL-33 was further confirmed making use of RAGE-deficient cell lines. A RAGE knockout A549 cell line was generated as follows:

A mammalian plasmid was generated containing expression vectors for red fluorescent protein (RFP), guide RNA targeted to Exon 3 of AGER (TGAGGGGATTTTCCGGTGC SEQ ID NO:47) and Cas9 endonuclease. A549 conditioned media was generated by growing A549 cells in F12K nut mix (Gibco, supplemented with 10% FBS and 1% Penicillin/Streptomycin) in T-175 flasks for two days. Spent media was taken off the A549s, filtered, and diluted five-fold in fresh Gibco F12K nut mix (supplemented with 20% FBS and 1% Penicillin/Streptomycin). A549s were seeded into three T-75 flasks at 2×10⁵ cells/ml in 15 ml total and placed in a 37° C., 5% CO₂ incubator overnight. Transfection mix was prepared using 1.6 ml of F12K nut mix (supplemented with 1% Penicillin/Streptomycin) with 8 μg of the AGER guide RNA plasmid and 22.5 μg PEI (Polysciences, 23966-2). The mix was then vortexed for 10 seconds and left at room temperature for 15 mins. 0.75 ml of the transfection mix was then added to each T-75 flask. The flasks were returned to the incubator for two days. The A549 cells were then detached using Accutase and transferred into PBS containing 1% FBS and single cell sorted on an Aria cell sorter (BD) based on expression of RFP into a 96-well dish. The cells were fed every 3-5 days with conditioned media. Once cells became over 50% confluent, they were transferred to 24-well plates and grown up. This process of upscaling continued until each successful clone was split into T15 flasks. Cells were then split into 12 well plates and grown until over 50% confluent before analysis genomic PCR for successful knockouts. Cells were lysed in 100 μl DNA lysis buffer (Viagen Bitoech, 301-C, supplemented with 0.3 mg/ml proteinase K) per well. These samples were incubated at 55° C. for 4 hours followed by 15 min at 85° C. PCR of RAGE was performed with forward and reverse primers having the following sequences: forward — gttgcagcctcccaacttc (SEQ ID NO:48), reverse — aatgaggccagtggaagtca (SEQ ID NO:49). The reaction and cycling was set up as follows in a 50 μl reaction volume [25 μl Q5 polymerase mix, 2.5 μl forward primer (10 μiM stock), 2.5 μl reverse primer (10 μM stock), 2 μl of template DNA lysate, 18 μl nuclease-free water]. The PCR reaction was run with initial denaturation at 98° C. for 30 seconds, followed by 35 cycles of 98° C. for 5 seconds, 57° C. for 10 seconds and 72° C. for 20 seconds before a final step at 72° C. for 2 minutes. 4 μl of the PCR product was mixed with 6 μl nuclease-free water and 2 μl of 6×DNA loading buffer (Thermo Scientific, R0611). Samples were run on a 1% agarose gel (1:10000 SYBR safe) at 90V for 1 hour before visualisation on Versadoc Imager. The remainder of the PCR products were then cleaned up with the QIAquick PCR purification kit (Qiagen, 28104), following the manufacturer's protocol. DNA-50 concentration was measured using a nanodrop. Several clones (selected from results) were sent for in-house sequencing. Results showed successful insertion of stop codon in clones RAGE09 and RAGE10.

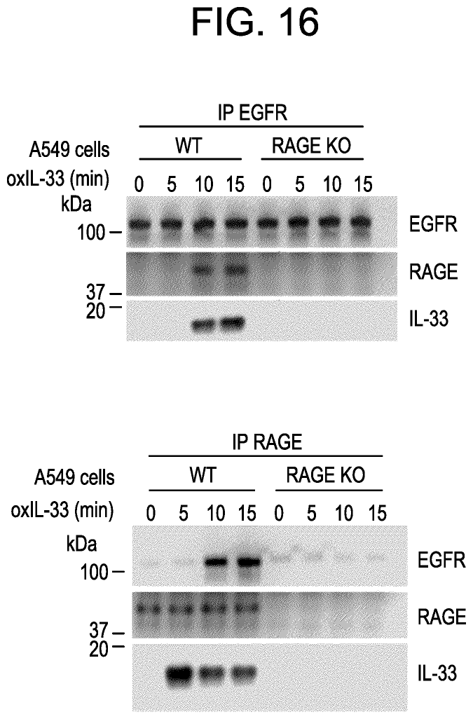

In order to ascertain the essentiality of RAGE to oxIL-33-mediated EGFR signaling, immunoprecipitation and Western blotting were then performed on A549 and the RAGE-deficient A549 cells. Briefly, cell lines were activated at various time points (0-15 minutes) with oxIL-33. Subsequent immunoprecipitation of EGFR or RAGE was followed by western blotting with anti-RAGE, anti-EGFR and anti-IL-33 following the relevant experimental protocols detailed in section 9. The results show the essential role of RAGE in the formation of a complex with oxIL-33 and EGFR (FIG. 16)
11. Oxidised IL-33 Induces STAT5 Phosphorylation Which is Blocked by RAGE, but not ST2 Neutralizing Antibody

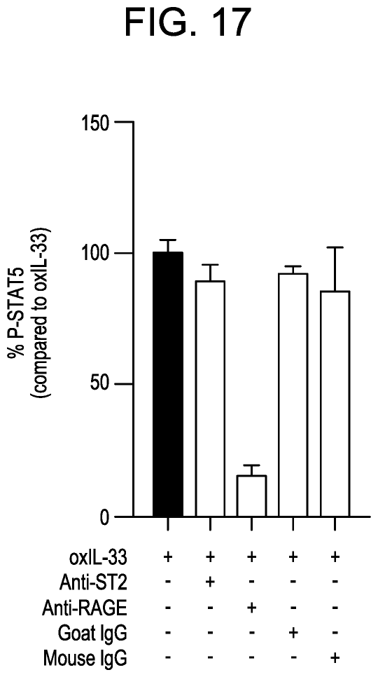

To confirm the importance of RAGE over ST2 in oxIL-33 signaling, blocking antibodies were tested. Briefly, A549s were cultured in RPMI GlutaMax medium supplemented with 1% Penicillin/Streptomycin and 10% FBS. Cells were harvested with accutase and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Anti-RAGE (M4F4; WO 2008137552); Anti-ST2 (AF532; RnD Systems) or isotype control (MAB002, R&D Systems) was added in a dose dependent manner to the wells and the plate was returned to the incubator for 30 mins. The plates were then stimulated with oxidised IL-33 (30 ng/ml) for 30 mins before lysis using the phosho-STAT5 ELISA kit lysis buffer (85-86112-11, ThermoFisher Scientific) and developed following manufacturer's instructions before reading absorbance at 450 nM. As shown in FIG. 17, cells activated with oxIL-33-01 display phosphorylation of STATS which decreased in the presence of anti-RAGE but not anti-ST2 antibody (FIG. 17).

### Example 7

### oxIL-33 Signalling Via RAGE in PTEC Kidney Cells

Previous examples have shown that IL-33 expression is elevated in kidney disease. RAGE expression is also elevated. Blocking ST2 and RAGE signalling was shown to reduce UACR and kidney damage in mice models. It has also been shown that oxIL-33-mediated signalling is driven by complex formation with EGFR in epithelial cells. The experiments described below sought to determine whether the novel signalling pathway is also present in kidney epithelium.

The response to oxIL-33 was measured in PTEC, a human proximal tubule epithelial line.

Briefly PTEC (primary human tubular epithelial cells line) were grown to confluence and treated with kidney inflammatory mediators for 24 h. IL-33 was measured in cell lysates using mesoscale diagnostics assays in accordance with manufacturers protocols.

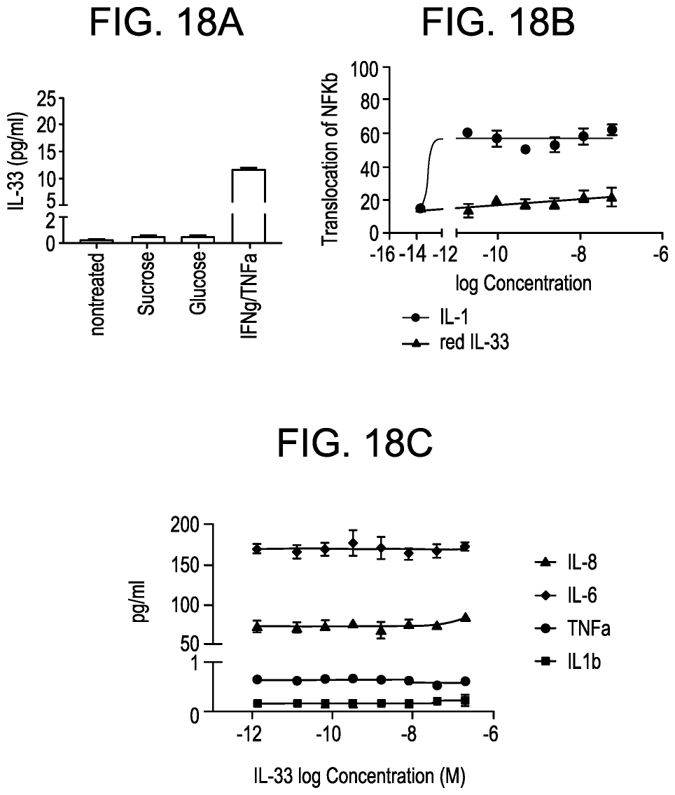

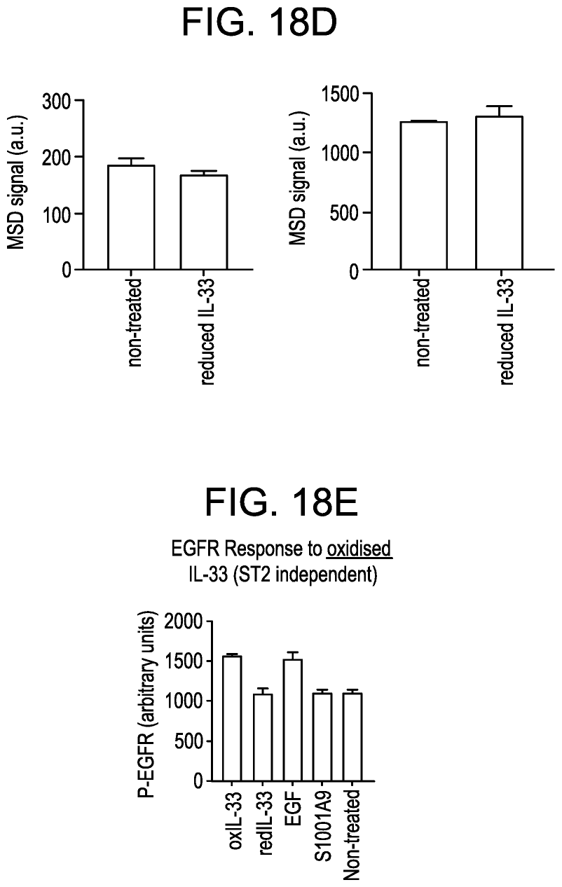

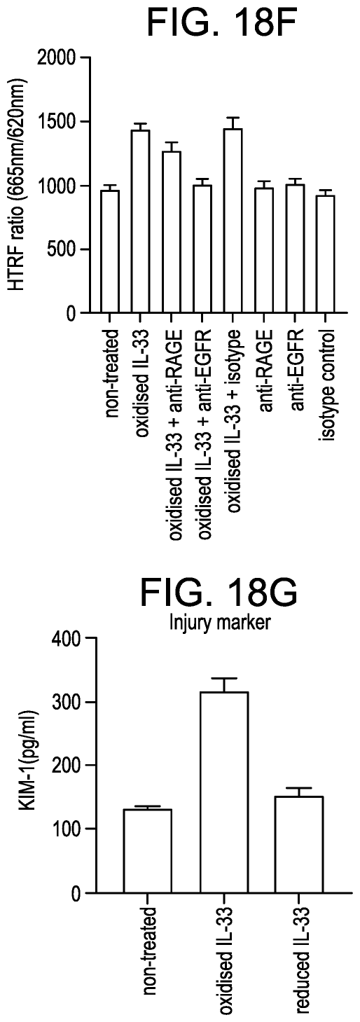

As shown in FIG. 18A, IL-33 intracellular concentrations increase when PTEC are treated with IFN-gamma and TNF, compared to when treated with sucrose or glucose controls. These results suggest the inflammatory mediators IFN gamma and TNF upregulate IL-33 production and secretion in PTEC.

PTEC were then treated with redIL-33 to examine potential autocrine or paracrine effects of redIL-33 on the inflammatory pathway via ST2 and NFkB. PTEC were grown to confluence and treated with dose concentrations of redIL-33 or IL-1 (obtained from peprotec 200-01B) as positive control . RedIL-33 was prepared as described in WO2016/156440. NFkB translocation to nucleus as a marker of activation was measured by immunofluorescence (following the method described in Noursadeghi et al J Immunol Methods 2008). FIG. 18B shows NFkB translocation in PTEC treated with increasing doses of IL-1 or redIL-33. These results show that red-IL33 invokes a lesser inflammatory response than IL-1 in PTEC.

This was further confirmed by analysing the dose-dependent release of inflammatory markers in PTECs in response to increasing concentrations of redIL-33. Briefly, Primary human PTEC (Lonza) were cultured to reach confluence, then seeded on the 24-well plates (no serum starvation) and stimulated for 24 hr with a full dose range of the IL-33 reduced form (doses from 12.8 pM to 200 nM). After that time, the supernatants were collected for a detection of proinflammatory cytokines. The detection of cytokines was performed by using the mesoscale diagnostic assay according to the manufacturer's instructions.

No dose-dependent increase in the level of the inflammatory cytokines IL-6, IL8, TNFa and IL1b was detected (FIG. 18C).

Similarly, the activation of MAP kinases were also analysed in PTECs upon treatment with reduced IL-33. Activation of MAP kinases is another cellular function that is regulated by the ST2 dependent pathway.

Briefly, Primary human PTEC were cultured to reach confluence, then seeded on the 24-well plates, no serum starvation was required for this study. Cells were stimulated for 30 min with the reduced form of IL-33 at a single concentration of 30 ng/ml for 30 min. After 30 min, cells were lysed to measure phosphorylation of MAP kinases (p38 and JNK). Phosphorylated MAP kinases were detected by using the mesoscale diagnostic assay according to the manufacturer's instructions.

The results show that PTECs do not exhibit increased MAP kinase signalling in response to reduced IL-33 (FIG. 18D), further illustrating that PTECs do not respond to reduced IL-33 via the classical ST2 signalling axis.

To examine if PTEC respond to signaling with oxIL-33, EGFR activation was measured after stimulation with oxIL-33 and redIL-33. OxIL-33 and redIL-33 were prepared as described in WO2016/156440 or above. Briefly, PTEC were grown to confluence and stimulated with oxIL-33 and redIL-33 for 10-15 min before measurement of RAGE/EGFR signalling by Homogeneous Time Resolved Fluorescence (HTRF).

An HTRF® assay is a homogeneous assay technology that utilises fluorescence resonance energy transfer between a donor and acceptor fluorophore that are in close proximity (Mathis, et al. Clin Chem 41(9):1391-7 (1995)). These assays were used to measure macromolecular interactions by directly or indirectly coupling one of the molecules of interest to a donor fluorophore, e.g. europium (Eu3+) cryptate, and coupling the other molecule of interest to an acceptor fluorophore e.g. XL665, (a stable cross linked allophycocyanin). In this donor/acceptor system, excitation of the cryptate molecule (at 337 nm) resulted in fluorescence emission at 620 nm. The energy from this emission was transferred to XL665 in close proximity to the Eu3+ cryptate, resulting in the emission of a specific long-lived fluorescence (at 665 nm) from the XL665. The specific signals of both the donor (at 620 nm) and the acceptor (at 665 nm) may be measured, allowing the calculation of a 665/620 nm ratio that compensates for the presence of coloured compounds in the assay.

Phospho-EGFR (Tyr1068) was detected in a sandwich assay format using two different specific antibodies, one labelled with Eu3+-Cryptate (donor) and the second with d2 (acceptor). When the dyes are in close proximity, the excitation of the donor with a light source (laser or flash lamp) triggers a Fluorescence Resonance Energy Transfer (FRET) towards the acceptor, which in turn fluoresces at a specific wavelength (665 nm). The specific signal modulates positively in proportion to phospho-EGFR (Tyr1068). Therefore, a FRET signal will only be observed when the EGFR signalling complex is activated.

As shown in FIG. 18E, ox-IL33 induces phosphorylation of EGFR (p-EGFR) in PTEC comparable to levels of the positive control (EGF) A natural ligand of RAGE (S1001A9), did not result in an increase in p-EGFR compared to untreated controls. Nor did redIL-33.

In order to confirm that the increase in p-EGFR is mediated by the oxIL33 RAGE-EGFR signalling pathway, PTEC were stimulated with oxidised IL-33 in presence of anti-RAGE and anti-EGFR antibodies.

Primary human PTEC were cultured to reach confluence, then seeded on the 96-well plates and serum starved overnight. Then cells were stimulated with a single dose of oxidised IL-33 (at 200 nM final) with/without anti-RAGE and anti-EGFR antibodies (at 10 ug/ml final). PTEC were preincubated with antibodies for 40 min and followed by a 10 min stimulation using oxidised IL-33. After that time (50 min in total), stimulations were terminated by lysing cells and processed for a detection of phosphorylated level of EGFR using the Homogeneous Time Resolved Fluorescence (HTRF). The assay details were described earlier. The assay was performed according to the Cisbio supplier's protocol.

The results show that blocking RAGE and EGFR reduced activation of EGFR by oxIL-33 (FIG. 18F). This indicates that EGFR activation in PTECs in response to oxIL33 is mediated by RAGE and EGFR.

These results show that oxIL-33, not redIL-33, activates RAGE/EGFR-dependent signalling in PTEC cells. This suggests that oxidized IL33 can mediate epithelial responses in tubular areas within kidney disease with raised IL33.

To assess the biological impact of oxIL33 signalling in the kidney epithelium, PTECs were stimulated with oxIL33 and redIL-33, and the release of Kidney-Injury-Molecule-1 (KIM-1) was measured. KIM-1 is a key marker of renal tubular injury (Han et al 2002, Kidney Int. 62(1)237-44).

Primary human PTEC were cultured to reach confluence, then seeded on the 24-well plates, serum starved overnight and stimulated with a single dose of oxidised IL-33 or and mutant reduced IL-33 (both at 1 ug/ml) (to specifically control for the effect that is specific to the oxidised form only). Stimulation went for 8 hr, after that time supernatants were collected for a subsequent detection of markers of renal injury KIM-1. The detection was performed using the mesoscale diagnostic assay according to the manufacturers protocol.

The results show that oxIL-33, but not the reduced IL-33 isoform upregulates KIM-1 (FIG. 18G), indicating that injury is a feature of activating the oxIL-33 signalling axis in PTECs.

### Example 8

### Blocking ST2 Dependent Signalling in Human Glomerular Endothelial Cells

The experiments described below establish whether different kidney cell types respond to redIL-33 signalling.

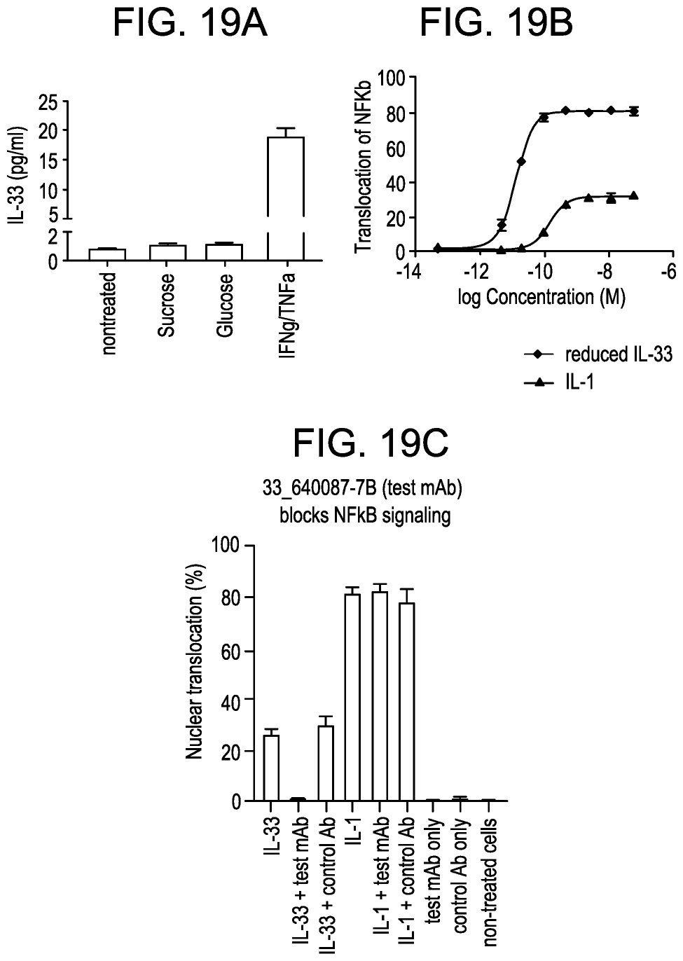

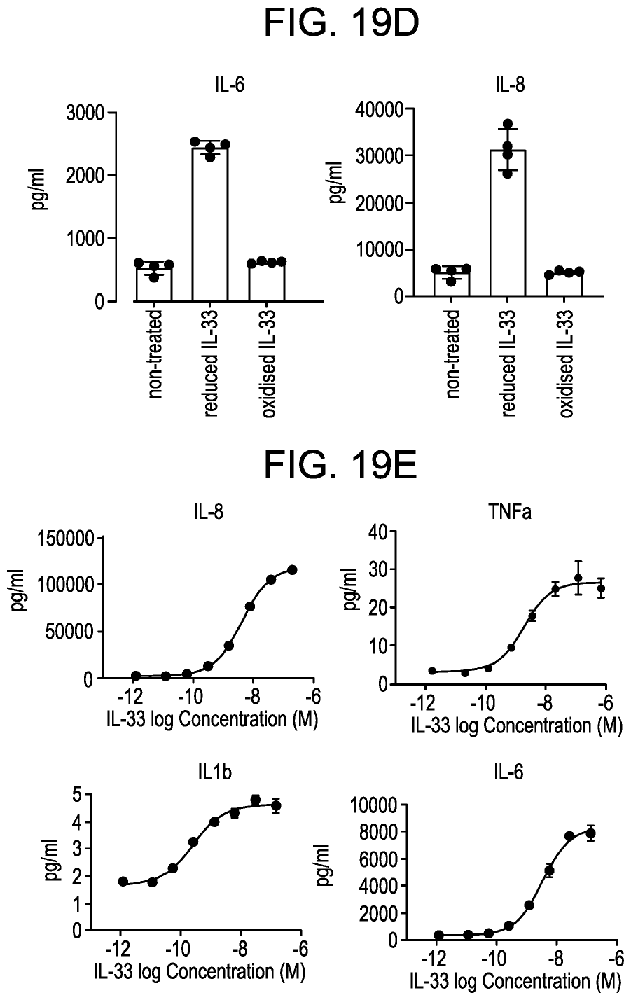

Primary human glomerular endothelial cells (GEnC) were grown to confluence and treated with kidney inflammatory mediators for 24 h. IL-33 was measured in cell lysates using MSD, as described in example 7. As shown in FIG. 19A, IL-33 intracellular concentrations increase when GEnC are treated with IFN-gamma and TNF, compared to when treated with sucrose or glucose controls. These data suggest inflammatory mediators IFN gamma and TNF upregulate IL-33 production and secretion in GEnC.

GEnC were then treated with redIL-33 to examine potential autocrine or paracrine effects of redIL-33 on the inflammatory pathway via ST2 and NFkB. GEnC were grown to confluence and treated with dose concentrations of redIL-33 or IL-1 (obtained from R&D Systems—cat no. 3625-IL-010, 201-LB-025) as positive control. RedIL-33 was prepared as described in WO2016/156440 or above. NFkB translocation to nucleus as a marker of activation was measured by immunofluorescence (following the method described in Noursadeghi et al J Immunol Methods 2008). FIG. 19B shows NFkB translocation in GEnC treated with increasing doses of IL-1 or redIL-33. These results show that at equivalent doses red-IL33 invokes a comparable inflammatory response to IL-1 in GEnC.

To examine effects of blocking IL-33 on ST2-dependent signalling in kidneys, GEnC were treated with the monoclonal antibody 33_640087-7B of WO2016/156440 (SEQ ID NO: 616 and SEQ ID NO: 618). Briefly, GEnC were grown to confluence and treated with IL-33 or control agents for 24 h with or without 0.0001-100 nM nM 33_640087-7B or isotype control. NFkB translocation was used a marker of ST2 signaling and endothelial activation and was assayed as described in the preceding examples.

As shown in FIG. 19C, redIL-33 induced NFkB translocation in GEnC treated with IL-33, which is inhibited by 33_640087-7B. Isotype control did not inhibit activation, nor did 33_640087-7B inhibit IL1-mediated NFkB signaling, which was used as a positive control.

Further experiments were performed to analyse the effect of redIL-33 stimulation of endothelial cells.

Primary human HGMEC (Cell Systems) were cultured to reach confluence, then seeded on 24-well plates and stimulated with a single dose (30 ng/ml) of reduced or the oxidised form of IL-33 for 24 hr. After that time, the supernatants were collected for a detection of proinflammatory IL-6 and IL-8 cytokines. The detection of cytokines was achieved by using the mesoscale diagnostic assay according to the manufacturer's instructions.

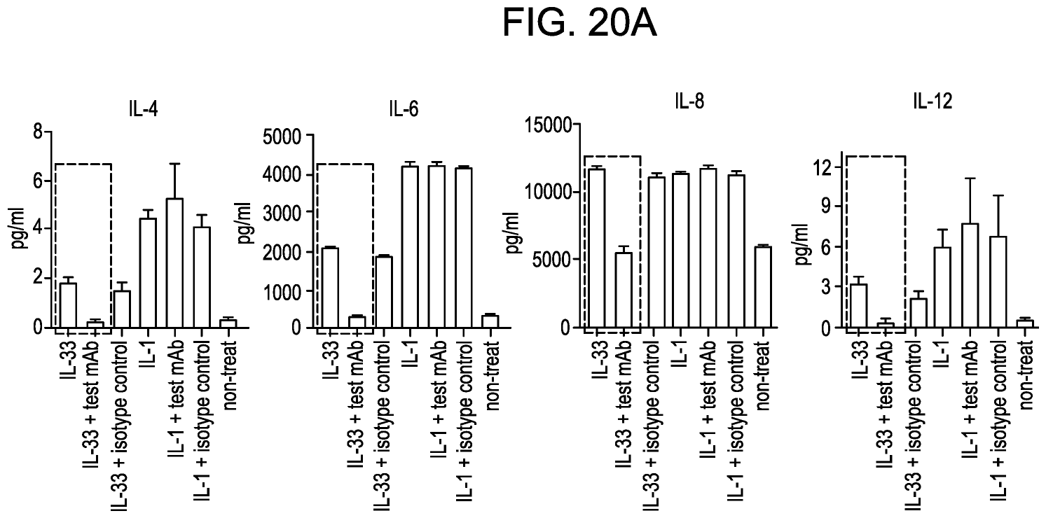

The results show that redIL-33 induces release of IL-6 and IL-8 (FIG. 20D). oxIL-33 did not induce IL-6 or IL-8 release.

The dose-dependent release of inflammatory cytokines IL-8, TNFa, IL1b and IL-6 after redIL-33 stimulation in HGMEC was also measured. Primary human HGMEC (Cell Systems) were cultured to reach confluence, then seeded on 24-well plates and stimulated for 24 hr with a full dose range of the reduced form of IL-33 (dose from 200 nM to 12.8 pM). After that time, the supernatants were collected for a detection of proinflammatory cytokines. The detection of cytokines was achieved by using the mesoscale diagnostic assay according to the manufacturer's instructions.

The results show that secretion of IL-1b, IL-6, IL-8 and TNFa from endothelial cells is specific to redIL-33 in a dose dependent manner.

To further explore redIL-33-induced glomerular endothelial cell NFkB activation in-vitro, the production of inflammatory cytokines in GEnC was measured after incubation with redIL-33 incubation for 24 h.

As above, GEnC were grown to confluence and incubated with IL33 or positive controls with or without 33_640087-7B. Cytokine levels were measured from supernatants using mesoscale diagnostics (MSD) assays in accordance with manufacturers protocols.

The ability of 33-640087_7B to inhibit activation of MAP kinase signalling was also measured in primary human endothelial cells. Primary human HGMEC (Cell Systems) were cultured to reach confluence, then seeded on the 24-well plates and stimulated for 30 min with a single dose of the redIL-33 at 30 ng/m with/without 33-640087_7B at lug/ml. After 30 min, cells were lysed to measure phosphorylation of MAP kinases and blocking effect of 33-640087_7B. MAP kinases were detected by using the mesoscale diagnostic assay according to the manufacturer's instructions.

The results show that 33 640087-7B significantly inhibits the secretion of IL-4, IL-6, IL-8 and IL-12, as measured from GEnC supernatant after 24 h (FIG. 20A). Furthermore, 33_640087-7B inhibits phosphorylation of MAP kinases p38 and JNK (FIG. 20B).

This demonstrates that IL-33 antagonists could be used to reduce or inhibit inflammation in the kidney mediated by IL-33/ST2 signalling. This may be useful in the treatment of diseases with abnormal inflammation in the kidney, such as that diabetic kidney disease.

### Example 9

### IL33 Signalling in Human Primary Mesangial Cells

Given that different isoforms of IL-33 have been shown to have differential pathological effects in kidney epithelial and endothelial cells, IL-33 signalling in mesangial cells was also analysed.

Mesangial cells are specialised cells that are another major component of kidney glomeruli. The primary function of mesangial cells is to remove trapped residues and aggregated protein from the basement membrane, thus keeping the filter free of debris. Diabetic kidney disease is characterised by progressive mesangial expansion and matrix deposition leading to glomerular hypertrophy and glomerulosclerosis which ultimately occludes glomerular capillaries and impairs kidney function.

Mesangial cells were stimulated with a range of chronic kidney disease stressors to establish whether they inducelL-33 expression.

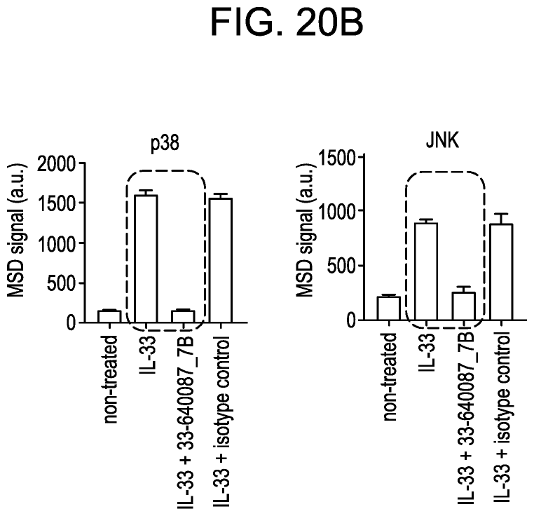

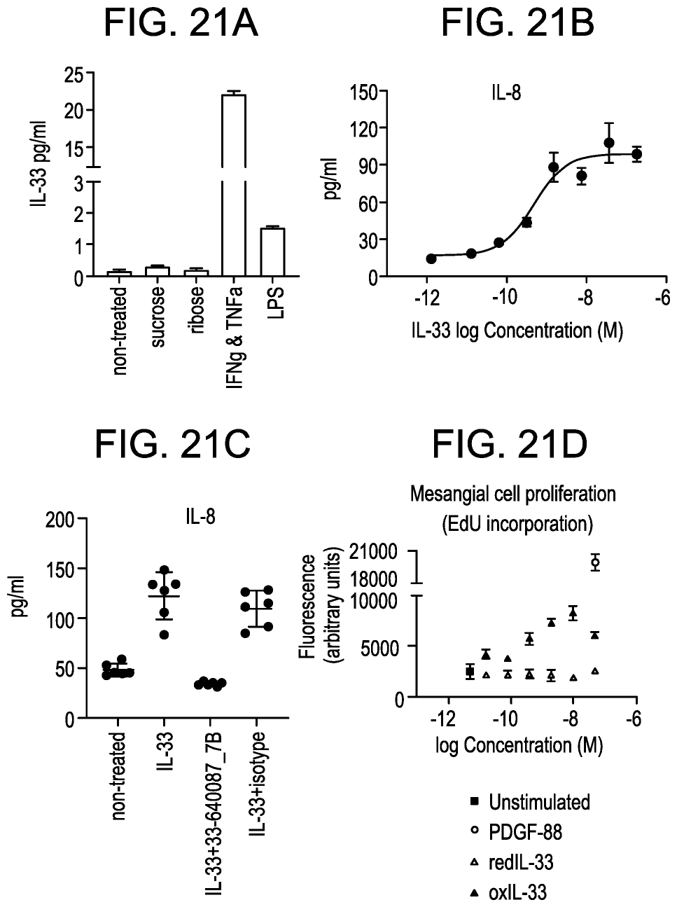

Primary human mesangial cells (Lonza) were grown to confluence, then seeded on 6well plates and treated with kidney inflammatory mediators for 24 h. IL-33 was measured in cell lysates using MSD, as described in examples above. As shown in FIG. 21A, IL-33 intracellular concentrations are upregulated when mesangial cells are treated with IFN-gamma and TNF-alpha, compared to other stressors.

The autocrine and paracrine effects of IL-33 expression within mesangial cells was next tested. The dose-dependent release of IL-8 from primary human mesangial cells upon treatment with redIL-33 was analysed.

Primary mesangial cells (Lonza) were grown to confluence, then seeded on 24-well plates and treated with a full dose range of the reduced form of IL-33 (dose from 200 nM to 12.8 pM) for 24 hr. Supernatants collected for detection of proinflammatory IL8. IL-8 was detected by using the mesoscale diagnostic assay according to the manufacturer's instructions. For blocking experiments, primary mesangial cells (Lonza) were grown to confluence, then seeded on 24-well plates and treated with a single dose of the reduced IL-33 (30 ng/ml) with/without 33-640087_7B at 1 ug/ml for 24 hr. Supernatants collected for detection of proinflammatory IL8. IL8 was detected by using the mesoscale diagnostic assay according to the manufacturer's instructions.

The results indicate that IL-8 release is dose-dependent (FIG. 21B). The release of IL-8 is inhibited by the presence of 33_640087_7B (FIG. 21C). Thus, IL-33 antagonists may be useful to inhibit autocrine or paracrine inflammatory response mediated by reduced IL-33 in mesangial cells.

To investigate the possible contribution of IL-33 signalling to mesangial expansion, which is observed in DKD, primary human mesangial cells were stimulated with IL-33 and proliferation of these cells was measured in vitro. In brief, human primary mesangial cells were grown to confluence, seeded in 96 well plates, then starved for 24 h and treated with dose concentrations of redIL-33, oxIL-33 or PDGF-BB as positive control for 18 h. Afterwards, cells were pulsed with a 10 uM EdU solution for an extra 4 h. EdU exposure allows for the direct measurement of cells synthesizing DNA. EdU incorporation was assessed by using the Amplex™ UltraRed reagent and measuring fluorescence emission following the manufacturer's instructions.

As shown in FIG. 21D, oxIL-33 induces proliferation of human mesangial cells in dose-dependent manner. These results suggests that oxIL-33, but not redIL-33, might be involved in mesangial cell expansion during progression of diabetic kidney disease.

### Example 10

### OxIL-33 Impairs Scratch Wound Repair Response in Submerged Monolayer Epithelial Cultures

oxIL-33 impairs scratch wound closure in A549 and NHBE cells, in contrast to EGF A549 epithelial cells were obtained from ATCC and cultured in RPMI GlutaMax medium supplemented with 1% Penicillin/Streptomycin and 10% FBS. Cells were harvested with accutase (PAA, #L1 1-007) and seeded into 96 well plates at 5×10⁵/100 μl and incubated at 37° C., 5% CO₂ for 18-24 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Using a WoundMaker™ (Essen Bioscience), cells were scratched and then wells were washed 2× with 200 μl of PBS before addition of RPMI GlutaMax medium supplemented with 0.1% FBS (v/v) and 1% (v/v) Penicillin/Streptomycin containing the indicated stimulations; media alone (unstimulated control), 30 ng/ml reduced IL-33, 30 ng/mL oxidised IL-33 or 30 ng/mL EGF and returned to 37° C., 5% CO₂. Plates were placed into an IncucyteZoom for wound healing imaging and analysis over a 48 hour period. Relative Wound Density was calculated through the wound healing algorithm within the Incucyte Zoom software.

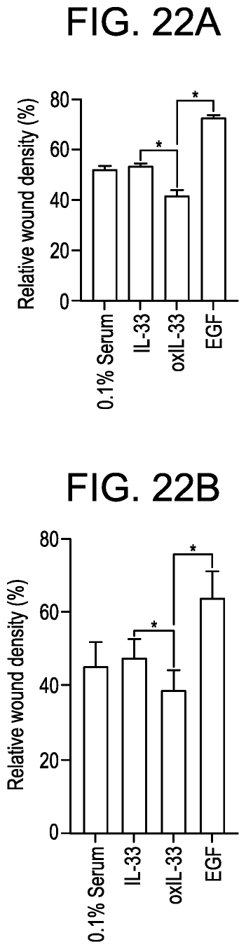

Normal human bronchial epithelial cells (NHBEs) (CC-2540) were obtained from Lonza and were maintained in complete BEGM media [BEGM (Lonza CC-3171) and supplement kit (Lonza CC-4175)] according to the manufacturer's protocol. Cells were harvested with accutase and seeded at 5×10⁵/100 μl in a 96-well ImageLock plate (Sartorius, 4379) in culture media. The plates were incubated at 37° C., 5% CO₂ for 18-24 hours. After this time, media was aspirated, and the cells were washed twice with 100μl PBS before the addition of starve media (BEGM (Lonza CC-3171) without supplement kit supplemented with 1% Penicillin/Steptomycin). The plates were then incubated at 37° C., 5% CO₂ for a further 18-24 hours before scratch wounding. Using a WoundMaker™ (Essen Bioscience), cells were scratched and then wells were washed 2× with 200 μl of PBS before addition of BEBM media (Lonza) supplemented with 0.1% FBS (v/v) and 1% (v/v) Penicillin/Streptomycin containing the indicated stimulations; media alone (unstimulated control), 30 ng/ml reduced IL-33, 30 ng/mL oxidised IL-33 or 30 ng/mL EGF and returned to 37° C., 5% CO₂. Plates were placed into an IncucyteZoom for wound healing imaging and analysis over a 48 hour period. Relative Wound Density was calculated through the wound healing algorithm within the Incucyte Zoom software. As shown in FIG. 22, oxIL-33 inhibited wound healing in submerged cultures of A549 cells (FIG. 22A) and NHBE cells (FIG. 22B), having an opposite effect to EGF where increased wound cell density is observed.
The Impairment of Scratch Wound Closure by Oxidised IL-33 Can be Prevented by Antibodies Neutralising RAGE or EGFR but Not ST2

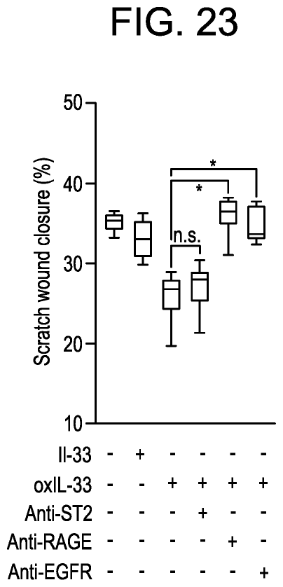

To understand whether these functional effects of oxIL-33 were mediated through RAGE/EGFR, the scratch assay was performed in NHBE cells as described above, but in the presence of antibodies that neutralised different receptor components. NHBE cells were treated with media alone (unstimulated control), reduced IL-33, or oxidised IL-33, in the presence of 10 μg/mL anti-ST2 (AF532, R&D Systems), anti-RAGE (M4F4, WO 2008137552) or anti-EGFR (Clone LA1, 05-101 Millipore). OxIL-33, but not reduced IL-33, inhibits scratch closure. This effect of oxIL-33 is reversed by anti-RAGE and anti-EGFR but not anti-ST2, again demonstrating that RAGE and EGFR are essential receptors involved in the oxidised IL-33 signalling pathway (FIG. 23).

A scratch wound assay may also be usedto examine epithelial cell response to injury noted in chronic kidney disease microenvironment. Briefly, RPTEC are seeded at 20,000-30,000/well in 96 well plate for 24 hrs (day 1). On day 2, PTEC cells are serum starved overnight. On day 3, scratch wound made using a wound maker (Essen Bioscience) and cells washed twice with PBS to remove detached cells. Stimuli are then applied (100 ul per well) and dilutions made in 0.1% serum medium, except cells in fully supplemented medium which are used as positive controls. Plates are inserted into Incucyte (Incucyte S3 2019A) and set up according to manufacturer's instructions to measure relative wound density at 0 hr (baseline) and then measurements taken every 4 hr for 4 days to measure the relative wound closure.

In conclusion, the data presented in the examples demonstrate that inflammatory mediators upregulate production of IL-33 in the kidney epithelium, endothelium and glomeruli. RedIL-33 appears to signal via an autocrine or paracrine mechanism through NFkB activation (ST2 dependent) in kidney endothelial cells. Red-IL33 signalling mediates proinflammatory cytokine secretion from glomerular endothelium, which is likely to exacerbate kidney injury in vivo. In addition, oxIL-33 activates the RAGE/EGFR signalling pathway (RAGE-dependent) in the kidney epithelium. RAGE/EGFR signalling is suspected to contribute to the pathophysiology of kidney injury. It has also been shown that RAGE expression is enhanced in the kidney in multiple pre-clinical models of kidney disease. Il-33 expression is enhanced in kidney disease. This means that concentrations of both redIL-33 and oxIL-33 may be enhanced within the kidney in CKD. Given that ST-2 appears to be surprisingly downregulated in the kidney during injury, the RAGE-EGFR/IL-33 system may contribute to IL-33-mediated pathologies in kidney disease.

ADDITIONAL SEQUENCES

| | |
|---|---|
| PAIR 1 HCDR1 | SEQ ID NO 37: SYAMS |
| PAIR 1 HCDR2 | SEQ ID NO 38: GISAIDQSTYYADSVKG |
| PAIR 1 HCDR3 | SEQ ID NO 39: QKFMQLWGGGLRYPFGY |
| PAIR 1 LCDR1 | SEQ ID NO 40: SGEGMGDKYAA |
| PAIR 1 LCDR2 | SEQ ID NO 41: RDTKRPS |
| PAIR 1 LCDR3 | SEQ ID NO 42: GVIQDNTGV |

N terminal His 10/Avitag/Factor Xa protease
cleavage site
SEQ ID NO 43: MHHHHHHHHHHAAGLNDIFEAQKIEWHEAAIEGR
IL-33-01
SEQ ID NO 44:
SITGISPITEYLASLSTYNDQSITFALEDESYEIYVEDLKKDEKKDKVLL
SYYESQHPSNESGDGVDGKMLMVTLSPTKDFWLHANNKEHSVELHKCEKP
LPDQAFFVLHNMHSNCVSFECKTDPGVFIGVKDNHLALIKVDSSENLCTE
NILFKLSET
IL-33-16
SEQ ID NO 45:
SITGISPITEYLASLSTYNDQSITFALEDESYEIYVEDLKKDEKKDKVLL
SYYESQHPSNESGDGVDGKMLMVTLSPTKDFWLHANNKEHSVELHKSEKP
LPDQAFFVLHNMHSNSVSFESKTDPGVFIGVKDNHLALIKVDSSENLSTE
NILFKLSET
Avitag sequence motif
SEQ ID NO 46: GLNDIFEAQKIEWHE
gRNA vector targeting RAGE exon 3
SEQ ID NO 47: TGAGGGGATTTTCCGGTGC
RAGE forward primer
SEQ ID NO 48: gttgcagcctcccaacttc
RAGE reverse primer
SEQ ID NO 49: aatgaggccagtggaagtca

1. An anti-IL-33 therapeutic agent for use in a method of treating kidney injury in a subject, wherein the anti-IL-33 therapeutic agent is to be administered to the subject to attenuate or inhibit IL-33-mediated ST2 signalling and IL-33-mediated RAGE signaling.

2. An anti-IL-33 therapeutic agent for use according to claim 1, wherein the IL-33-mediated RAGE signaling is IL-33-mediated RAGE-EGFR signaling.

3. An anti-IL-33 therapeutic agent for use according to claim 1, wherein the anti-IL-33 therapeutic agent attenuates or inhibits activity of reduced IL-33 protein (redIL-33) and thereby inhibits ST2 signalling.

4. An anti-IL-33 therapeutic agent for use according to any of claims 1 to 3, wherein the anti-IL-33 therapeutic agent attenuates or inhibits activity of oxidised IL-33 protein (oxIL-33) and thereby inhibits RAGE signaling.

5. An anti-IL-33 therapeutic agent for use according to any one of claims 1 to 4, wherein the kidney injury comprises inflammation.

6. An anti-IL-33 therapeutic agent for use according to claim 5, wherein the kidney injury is an inflammatory kidney injury.

7. An anti-IL-33 therapeutic agent for use according to claim 5 or 6, wherein the kidney injury is selected from diabetic kidney disease, fibrosis, glomerulonephritis (for example non-proliferative (such as minimal change glomerulonephritis, membrane glomerulonephritis, focal segmental glomerulosclerosis) or prolative (such as IgA nephropathy, membranoproliferative glomerulonephritis, post infectious glomerulonephritis, and rapidly progressive glomerulonephritis [such as Goodpastures syndrome and vasculitic disorders {which includes Wegners granulomatosis and microscopic polyangiitis}]), systemic lupus erythematosus, albuminuria, unilateral ureteral obstruction, Alport syndrome, polycystic kidney disease (PCKD), hypertensive glomerulosclerosis, chronic glomerulosclerosis, chronic obstructive uropathy, chronic tubulo-interstitial nephritis and ischemic nephropathy.

8. An anti-IL-33 therapeutic agent for use according to any one of claims 1 to 7, wherein the kidney injury is diabetic kidney disease.

9. An anti-IL-33 therapeutic agent for use according to any one of claims 1 to 8, wherein the anti-IL-33 therapeutic agent is selected from a chemical inhibitor and an antibody or antigen-binding fragment thereof.

10. An anti-IL-33 therapeutic agent for use according to claim 9, wherein the therapeutic agent comprises an antibody or antigen-binding fragment thereof.

11. An anti-IL-33 therapeutic agent for use according to claim 9 or 10, wherein the antibody or antigen-binding fragment thereof binds specifically to IL-33.

12. An anti-IL-33 therapeutic agent for use according to claim 11, wherein the antibody or antigen-binding fragment has the complementarity determining regions (CDRs) of a variable heavy domain (VH) and a variable light domain (VL) pair selected from Table 1.

13. An anti-IL-33 therapeutic agent for use according to claim 11 or 12, wherein the antibody or antigen-binding fragment thereof specifically binds to redIL-33 and attenuates or inhibits activity of redIL-33, thereby inhibiting ST2 signalling.

14. An anti-IL-33 therapeutic agent for use according to any of claims 11 to 13, wherein the antibody or antigen-binding fragment thereof prevents binding of oxidised IL-33 to RAGE, thereby inhibiting RAGE-EGFR signalling.

15. An anti-IL-33 therapeutic agent for use according to any one of claims 11 to 14, wherein the antibody or an antigen-binding fragment thereof binds to redIL-33 with a binding affinity of less than or equal to 100 pM, or less than or equal to 10 pM, for example less than or equal to 1 pM, such as 0.5 pM, in particular 0.05 pM (for example when measured using KinExA).

16. An anti-IL-33 therapeutic agent for use according to any one of claims 11 to 15, wherein the antibody or an antigen-binding fragment thereof binds to redIL-33 with a k(on) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹, in particular greater than or equal to 10⁷ M⁻¹ sec⁻¹.

17. An anti-IL-33 therapeutic agent for use according to any one of claims 11 to 16, wherein the antibody or an antigen-binding fragment thereof binds to redIL-33 with a k(off) less than or equal to 5×10⁻¹ sec⁻¹, 10⁻¹ sec⁻¹, 5×10⁻² sec⁻¹, 10⁻² sec⁻¹, 5 ×10⁻³ sec⁻¹ or 10⁻³ sec⁻¹, in particular less than or equal to 10⁻³ sec⁻¹.

18. An anti-IL-33 therapeutic agent for use according to any one of claims 11 to 17, wherein the antibody or an antigen-binding fragment attenuates or inhibits the activity of oxIL-33 and thereby inhibits RAGE signaling.

19. An anti-IL-33 therapeutic agent for use according to any one of claims 10 to 18, wherein the antibody or antigen-binding fragment comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

20. An anti-IL-33 therapeutic agent for use according to any one of claims 10 to 19, wherein the antibody or antigen-binding VH and VL of said antibody or antigen-binding fragment thereof comprise amino acid sequences at least 85%, 90%, 95%, 96%, 97%, 98% or 99% identical to SEQ ID NO: 1 and SEQ ID NO: 19, respectively.

21. An anti-IL-33 therapeutic agent for use according to claim 20, wherein the antibody or antigen-binding fragment comprises a VH having the sequence of SEQ ID NO: 1 and a VL having the sequence of SEQ ID NO:19.

22. An anti-IL-33 therapeutic agent for use according to any one of claims 10 to 21, wherein the antibody or an antigen-binding fragment thereof is a human antibody, a chimeric antibody, and a humanized antibody.

23. An anti-IL-33 therapeutic agent for use according to any one of claims 10 to 22, wherein the antibody or the antibody or an antigen-binding fragment thereof is a naturally-occurring antibody, an scFv fragment, an Fab fragment, an F(ab′)2 fragment, a minibody, a diabody, a triabody, a tetrabody, or a single chain antibody.

24. An anti-IL-33 therapeutic agent for use according to any one of claims 10 to 23, wherein the antibody or an antigen-binding fragment thereof is a monoclonal antibody.

25. An anti-IL-33 therapeutic agent for use of any of claims 2 to 24, wherein inhibition or attenuation of RAGE-EGFR signaling down-regulates or inhibits RAGE-EGFR mediated effects.

26. An anti-IL-33 therapeutic agent for use according to claim 25, wherein the RAGE-EGFR mediated effect comprises abnormal epithelium physiology, such as abnormal epithelium remodelling.

27. An anti-IL-33 therapeutic agent for use according to claim 25, wherein the RAGE-EGFR mediated effect comprises abnormal mesangial expansion.

28. An anti-IL-33 therapeutic agent for use according to claim 27, wherein abnormal mesangial expansion comprises abnormal mesangial cell proliferation.

29. An anti-IL-33 therapeutic agent for use according to any of claims 1 to 28, wherein inhibition or attenuation of ST2 signalling down-regulates or inhibits ST2 mediated effects.

30. An anti-IL-33 therapeutic agent for use according to claim 29, wherein the ST2 mediated effect is abnormal inflammation in the kidney.

31. An anti-IL-33 therapeutic agent for use of claim 30, wherein the abnormal inflammation is in the endothelium.

32. An anti-IL-33 therapeutic agent for use according to claim 31, wherein the abnormal inflammation comprises increased IL-4, IL-6, IL-8, IL-12, TNFa and/or IL1b secretion or expression, optionally increased IL-4, IL-6, IL-8 and/or IL-12 secretion or expression.

33. An anti-IL-33 therapeutic agent for use according to either claim 31 or 32, wherein the abnormal inflammation comprises MAP kinase activation.

34. An anti-IL-33 therapeutic agent for use according to claim 33, wherein MAP kinase activation comprises p38 or JNK kinase activation.

35. An anti-IL-33 therapeutic agent for use according to claim 30, wherein the abnormal inflammation is in the glomeruli.

36. An anti-IL-33 therapeutic agent for use according to claim 35, wherein the abnormal inflammation comprises increased IL-8 secretion or expression.

37. A method of treating kidney injury in a subject in need thereof, the method comprising administering to the subject an anti-IL-33 therapeutic agent to attenuate or inhibit IL-33-mediated ST2 signalling and IL-33-mediated RAGE signaling.

38. A method according to claim 37, wherein the IL-33-mediated RAGE signaling is IL-33-mediated RAGE-EGFR signaling.

39. A method according to claim 37 or 38, wherein the anti-IL-33 therapeutic agent attenuates or inhibits activity of reduced IL-33 protein (redIL-33) and thereby inhibits ST2 signalling.

40. A method according to claims 37 to 39, wherein the anti-IL-33 therapeutic agent attenuates or inhibits activity of oxidised IL-33 protein (oxIL-33) and thereby inhibits RAGE signaling.

41. A method according to any one of claims 37 to 40, wherein the kidney injury comprises inflammation.

42. A method according to claim 41, wherein the kidney injury is an inflammatory kidney injury.

43. A method according to claim 41 or 42, wherein the kidney injury is selected from diabetic kidney disease, fibrosis, glomerulonephritis (for example non-proliferative (such as minimal change glomerulonephritis, membrane glomerulonephritis, focal segmental glomerulosclerosis) or prolative (such as IgA nephropathy, membranoproliferative glomerulonephritis, post infectious glomerulonephritis, and rapidly progressive glomerulonephritis [such as Goodpastures syndrome and vasculitic disorders {which includes Wegners granulomatosis and microscopic polyangiitis}]), systemic lupus erythematosus, albuminuria, unilateral ureteral obstruction, Alport syndrome, polycystic kidney disease (PCKD), hypertensive glomerulosclerosis, chronic glomerulosclerosis, chronic obstructive uropathy, chronic tubulo-interstitial nephritis and ischemic nephropathy.

44. A method according to any one of claims 37 to 43, wherein the kidney injury is diabetic kidney disease.

45. A method according to any one of claims 37 to 44, wherein the anti-IL-33 therapeutic agent is selected from a chemical inhibitor and an antibody or antigen-binding fragment thereof.

46. A method according to claim 45, wherein the therapeutic agent comprises an antibody or antigen-binding fragment thereof.

47. A method according to claim 45 or 46, wherein the antibody or antigen-binding fragment thereof binds specifically to IL-33.

48. A method according to claim 47, wherein the antibody or antigen-binding fragment has the complementarity determining regions (CDRs) of a variable heavy domain (VH) and a variable light domain (VL) pair selected from Table 1.

49. A method according to claim 47 or 48, wherein the antibody or antigen-binding fragment thereof specifically binds to redIL-33 and attenuates or inhibits activity of redIL-33, thereby inhibiting ST2 signalling.

50. A method according to any of claims 47 to 49, wherein the antibody or antigen-binding fragment thereof prevents binding of oxidised IL-33 to RAGE, thereby inhibiting RAGE-EGFR signalling.

51. A method according to any one of claims 47 to 50, wherein the antibody or an antigen-binding fragment thereof binds to redIL-33 with a binding affinity of less than or equal to 100 pM, or less than or equal to 10 pM, for example less than or equal to 1 pM, such as 0.5 pM, in particular 0.05 pM (for example when measured using KinExA).

52. A method according to any one of claims 47 to 51, wherein the antibody or an antigen-binding fragment thereof binds to redIL-33 with a k(on) greater than or equal to 10⁵M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹, in particular greater than or equal to 10⁷M⁻¹sec⁻¹.

53. A method according to any one of claims 47 to 52, wherein the antibody or an antigen-binding fragment thereof binds to redIL-33 with a k(off) less than or equal to 5×10⁻¹ sec⁻¹, 10⁻¹ sec⁻¹, 5×10⁻² sec⁻¹, 10⁻² sec⁻¹, 5×10⁻³ sec⁻¹ or 10⁻³ sec⁻¹, in particular less than or equal to 10⁻³ sec⁻¹.

54. A method according to any one of claims 47 to 53, wherein the antibody or an antigen-binding fragment attenuates or inhibits the activity of oxIL-33 and thereby inhibits RAGE signaling.

55. A method according to any one of claims 47 to 54, wherein the antibody or antigen-binding fragment comprises a VHCDR1 having the sequence of SEQ ID NO: 37, a VHCDR2 having the sequence of SEQ ID NO: 38, a VHCDR3 having the sequence of SEQ ID NO: 39, a VLCDR1 having the sequence of SEQ ID NO: 40, a VLCDR2 having the sequence of SEQ ID NO: 41, and a VLCDR3 having the sequence of SEQ ID NO: 42.

56. A method according to any one of claims 47 to 55, wherein the antibody or antigen-binding VH and VL of said antibody or antigen-binding fragment thereof comprise amino acid sequences at least 85%, 90%, 95%, 96%, 97%, 98% or 99% identical to SEQ ID NO: 1 and SEQ ID NO: 19, respectively.

57. A method according to claim 56, wherein the antibody or antigen-binding fragment comprises a VH having the sequence of SEQ ID NO: 1 and a VL having the sequence of SEQ ID NO:19.

58. A method of any one of claims 47 to 57, wherein the antibody or an antigen-binding fragment thereof is a human antibody, a chimeric antibody, and a humanized antibody.

59. A method of any one of claims 47 to 57, wherein the antibody or the antibody or an antigen-binding fragment thereof is a naturally-occurring antibody, an scFv fragment, an Fab fragment, an F(ab′)2 fragment, a minibody, a diabody, a triabody, a tetrabody, or a single chain antibody.

60. A method of any one of claims 47 to 59, wherein the antibody or an antigen-binding fragment thereof is a monoclonal antibody.

61. The method of claims 48 to 60, wherein inhibition or attenuation of RAGE-EGFR signaling down-regulates or inhibits RAGE-EGFR mediated effects.

62. A method of claim 61, wherein the RAGE-EGFR mediated effect comprises abnormal epithelium physiology, such as abnormal epithelium remodelling.

63. A method according to claim 61, wherein the RAGE-EGFR mediated effect comprises abnormal mesangial expansion.

64. A method according to claim 63, wherein abnormal mesangial expansion comprises abnormal mesangial cell proliferation.

65. A method according to any of claims 47 to 64, wherein inhibition or attenuation or ST2 signalling down-regulates or inhibits ST2 mediated effects.

66. A method according to claim 65, wherein the ST2 mediated effect is abnormal inflammation in the kidney.

67. A method of claim 66, wherein the abnormal inflammation is in the endothelium.

68. A method according to claim 67, wherein the abnormal inflammation comprises increased IL-4, IL-6, IL-8, IL-12, TNFa and/or IL1b secretion or expression, optionally increased IL-4, IL-6, IL-8 and/or IL-12 secretion or expression.

69. A method according to claim 67, wherein the abnormal inflammation comprises MAP kinase activation.

70. A method according to claim 69, wherein MAP kinase activation comprises p38 or JNK kinase activation.

71. A method according to claim 66, wherein the abnormal inflammation is in the glomeruli.

72. An anti-IL-33 therapeutic agent for use according to claim 71, wherein the abnormal inflammation comprises increased IL-8 secretion or expression.


SEQUENCE LISTING (49 sequences)

SEQ ID NO: 1 - protein, 126 aa, Artificial Sequence
  PAIR 1 VH
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 2 - protein, 122 aa, Artificial Sequence
  PAIR 2 VH
EVQLVESGGGLVQPGGSLRLSCAASGFTFRSFAMSWVRQAPGKGLELVSDLRTSGGSTYYADSVKGRLTISRDNSKNTLYLQMNSLRAEDTAVYYCAKSHYSTSWFGGFDYWGQGTLVTVSS

SEQ ID NO: 3 - protein, 121 aa, Artificial Sequence
  PAIR 3 VH
QVQLQESGPGLVKPSETLSLTCTVSGGSISSYYWSWIRQPPGKGLELIGYIYYSGSTNYNPSLKSRVTISVDTSKNHFSLKLSSVTAADTAVYYCARSQYTSSWYGSFDIWGQGTMVTVSS

SEQ ID NO: 4 - protein, 122 aa, Artificial Sequence
  PAIR 4 VH
QVQLVQSGAEVKKPGASVKVSCKASGYTFNSYGISWVRQAPGQGLEWMGWISSHNGNSHYVQKFQGRVSMTTDTSTSTAYMELRSLRSDDTAVYYCARHSYTTSWYGGFDYWGQGTLVTVSS

SEQ ID NO: 5 - protein, 122 aa, Artificial Sequence
  PAIR 5 VH
EVQLVESGGGLVQPGGSLRLSCAASGFTFSSYALTWVRQAPGKGLEWVSFISGSGGRPFYADSVKGRFTISRDNSKNMLYLQMNSLRAEDTAIYYCAKSLYTTSWYGGFDSWGQGTLVTVSS

SEQ ID NO: 6 - protein, 122 aa, Artificial Sequence
  PAIR 6 VH
EVQLVESGGGLVQPGGSLRLSCAASGFTFSNYAMTWVRQAPGKGLEWVSTISGSGDNTYYADSVQGRFTISRGHSKNTLYLQMNSLRAEDTAVYYCAKPTYSRSWYGAFDFWGQGTMVTVSS

SEQ ID NO: 7 - protein, 122 aa, Artificial Sequence
  PAIR 7 VH
EVQLVESGGNLEQPGGSLRLSCTASGFTFSRSAMNWVRRAPGKGLEWVSGISGSGGRTYYADSVKGRFTISRDNSKNTLYLQMNSLSAEDTAAYYCAKDSYTTSWYGGMDVWGHGTTVTVSS

SEQ ID NO: 8 - protein, 116 aa, Artificial Sequence
  PAIR 8 VH
EVQLLESGGGLVQPGGSLRLSCAASGFTFSDYYMNWVRQAPGKGLEWVSSISRYSSYIYYADSVKGRFTISRDNSKNTLYLQMNSLRAEDTAVYYCARDIGGMDVWGQGTLVTVSS

SEQ ID NO: 9 - protein, 119 aa, Artificial Sequence
  PAIR 9 VH
EVQLLESGGGLVQPGGSLRLSCAASGFTFSNYYMHWVRQAPGKGLEWVSSISARSRYHYYADSVKGRFTISRDNSKNTLYLQMNSLRAEDTAVYYCARLATRHNAFDIWGQGTLVTVSS

SEQ ID NO: 10 - protein, 119 aa, Artificial Sequence
  PAIR 10 VH
EVQLLESGGGLVQPGGSLRLSCAASGFTFSNYYMHWVRQAPGKGLEWVSSISARSSYIYYADSVKGRFTISRDNSKNTLYLQMNSLRAEDTAVYYCARLATRNNAFDIWGQGTLVTVSS

SEQ ID NO: 11 - protein, 119 aa, Artificial Sequence
  PAIR 11 VH
EVQLLESGGGLVQPGGSLRLSCAASGFTFSRYYMHWVRQAPGKGLEWVSSISAQSSHIYYADSVEGRFTISRDNSKNTLYLQMNSLRAEDTAVYYCARLATRQNAFDIWGQGTLVTVSS

SEQ ID NO: 12 - protein, 119 aa, Artificial Sequence
  PAIR 12 VH
EVQLLESGGGLVQPGGSLRLSCAASGFTFSNYYMHWVRQAPGKGLEWVSSISARSSYLYYADSVKGRFTISRDNSKNTLYLQMNSLRAEDTAVYYCARLATRHVAFDIWGQGTLVTVSS

SEQ ID NO: 13 - protein, 139 aa, Artificial Sequence
  PAIR 13 VH
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 14 - protein, 123 aa, Artificial Sequence
  PAIR 14 VH
EVQLVETGGGLIQPGGSLRLSCAASGFTFSSYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 15 - protein, 123 aa, Artificial Sequence
  PAIR 15 VH
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTLHGIRAAYDAFIIWGQGTLVTVSS

SEQ ID NO: 16 - protein, 123 aa, Artificial Sequence
  PAIR 16 VH
EVQLVETGGGLIQPGGSLRLSCAASGFTFSFYAMSWVRQAPGKGLEWVSAISGSGGSTYYADSVKGRFTISRDNSKNTLYLQMNSLRAEDTAVYYCARTIHGIRAAYDAFIIWGQGTLVTVSS

SEQ ID NO: 17 - protein, 121 aa, Artificial Sequence
  PAIR 17 VH
EVQLVESGGGLVQPGGSLRLSCAASGFTFSSYWMYWVRQAPGKGLEWVAAITPNAGEDYYPESVKGRFTISRDNAKNSLYLQMNSLRAEDTAVYYCARGHYYYTSYSLGYWGQGTLVTVSS

SEQ ID NO: 18 - protein, 119 aa, Artificial Sequence
  PAIR 18 VH
EVQLVESGGGLVQPGGSLRLSCAASGFTFSSFSMSWVRQAPGKGLEWVATISGGKTFTDYVDSVKGRFTISRDDSKNTLYLQMNSLRAEDTAVYYCTRANYGNWFFEVWGQGTLVTVSS

SEQ ID NO: 19 - protein, 106 aa, Artificial Sequence
  PAIR 1 VL
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 20 - protein, 107 aa, Artificial Sequence
  PAIR 2 VL
DIQMTQSPSSVSASVGDRVTITCRASQGFSSWLAWYQQKPGKAPKLLIYAASSLQSGVPSRFSGSGSGTDFTLTITNLQPEDFATYYCQQANSFPLTFGGGTKVEIK

SEQ ID NO: 21 - protein, 107 aa, Artificial Sequence
  PAIR 3 VL
DIQMTQSPSSVSASVGDRVTITCRASQGISTWLAWFQQKPGKAPKLLIYAASTLQGGVPSRFSGSGSGPEFTLTISSLQPEDFATYYCQQANSFPWTFGQGTKVEIK

SEQ ID NO: 22 - protein, 107 aa, Artificial Sequence
  PAIR 4 VL
DIQMTQSPSSVSASVGDRVTITCRASQGFSSWLAWYQQKPGKAPQLLIYAASSLQSGVPSRFSGSGSGSDFTLTISSLQPEDFATYYCQQANSFPLTFGGGTKVEIK

SEQ ID NO: 23 - protein, 107 aa, Artificial Sequence
  PAIR 5 VL
DIQMTQSPSSVSASVGDRVTITCRASQGVVSWLAWYQQKPGKAPKLLIYAASSLQSGVPSRFSGSGSGTDFTLTISSLQPEDFATYYCQQSNSFPFTLGPGTKVDIK

SEQ ID NO: 24 - protein, 107 aa, Artificial Sequence
  PAIR 6 VL
DIQMTQSPSSVSASVGDRVTITCRASQGISSWLAWYQQKPGKAPQLLIYAASRLQSGVPSRFWGSGSGTDFTLTISSLQPEDFATYYCQQANNFPFTFGPGTKVDIK

SEQ ID NO: 25 - protein, 107 aa, Artificial Sequence
  PAIR 7 VL
DIQMTQSPSSVSASVGDRVTITCRASQGIFSWLAWYQQKPGKAPKLLIYAASSLQSGVPSRFSGSGSGTDFTLTISSLQPEDFAIYYCQQANSVPITFGQGTRLEIK

SEQ ID NO: 26 - protein, 109 aa, Artificial Sequence
  PAIR 8 VL
QSVLTQPPSASGTPGQRVTISCTGSSSNIGAVYDVHWYQQLPGTAPKLLIYRNNQRPSGVPDRFSGSKSGTSASLAISGLRSEDEADYYCQTYDSSRWVFGGGTKLTVL

SEQ ID NO: 27 - protein, 110 aa, Artificial Sequence
  PAIR 9 VL
QSVLTQPPSASGTPGQRVTISCSGSSSNIGNNAVSWYQQLPGTAPKLLIYASNMRVIGVPDRFSGSKSGTSASLAISGLRSEDEADYYCGAWDDSQKALVFGGGTKLTVL

SEQ ID NO: 28 - protein, 110 aa, Artificial Sequence
  PAIR 10 VL
QSVLTQPPSASGTPGQRVTISCSGSSSNIGRNAVNWYQQLPGTAPKLLIYASNMRVSGVPDRFSGSKSGTSASLAISGLRSEDEADYYCWAWDDSQKVGVFGGGTKLTVL

SEQ ID NO: 29 - protein, 110 aa, Artificial Sequence
  PAIR 11 VL
QSVLTQPPSASGTPGQRVTISCSGSSSNIGRNAVNWYQQLPGTAPKLLIYASNMRRSGVPDRFSGSKSGTSASLAISGLRSEDEADYYCSAWDDSQKVVVFGGGTKLTVL

SEQ ID NO: 30 - protein, 110 aa, Artificial Sequence
  PAIR 12 VL
QSVLTQPPSASGTPGQRVTISCSGSSSNIGNNAVNWYQQLPGTAPKLLIYASNMRRPGVPDRFSGSKSGTSASLAISGLRSEDEADYYCEAWDDSQKAVVFGGGTKLTVL

SEQ ID NO: 31 - protein, 125 aa, Artificial Sequence
  PAIR 13 VL
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR

SEQ ID NO: 32 - protein, 108 aa, Artificial Sequence
  PAIR 14 VL
EIVLTQSPGTLSLSPGERATLSCRASQSVGINLSWYQQKPGQAPRLLIYGASHRATGIPDRFSGSGSGTDFTLTISRLEPEDFAVYYCHQYSQSPPFTFGGGTKVEIK

SEQ ID NO: 33 - protein, 108 aa, Artificial Sequence
  PAIR 15 VL
EIVLTQSPGTLSLSPGERATLSCRASQSVGINLSWYQQKPGQAPRLLIYGASHRLTGIPDRFSGSGSGTDFTLTISRLEPEDFAVYYCHQYSQPPPFTFGGGTKVEIK

SEQ ID NO: 34 - protein, 108 aa, Artificial Sequence; identical to SEQ ID NO: 33
  PAIR 16 VL

SEQ ID NO: 35 - protein, 106 aa, Artificial Sequence
  PAIR 17 VL
DIQMTQSPSSLSASVGDRVTITCKASQNINKHLDWYQQKPGKAPKLLIYFTNNLQTGVPSRFSGSGSGTDFTLTISSLQPEDFATYYCFQYNQGWTFGGGTKVEIK

SEQ ID NO: 36 - protein, 111 aa, Artificial Sequence
  PAIR 18 VL
EIVLTQSPATLSLSPGERATLSCRASESVAKYGLSLLNWFQQKPGQPPRLLIFAASNRGSGIPARFSGSGSGTDFTLTISSLEPEDFAVYYCQQSKEVPFTFGQGTKVEIK

SEQ ID NO: 37 - protein, 5 aa, Artificial Sequence
  PAIR 1 HCDR1
SYAMS

SEQ ID NO: 38 - protein, 17 aa, Artificial Sequence
  PAIR 1 HCDR2
GISAIDQSTYYADSVKG

SEQ ID NO: 39 - protein, 17 aa, Artificial Sequence
  PAIR 1 HCDR3
QKFMQLWGGGLRYPFGY

SEQ ID NO: 40 - protein, 11 aa, Artificial Sequence
  PAIR 1 LCDR1
SGEGMGDKYAA

SEQ ID NO: 41 - protein, 7 aa, Artificial Sequence
  PAIR 1 LCDR2
RDTKRPS

SEQ ID NO: 42 - protein, 9 aa, Artificial Sequence
  PAIR 1 LCDR3
GVIQDNTGV

SEQ ID NO: 43 - protein, 34 aa, artificial sequence
  N terminal His10/Avitag/Factor Xa protease cleavage site
MHHHHHHHHHHAAGLNDIFEAQKIEWHEAAIEGR

SEQ ID NO: 44 - protein, 159 aa, artificial sequence
  IL-33-01
SITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 45 - protein, 159 aa, artificial sequence
  IL-33-16
SITGISPITEYLASLSTYNDQSITFALEDESYEIYVEDLKKDEKKDKVLLSYYESQHPSNESGDGVDGKMLMVTLSPTKDFWLHANNKEHSVELHKSEKPLPDQAFFVLHNMHSNSVSFESKTDPGVFIGVKDNHLALIKVDSSENLSTENILFKLSET

SEQ ID NO: 46 - protein, 15 aa, artificial sequence
  Avitag sequence motif
GLNDIFEAQKIEWHE

SEQ ID NO: 47 - DNA, 19 bp, artificial sequence
  gRNA vector targeting RAGE exon 3
tgaggggattttccggtgc

SEQ ID NO: 48 - DNA, 19 bp, artificial sequence
  RAGE forward primer
gttgcagcctcccaacttc

SEQ ID NO: 49 - DNA, 20 bp, artificial sequence
  RAGE reverse primer
aatgaggccagtggaagtca